(19) United States
(12) Patent Application Publication
(10) Pub. No.: US 2003/0050259 A1
(43) Pub. Date: Mar. 13, 2003

(54) Method and reagent for the treatment of cardiac disease

(76) Inventors: Lawrence Blatt, Boulder, CO (US); James A. McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/730,289

(22) Filed: Dec. 5, 2000

Related U.S. Application Data
(60) Provisional application No. 60/169,100, filed on Dec. 6, 1999.

(51) Int. Cl. A61K 48/00; C07H 21/02

(52) U.S. Cl. 514/44; 536/23.1

(57) ABSTRACT

Nucleic acid molecules, including antisense and enzymatic nucleic acid molecules, such as hammerhead ribozymes, DNAzymes, and antisense, which modulate the expression of molecular targets impacting the development and progression of heart disease and failure, in particular, targeting the expression of phospholamban gene are described.

### BACKGROUND OF THE INVENTION

The present invention concerns compounds, compositions, and methods for the study, diagnosis, and treatment of degenerative and disease states related to cardiac dysfunction.

The following is a brief description of the current understanding in the biology of cardiac disease. The discussion is not meant to be complete and is provided only for understanding the invention that follows. The summary is not an admission that any of the work described below is prior art to the claimed invention.

Cardiac disease leading to heart failure is the leading cause of combined morbidity and mortality in the developed world. Nearly twenty million people worldwide suffer from heart failure related disease. An estimated five million Americans are afflicted with congestive heart failure (CHF), with 400,000 new cases diagnosed each year. In the US, cardiac disease associated failure results in approximately 40,000 deaths per year, and is associated with an additional 250,000 deaths (Harnish, 1999, Drug & Market Development, 10, 114-119). Heart failure related disease represents a major public health issue due to an overall increase in prevalence and incidence in aging populations with a greater proportion of survivors of acute myocardial infarction (AMI) (Kannel et al., 1994, Br. Heart. J., 72 (suppl), 3). Heart failure related disease represents the most common reason for hospitalization of elderly patients in the US. The resulting life expectancy of these patients is less than that of many common cancers, with five year survival rates for men and women at only 25% and 38% respectively, and with one year mortality rates for severe heart failure at 50% (Ho et al., 1993, Circulation, 88, 107).

Heart disease is characterized by a progressive decrease in cardiac output resulting from insufficient pumping activity of the diseased heart. The resulting venous back-pressure results in peripheral and pulmonary dysfunctional congestion. The heart responds to a variety of mechanical, hemodynamic, hormonal, and pathological stimuli by increasing muscle mass in response to an increased demand for cardiac output. The resulting transformation of heart tissue (myocardial hypertrophy) can arise as a result of genetic, physiologic, and environmental factors, and represents an early indication of clinical heart disease and an important risk factor for subsequent heart failure (Hunter and Chien, 1999, New England J. of Medicine, 99, 313-322).

Coronary heart disease is a predominant factor in the development of the cardiac disease state, along with prior AMI, hypertension, diabetes mellitus, and valvular heart disease. Diagnosis of cardiac disease includes determination of coronary heart disease associated left ventricular systolic dysfunction (LVSD) and/or left ventricular diastolic dysfunction (LVDD) by echocaardiographic imaging (Cleland, 1997, Dis Management Health Outcomes, 1, 169). Promising diagnosis may also rely on assaying atrial natriuretic peptide (ANP) and brain natriuretic peptide (BNP) concentrations. ANP and BNP levels are indicative of the level of ventricular dysfunction (Davidson et al., 1996, Am. J. Cardiol., 77, 828).

Current treatment strategies for cardiac disease associated failure are varied. Diuretics are often used to reduce pulmonary edema and dyspnea in patients with fluid overload, and are usually used in conjunction with angiotensin converting enzyme (ACE) inhibitors for vasodilation. Digoxin is another popular choice for treating cardiac disease as an ionotropic agent, however, doubts remain concerning the long-term efficacy and safety of Digoxin (Harnish, 1999, Drug & Market Development, 10, 114-119). Carvedilol, a beta-blocker, has been introduced to complement the above treatments in order to slow down the progression of cardiac disease. Antiarrhythmic agents can be used in order to reduce the risk of sudden death in patients suffering from cardiac disease. Lastly, heart transplants have been effective in the treatment of patients with advanced stages of cardiac disease, however, the limited supply of donor hearts greatly limits the scope of this treatment to the broad population (Harnish, 1999, Drug & Market Development, 10, 114-119).

Whereby the above treatment strategies can all improve morbidity and mortality associated with cardiac disease, the only existing definitive approach to curing the diseased heart is replacement by transplant. Even a healthy, transplanted heart can become diseased in response to the various stresses of mechanical, hemodynamic, hormonal, and pathological stimuli associated with extrinsic risk factors. As such there exists the need for therapeutics effective in reversing the physiological changes associated with cardiac disease.

Myocardial hypertrophy and apoptosis are the underlying degenerative process associated with cardiac hypertrophy and failure. A variety of signaling pathways are involved in the progression of myocardial hypertrophy and myocardial apoptosis. Genetic studies have been instrumental in elucidating these pathways and their involvement in cardiac disease through in vitro assays of cardiac muscle cells and in vivo studies of genetically engineered animals.

Studies in which the expression of specific genes have been altered in cardiac myocytes have shown that specific peptide hormones, growth factors, and cytokines can activate various features of the hypertrophic response (Hunter and Chien, 1999, New England J of Medicine, 99, 313-322). Particular substances that have been characterized from these studies include potential therapeutic and molecular targets involved in heart failure. Hunter et al., in Chien, KR, ed. Molecular basis of heart disease: a companion to Braunwald's Heart Disease, Philadelphia: W. B. Saunders, 1999:211-250, describe classes of therapeutic and molecular targets involved in heart failure including:

Endothelin 1 and angiotensin II receptor antagonists, and antagonists of ras, p38, and c-jun N-terminal kinase (JNK) for inhibition of pathologic hypertrophy.

Insulin like growth factor I and growth hormone receptor stimulation for promotion of physiologic hypertrophy.

β-adrenergic receptor blockers for inhibition of neurohumoral over stimulation.

Phospholamban and Sarcolipin small molecule inhibitors for relief of sarcoplasmic reticulum calcium ATPase inhibition to provide enhancement of myocardial contractile and relaxation responses.

Small molecule inhibitors of β-adrenergic receptor kinase to counteract the desensitization of G protein coupled receptor kinases in order to provide enhancement of myocardial contractile and relaxation responses.

Enhancement of angiogenic growth factors (VEGF, FGF-5) for relief of energy deprivation in cardiac tissues.

Promoters of myocyte survival including gp 130 ligands (cardiotrophin 1), and Neuregulin for the inhibition of apoptosis of myocytes.

Inhibitors of apoptosis such as Caspase inhibitors for the inhibition of apoptosis of myocytes.

Inhibitors of cytokines such as TNFα for the inhibition of apoptosis of myocytes.

Congestive heart failure, heart failure, dilated cardiomyopathy and pressure overload hypertrophy are nonlimiting examples of disorders and disease states that can be associated with the above classes of molecular targets.

The failure of cardiac contractile performance leading to cardiac disorders and disease, governed by impairment of cardiac excitationcontraction coupling, points to the importance of the signaling pathways involved in this process. The release and uptake of cytosolic Ca²⁺ by the sarcoplasmic reticulum plays an integral role in each cycle of cardiac contraction and excitation (Minamisawa et al., 1999, Cell, 99, 313-322). The process of Ca²⁺ reuptake is mediated by the cardiac sarcoplasmic reticulum Ca²⁺ ATPase (SERCA2a). SERCA2a activity is regulated by phospholamban, a p52 muscle specific sarcoplasmic reticulum phosphoprotein (Koss et al., 1996, Circ. Res., 79, 1059-1063, and Simmerman et al., 1998, Physiol. Rev., 78, 921-947). In its active, unphosphorylated state, phospholamban is a potent inhibitor of SERCA2a activity. Phosphorylation of phospholamban at serine 16 by cyclic AMP-dependent protein kinase (PKA) or calmodulin kinase, results in the inhibition of phospholamban interaction with SERCA2a. This phosphorylation event is predominantly responsible for the proportional increase in the rate of Ca²⁺ uptake into the sarcoplasmic reticulum and resultant ventricular relaxation (Tada et al., 1982, Mol. Cell. Biochem., 46, 73-95, and Luo et al., 1998, J. Biol. Chem., 273, 4734-4739).

For example, Pystynen et al., International PCT publication No. WO 99/00132, describe bisethers of 1-oxa, aza and thianaphthalen-2-ones as small molecule inhibitors of phospholamban for increasing coronary flow via direct dilation of the coronary arteries.

Pystynen et al., International PCT publication No. WO 99/15523, describe bisethers of 1-oxa, aza and thianaphthalen-2-ones as small molecule inhibitors of phospholamban that are useful for treating heart failure.

The efficacy of the above mentioned treatment strategies is limited. Small molecule inhibition of a molecular target is often limited by toxicity, which can restrict dosing and overall efficacy.

He et al., 1999, Circulation, 100, 974-980, describe endogenous expression of mutant phospholamban and phospholamban antisense RNA to investigate the corresponding effect on SERCA2a activity and cardiac myocyte contractility.

### SUMMARY OF THE INVENTION

Since, as indicated in the Background, a proportional decrease in Ca²⁺ uptake is a hallmark feature of heart failure (Sordahl et al., 1973, Am. J. Physiol., 224, 497-502) and since an increase in the relative ratio of phospholamban to SERCA2a is an important determinant of sarcoplasmic reticulum dysfunction in heart failure (Hasenfuss, 1998, Cardiovasc. Res., 37, 279-289), the targeting of phospholamban and related regulatory factors as therapeutic targets for heart disorders should prove valuable for cardiac indications.

A more attractive approach to the treatment of heart disease then those described above, involve the use of ribozymes and/or antisense constructs to modulate the expression of target molecules involved in heart failure. The use of nucleic acid molecules of the instant invention permits highly specific regulation of the molecular targets of interest.

Thus, the invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups (Cook et al., U.S. Pat. No. 5,359,051) and methods for their use to modulate the expression of molecular targets impacting the development and progression of heart disorders, disease and failure.

In a preferred embodiment, the invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups (Cook et al., U.S. Pat. No. 5,359,051)] and methods for their use to modulate the expression of phospholamban (PLN) (accession NM_{—}002667), sarcolipin (SLN) (accession NM_{—}003063), angiotensin II receptor (accession U20860), endothelin 1 receptor (accession NM_{—}001957), K-ras (accession NM_{—}004985), p38 (accession AF092535), c-jun N-terminal kinase (accession NM_{—}002750, L31951, NM_{—}002753), growth hormone receptor (accession NM_{—}000163), insulin-like growth factor I receptor (accession NM_{—}000875), β1-adrenergic receptor (accession NM₁₃ 000024), β1-adrenergic receptor kinase (accession NM_{—}001619, NM_{—}005160), VEGF receptor (accession U43368, M27281 X15997), fibroblast growth factor 5 (accession NM_{—}004464), cardiotrophin I (accession NM_{—}001330), neuregulin (accession AF009227), TNF-alpha (accession X02910 X02159), PI3 kinase (accession NM_{—}006218, NM_{—}006219, U86453, NM_{—}002649, M61906), and AKT kinase (accession NM_{—}005163, M77198).

In a preferred embodiment, the invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups (Cook et al., U.S. Pat. No. 5,359,051)] and methods for their use to down regulate or inhibit the expression of phospholamban (PLN) Phospholamban is commonly referred to by the acronyms selected from the group: PLB, PLM and PLN. This use of PLN to signify phospholamban is used herein for simplicity in the description of the instant invention.

In a preferred embodiment, the invention features the use of one or more of the nucleic acid-based techniques independently or in combination to inhibit the expression of the genes encoding phospholamban (PLN). Specifically, the invention features the use of nucleic acid-based techniques to specifically inhibit the expression of phospholamban (PLN) gene.

In another preferred embodiment, the invention features the use of an enzymatic nucleic acid molecule, preferably in the hammerhead, NCH, G-cleaver and/or DNAzyme motif, to inhibit the expression of phospholamban gene.

By “inhibit” it is meant that the activity of phospholamban or level of RNAs or equivalent RNAs encoding one or more protein subunits of phospholamban is reduced below that observed in the absence of the nucleic acid. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of for example, an oligonucleotide with scrambled sequence or with mismatches. In another embodiment, inhibition of phospholamban genes with the nucleic acid molecule of the instant invention is greater than in the presence of the nucleic acid molecule than in its absence.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “enzymatic portion” or “catalytic domain” is meant that portionregion of the enzymatic nucleic acid molecule essential for cleavage of a nucleic acid substrate (for example see FIG. 1).

By “substrate binding arm” or “substrate binding domain” is meant that portionregion of a ribozyme which is complementary to (i.e., able to base-pair with) a portion of its substrate. Generally, such complementarity is 100%, but can be less if desired. For example, as few as 10 bases out of 14 may be base-paired. Such arms are shown generally in FIG. 1. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides and of sufficient length to stably interact with the target RNA; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By DNAzyme is meant, an enzymatic nucleic acid molecule lacking a 2′-OH group. In particular embodiments the enzymatic nucleic acid molecule may have an attached linker(s) or other attached or associated groups, moieties, or chains containing one or more nucleotides with 2′-OH groups.

By “sufficient length” is meant an oligonucleotide of greater than or equal to 3 nucleotides.

By “stably interact“ is meant, interaction of the oligonucleotides with target nucleic acid (e.g., by forming hydrogen bonds with complementary nucleotides in the target under physiological conditions).

By “equivalent” RNA to phospholamban is meant to include those naturally occurring RNA molecules having homology (partial or complete) to phospholamban proteins or encoding for proteins with similar function as phospholamban in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homolog” is meant the nucleotide sequence of two or more nucleic acid molecules is partially or completely identical.

By “antisense nucleic acid” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review see Stein and Cheng, 1993 Science 261, 1004). Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “2-5A antisense chimera” it is meant, an antisense oligonucleotide containing a 5′ phosphorylated 2′-5′-linked adenylate residues. These chimeras bind to target RNA in a sequence-specific manner and activate a cellular 2-5A-dependent ribonuclease which, in turn, cleaves the target RNA (Torrence et al., 1993 Proc. Nati. Acad. Sci. USA 90, 1300).

By “triplex DNA” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504).

By “gene” it is meant a nucleic acid that encodes an RNA.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785. A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

At least seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in phospholamban-specific RNAs represent a novel therapeutic approach to treat a variety of pathologic indications, including, pressure overload hypertrophy, dilated cardiomyopathy, congestive heart failure, and sudden death.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., U.S. Ser. No. 09/301,511) and Zinzyme (Beigelman et al., U.S. Ser. No. 09/301,511) can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has a lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding phospholamban proteins (specifically phospholamban gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

By “highly conserved sequence region” is meant a nucleotide sequence of one or more regions in a target gene does not vary significantly from one generation to the other or from one biological system to the other.

The nucleic acid-based inhibitors of phospholamban expression are useful for the prevention of the diseases and conditions, for example, heart failure, congestive heart failure, pressure overload hypertrophy, dilated cardiomyopathy, and any other diseases or conditions that are related to the levels of phospholamban in a cell or tissue.

By “related” is meant that the reduction of phospholamban expression (specifically phospholamban gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to IX. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “consists essentially of” is meant that the active ribozyme contains an enzymatic center or core equivalent to those in the examples, and binding arms able to bind mRNA such that cleavage at the target site occurs. Other sequences may be present which do not interfere with such cleavage. Thus, a core region may, for example, include one or more loop or stem-loop structures, which do not prevent enzymatic activity. “X” in the sequences in Tables III and IV can be such a loop. A core sequence for a hammerhead ribozyme can be CUGAUGAG X CGAA where X=GCCGUUAGGC or other stem II region known in the art.

In another aspect of the invention, ribozymes or antisense molecules that cleave target RNA molecules and inhibit phospholamban (specifically phospholamban gene) activity are expressed from transcription units inserted into DNA or RNA vectors. The recombinant vectors are preferably DNA plasmids or viral vectors. Ribozyme or antisense expressing viral vectors could be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors might be repeatedly administered as necessary. Once expressed, the ribozymes or antisense bind to the target RNA and inhibit its function or expression. Delivery of ribozyme or antisense expressing vectors could be systemic, such as by intravenous or intramuscular administration, by administration to target cells ex-planted from the patient followed by reintroduction into the patient, or by any other means that would allow for introduction into the desired target cell.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

The nucleic acid molecules of the instant invention, individually, or in combination or in conjunction with other drugs, can be used to treat diseases or conditions discussed above. For example, to treat a disease or condition associated with the levels of phospholamban, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat heart disease and heart failure.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., phospholamban) capable of progression and/or maintenance of heart disorders, disease and failure.

In another preferred embodiment, the invention features nucleic acid-based techniques (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of phospholamban gene expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure models for seven different classes of enzymatic nucleic acid molecules. Arrow indicates the site of cleavage.—indicate the target sequence. Lines interspersed with dots are meant to indicate tertiary interactions.—is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme:: I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is>1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧ is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

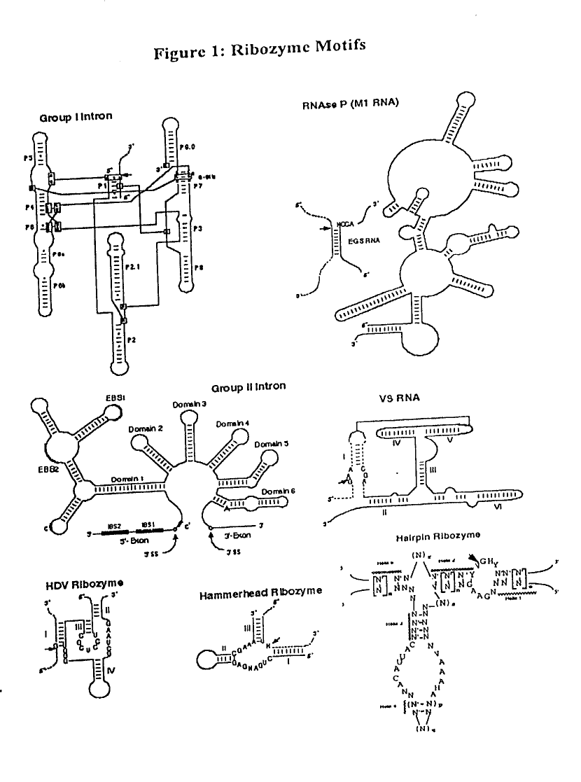

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

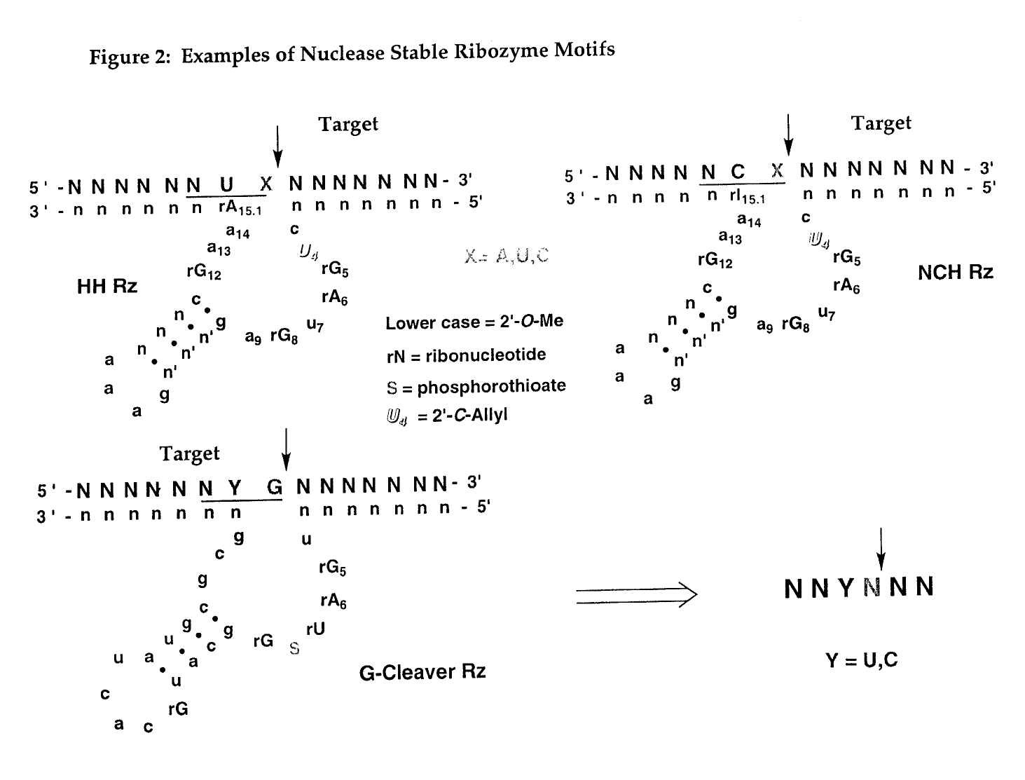

FIG. 3 shows an example of the Amberzyme ribozyme motif that is chemically stabilized (see for example Beigelman et al., U.S. Ser. No. 09/301,511; also referred to as Class I Motif).

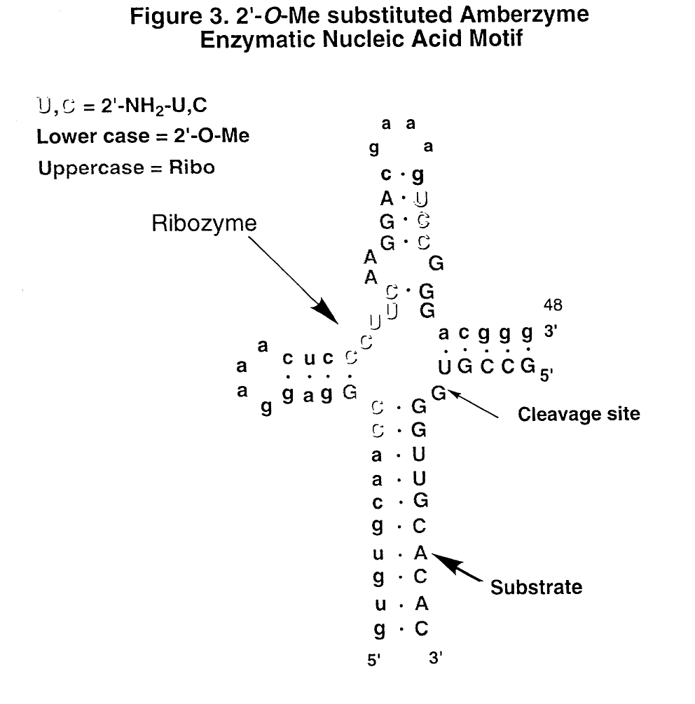

FIG. 4 shows an example of the Zinzyme A ribozyme motif that is chemically stabilized (see for example Beigelman et al., U.S. Ser. No. 09/301,511; also referred to as Class A Motif).

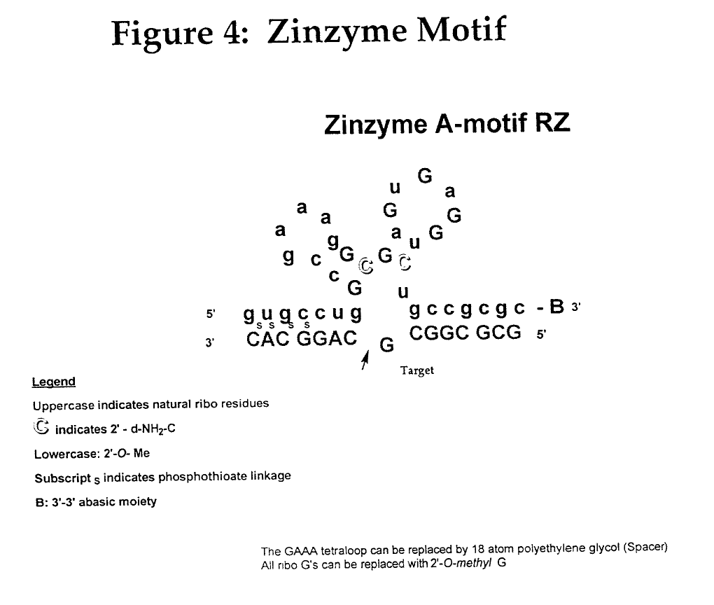

FIG. 5 shows an example of a DNAzyme motif described by Santoro et al., 1997, PNAS, 94, 4262.

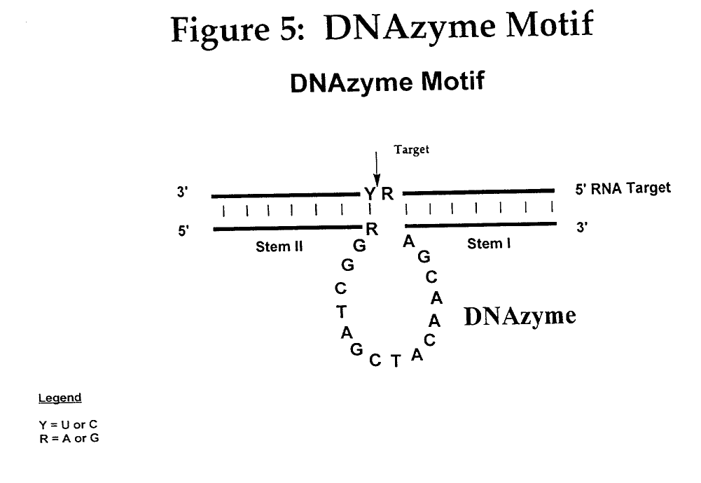

### MECHANISM OF ACTION OF NUCLEIC ACID MOLECULES OF THE INVENTION

Antisense: Antisense molecules may be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone-modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. Ser. No. 60/082,404 which was filed on Apr. 20, 1998; Hartmann et al., U.S. Ser. No. 60/101,174 which was filed on Sep. 21, 1998) all of these are incorporated by reference herein in their entirety.

Triplex Forming Oligonucleotides (TFO): Single-stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth, supra)

2-5A Antisense Chimera: The 2-5A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures may be covalently linked to antisense molecules to form chimeric oligonucleotides capable of RNA cleavage (Torrence, supra). These molecules putatively bind and activate a 2-5A dependent RNase, the oligonucleotideenzyme complex then binds to a target RNA molecule which can then be cleaved by the RNase enzyme.

Enzymatic Nucleic Acid: Seven basic varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent phospholamban protein expression and can be used to treat disease or diagnose disease associated with the levels of phospholamban.

The enzymatic nature of a ribozyme has significant advantages, such as the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieved efficient cleavage in vitro (Zaug et al., 324, Nature 429 1986 ; Uhlenbeck, 1987 Nature 328, 596; Kim et al., 84 Proc. Nat. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250.

### Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Ribozymes and antisense to such targets are designed as described in those applications and synthesized to be tested in vitro and in vivo, as also described. The sequence of human phospholamban RNAs were screened for optimal enzymatic nucleic acid and antisense target sites using a computer folding algorithm. Antisense, hammerhead, DNAzyme, NCH, or G-Cleaver ribozyme bindingcleavage sites were identified. These sites are shown in Tables III to IX (all sequences are 5′ to 3′ in the tables; X can be any base-paired sequence, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, or G-Cleaver ribozyme binding/cleavage sites were identified. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core were eliminated from consideration. Varying binding arm lengths can be chosen to optimize activity.

Antisense, hammerhead, DNAzyme, NCH, or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

### Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the hairpin ribozymes) are preferably used for exogenous delivery. The simple structure of these molecules increases the ability of the nucleic acid to invade targeted regions of RNA structure. Exemplary molecules of the instant invention were chemically synthesized, and others can similarly be synthesized. Oligodeoxyribonucleotides were synthesized using standard protocols as described in Caruthers et al., 1992, Methods in Enzymology 211, 3-19, and is incorporated herein by reference.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses were conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, were 97.5-99%. Other oligonucleotide synthesis reagents for the 394 Applied Biosystems, Inc. synthesizer; detritylation solution was 3% TCA in methylene chloride (ABI); capping was performed with 16% N-methyl imidazole in THF (ABI) and 10% acetic anhydride/10% 2,6-lutidine in THF (ABI); oxidation solution was 16.9 mM I₂, 49 mM pyridine, 9% water in THF (PERSEPTIVE™). Burdick & Jackson Synthesis Grade acetonitrile was used directly from the reagent bottle. S-Ethyltetrazole solution (0.25 M in acetonitrile) was made up from the solid obtained from American International Chemical, Inc.

Deprotection of the RNA was performed using either a two-pot or one-pot protocol. For the two-pot protocol, the polymer-bound trityl-on oligoribonucleotide was transferred to a 4 mL glass screw top vial and suspended in a solution of 40% aq. methylamine (1 mL) at 65° C. for 10 min. After cooling to −20° C., the supernatant was removed from the polymer support. The support was washed three times with 1.0 mL of EtOH:MeCN:H2O/3:1:1, vortexed and the supernatant was then added to the first supernatant. The combined supernatants, containing the oligoribonucleotide, were dried to a white powder. The base deprotected oligoribonucleotide was resuspended in anhydrous TEA/HF/NMP solution (300 μL of a solution of 1.5 mL N-methylpyrrolidinone, 750 μL TEA and 1 mL TEA●3HF to provide a 1.4 M HF concentration) and heated to 65° C. After 1.5 h, the oligomer was quenched with 1.5 M NH₄HCO₃.

Alternatively, for the one-pot protocol, the polymer-bound trityl-on oligoribonucleotide was transferred to a 4 mL glass screw top vial and suspended in a solution of 33% ethanolic methylamine/DMSO: 1/1 (0.8 mL) at 65° C. for 15 min. The vial was brought to r.t. TEA●3HF (0.1 mL) was added and the vial was heated at 65° C. for 15 min. The sample was cooled at −20° C. and then quenched with 1.5 M NH₄HCO₃.

For purification of the trityl-on oligomers, the quenched NH₄HCO₃ solution was loaded onto a C-18 containing cartridge that had been prewashed with acetonitrile followed by 50 mM TEAA. After washing the loaded cartridge with water, the RNA was detritylated with 0.5% TFA for 13 min. The cartridge was then washed again with water, salt exchanged with 1 M NaCl and washed with water again. The oligonucleotide was then eluted with 30% acetonitrile.

Inactive hammerhead ribozymes or binding attenuated control (BAC) oligonucleotides) were synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields were >98% (Wincott et al., 1995 Nucleic Acids Res. 23, 2677-2684). Those of ordinary skill in the art will recognize that the scale of synthesis can be adapted to be larger or smaller than the example described above, including, but not limited to 96-well format all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the ribozymes and antisense constructs that are chemically synthesized, useful in this study, are shown in Tables III to IX. Those in the art will recognize that these sequences are representative only of many more such sequences where the enzymatic portion of the ribozyme (all but the binding arms) is altered to affect activity. The ribozyme and antisense construct sequences listed in Tables III to IX may be formed of ribonucleotides or other nucleotides or non-nucleotides. Such ribozymes with enzymatic activity are equivalent to the ribozymes described specifically in the Tables.

### Optimizing Activity of the Nucleic Acid Molecule of the Invention.

Chemically synthesizing nucleic acid molecules with modifications (base, sugar and/or phosphate) that prevent their degradation by serum ribonucleases may increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All of these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; all of these references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore, when designing nucleic acid molecules, the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules (e.g., enzymatic nucleic acid molecules and antisense nucleic acid molecules) delivered exogenously should optimally be stable within cells until translation of the target RNA has been inhibited long enough to reduce the levels of the undesirable protein. This period of time varies between hours to days depending upon the disease state. Clearly, exogenously delivered nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of these the nucleic acid-based molecules of the invention will lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules may also include combinations of different types of nucleic acid molecules.

By “enhanced enzymatic activity” is meant to include activity measured in cells and/or in vivo where the activity is a reflection of both catalytic activity and ribozyme stability. In this invention, the product of these properties is increased or not significantly (less that 10 fold) decreased in vivo compared to an all RNA ribozyme or all DNA enzyme.

In yet another preferred embodiment, nucleic acid catalysts having chemical modifications which maintain or enhance enzymatic activity is provided. Such nucleic acid is also generally more resistant to nucleases than unmodified nucleic acid. Thus, in a cell and/or in vivo the activity may not be significantly lowered. As exemplified herein, such ribozymes are useful in a cell and/or in vivo even if activity over all is reduced 10-fold (Burgin et al., 1996, Biochemistry, 35, 14090). Such ribozymes herein are said to “maintain” the enzymatic activity of an all RNA ribozyme.

In another aspect the nucleic acid molecules comprise a 5′ and/or a 3′-cap structure.

By “cap structure” is meant chemical modifications, which have been incorporated at the terminus of the oligonucleotide (see for example Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both termini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see, Beigelman et al., International PCT publication No. WO 97/26270, incorporated by reference herein). In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties (for more details, see Beaucage and Iyer, 1993, Tetrahedron 49, 1925; incorporated by reference herein). By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art. These have recently been summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

By “abasic” is meant sugar moieties lacking a base or having other chemical groups in place of a base at the 1′ position.

By “ribonucleotide” is meant a nucleotide with one of the bases adenine, cytosine, guanine, or uracil joined to the 1′-carbon of β-D-ribo-furanose.

By “unmodified nucleoside” is meant one of the bases adenine, cytosine, guanine, uracil joined to the 1′-carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which may be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

### Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules may be directly delivered ex vivo to cells or tissues with or without the aforementioned vehicles. Alternatively, the nucleic acidvehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra and Draper et al., PCT WO93/23569 which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages, is also useful. This approach may provide enhanced delivery of the drug to target cells by taking advantage of the specificity of macrophage and lymphocyte immune recognition of abnormal cells, such as cancer cells.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of the references are hereby incorporated in their totality by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect, the invention features an expression vector comprising nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features, an expression vector comprising: a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said nucleic acid sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the nucleic acid sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res. . . , 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said nucleic acid sequence is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a gene encoding at least one said nucleic acid molecule, wherein said nucleic acid sequence is operably linked to the 3′-end of said open reading frame; and wherein said nucleic acid sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a nucleic acid sequence encoding at least one said nucleic acid molecule; and wherein said nucleic acid sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said nucleic acid sequence is operably linked to the 3′-end of said open reading frame; and wherein said nucleic acid sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, or G-Cleaver ribozyme molecules and bindingcleavage sites within phospholamban RNA.

### Example 1

### Identification of Potential Target Sites in Human Phospholamban RNA

The sequence of human phospholamban was screened for accessible sites using a computer folding algorithm. Regions of the RNA that did not form secondary folding structures and contained potential ribozyme and/or antisense binding/cleavage sites were identified. The sequences of these cleavage sites are shown in tables III-IX.

### Example 2

### Selection of Enzymatic Nucleic Acid Cleavage Sites in Human phospholamban RNA

Ribozyme target sites were chosen by analyzing sequences of Human phospholamban (Genbank sequence accession number: NM_{—}003219) and prioritizing the sites on the basis of folding. Ribozymes were designed that could bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or Blocking of Phospholamban RNA

Ribozymes and antisense constructs were designed to anneal to various sites in the RNA message. The binding arms of the ribozymes are complementary to the target site sequences described above, while the antisense constructs are fully complimentary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were >98%.

Ribozymes and antisense constructs were also synthesized from DNA templates using bacteriophage T7 RNA polymerase (Milligan and Uhlenbeck, 1989, Methods Enzymol. 180, 51). Ribozymes and antisense constructs were purified by gel electrophoresis using general methods or were purified by high pressure liquid chromatography (HPLC; See Wincott et al., supra; the totality of which is hereby incorporated herein by reference) and were resuspended in water. The sequences of the chemically synthesized ribozymes and antisense constructs used in this study are shown below in Table III-IX.

### Example 4

### Ribozyme Cleavage of Phospholamban RNA Target in Vitro

Ribozymes targeted to the human phospholamban RNA are designed and synthesized as described above. These ribozymes can be tested for cleavage activity in vitro, for example using the following procedure. Target sequences and in the nucleotide locations within the phospholamban RNA are given in Tables III-IX.

Cleavage Reactions: Full-length or partially full-length, internally-labeled target RNA for ribozyme cleavage assay is prepared by in vitro transcription in the presence of [a-³²p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2X concentration of purified ribozyme in ribozyme cleavage buffer (50 mM Tris-HCl, pH 7.5 at 37° C., 10 mM MgCl₂) and the cleavage reaction was initiated by adding the 2X ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

### Tissue Distribution of BrdU-labeled Antisense in Mice

CD1 mice were injected with a single bolus (30 mg/kg) of a BrdU-labeled antisense oligonucleotide or a similar molar amount of BrdU (as a control). At various time points (30 min, 2 h and 6 h), mice were sacrificed and major tissues isolated and fixed. Distribution of antisense oligonucleotides was determined by probing with an anti-BrdU antibody and immunohistochemical staining. Tissue slices were probed with an anti-BrdU antibody followed by a reporter enzyme-conjugated second antibody and finally an enzyme substrate. Visualization of the colored product by microscopy indicated nuclear staining, demonstrating effective distribution of antisense oligonucleotide in cardiac tissue.

### Example 6

### Tissue Distribution of BrdU-labeled Ribozymes in Monkey

Rhesus monkeys were dosed with BrdU-labeled ribozyme by intravenous bolus injection at 0.1, 1.0, and 10 mg/kg once daily over five days. Saline injection was used in control animals. Animals were sacrificed and major tissues isolated and fixed. Tissue samples were probed with an anti-BrdU antibody followed by a reporter enzyme-conjugated second antibody and finally an enzyme substrate. Significant quantities of chemically modified ribozyme are detected in cardiac tissue following this dosing regimen.

### Cell Culture Models

Various methods have been developed to assay phospholamban activity in vitro and in vivo. Holt et al., 1999, J. Mol. Cell. Cardiol., 31, 645-656, describe a cell culture model in which thyroid hormone control of contraction and the Ca²⁺−ATPase/phospholamban complex is studied in adult rat ventricular myocytes. Slack et al. 1997, J. Biol. Chem., 272, 18862-18868, describe studies in which the ectopic expression of phospholamban in mouse fast-twitch skeletal muscle cells alters sarcoplasmic reticulum Ca²⁺ transport and muscle relaxation. MacLennan et al., 1996, Soc. Gen. Physiol. Ser., 51, 89-103, in a review of regulatory interactions between calcium ATPases and phospholamban describe phospholamban/Ca²⁺−ATPase interactions in protein expressed in heterologous cell culture experiments. Cornwell et al., 1991, Mol. Pharmacol., 40,923-931, describe the regulation of sarcoplasmic reticulum protein phosphorylation by localized cyclic GMP-dependent protein kinase in vascular smooth muscle cells.

### Animal Models

Minamisawa et al., 1999, Cell, 99, 313-322, describe a phospholamban knockout mouse model which affords protection from induced dilated cardiomyopathy. Dillmann et al., 1999, Am. J. Cardiol., 83, 89H-91H, describe a transgenic rat model for the study of altered expression of calcium regulatory proteins, including phospholamban, and their effect on myocyte contractile response. LekanneDeprez et al., 1998, J. Mol. Cell. Cardiol., 30, 1877-1888, describe a rat pressure-overload model to investigate alterations in gene expression of phospholamban, atrial natriuretic peptide (ANP), sarcoplasmic endoplasmic reticular calcium ATPase 2 (SERCA2), collagen IIIα1, and calsequestrin (CSQ). Jones et al., 1998, J. Clin. Invest., 101, 1385-1393, describe a mouse model for investigating the regulation of calcium signaling in transgenic mouse cardiac myocytes overexpressing calsequestrin. In this study, the upregulation and downregulation of calcium uptake and release proteins were determined, including phospholamban. Lorenz et al., 1997, Am J. Physiol., 273, 6, describe a mouse model for the study of regulatory effects of phospholamban on cardiac function in intact mice. This study makes use of animal models with altered levels of phospholamban, to permit in vivo evaluation of the physiological role of phospholamban. Arai et al., 1996, Saishin lgaku, 51, 1095-1104, presents a review article of gene targeted animal models expressing cardiovascular abnormalities. The study of phospholamban and other protein expression modification effects in mice is presented. Wankerl et al., 1995, J. Mol. Med., 73, 487-496, presents a review article describing the study of calcium transport proteins in the non-failing and failing heart. Animal models investigating the major calcium handling myocardial proteins, including phospholamban, are described. These models, as well as others, may be used to evaluate the effect of treatment with nucleic acid molecules of the instant invention on cardiac function. Endpoints may be, but are not limited to, left ventricular pressure, left ventricular pressure as a function of time (LVdP/dt), and mean arterial blood pressure. Endpoints will be evaluated under basal and stimulated (cardiac load) conditions.

### Indications

Particular degenerative and disease states that can be associated with phospholamban expression modulation include, but are not limited to, congestive heart failure, heart failure, dilated cardiomyopathy and pressure overload hypertrophy:

The present body of knowledge in phospholamban research indicates the need for methods to assay phospholamban activity and for compounds that can regulate phospholamban expression for research, diagnostic, and therapeutic use.

Digoxin, Bendrofluazide, Dofetilide, and Carvedilol are non-limiting examples of pharmaceutical agents that can be combined with or used in conjunction with the nucleic acid molecules (e.g. ribozymes and antisense molecules) of the instant invention. Those skilled in the art will recognize that other drugs such as diuretic and antihypertensive compounds and therapies can be similarly be readily combined with the nucleic acid molecules of the instant invention (e.g. ribozymes and antisense molecules) and, hence, are within the scope of the instant invention.

### Diagnostic Uses

The nucleic acid molecules of this invention (e.g., ribozymes) may be used as diagnostic tools to examine genetic drift and mutations within diseased cells or to detect the presence of phospholamban RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with phospholamban-related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA will be cleaved by both ribozymes to demonstrate the relative ribozyme efficiencies in the reactions and the absence of cleavage of the “non-targeted” RNA species. The cleavage products from the synthetic substrates will also serve to generate size markers for the analysis of wild-type and mutant RNAs in the sample population. Thus, each analysis will utilize two ribozymes, two substrates and one unknown sample, which will be combined into six reactions. The presence of cleavage products can be determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., phospholamban) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

### Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns

| | |
|---|---|
| • | Size: ˜ 150 to > 1000 nucleotides. |
| • | Requires a U in the target sequence immediately 5′ of the cleavage site. |
| • | Binds 4-6 nucleotides at the 5′-side of the cleavage site. |
| • | Reaction mechanism: attack by the 3′-OH of guanosine to generate cleavage |
| | products with 3′-OH and 5′-guanosine. |
| • | Additional protein cofactors required in some cases to help folding and maintenance |
| | of the active structure. |
| • | Over 300 known members of this class. Found as an intervening sequence in |
| | Tetrahymena thermophila rRNA, fungal mitochondria, chloroplasts, phage T4, blue- |
| | green algae, and others. |
| • | Major structural features largely established through phylogenetic comparisons, |
| | mutagenesis, and biochemical studies [ⁱ, ⁱⁱ]. |
| • | Complete kinetic framework established for one ribozyme [ⁱⁱⁱ, ^{iv}, ^{v}, ^{vi}]. |
| • | Studies of ribozyme folding and substrate docking underway [^{vii}, ^{viii}, ^{ix}]. |
| • | Chemical modification investigation of important residues well established [^{x}, ^{xi}]. |
| • | The small (4-6 nt) binding site may make this ribozyme too non-specific for |
| | targeted RNA cleavage, however, the Tetrahymena group I intron has been used |
| | to repair a ”defective” -galactosidase message by the ligation of new |
| | -galactosidase sequences onto the defective message [^{xii}]. |

RNAse P RNA (M1 RNA)

| | |
|---|---|
| • | Size: ˜ 290 to 400 nucleotides. |
| • | RNA portion of a ubiquitous ribonucleoprotein enzyme. |
| • | Cleaves tRNA precursors to form mature tRNA [^{xiii}]. |
| • | Reaction mechanism: possible attack by M²⁺-OH to generate cleavage products |
| | with 3′-OH and 5′-phosphate. |
| • | RNAse P is found throughout the prokaryotes and eukaryotes. The RNA subunit has |
| | been sequenced from bacteria, yeast, rodents, and primates. |
| • | Recruitment of endogenous RNAse P for therapeutic applications is possible through |
| | hybridization of an External Guide Sequence (EGS) to the target RNA [^{xiv}, ^{xv}] |
| • | Important phosphate and 2′ OH contacts recently identified [^{xvi}, ^{xvii}] |

Group II Introns

| | |
|---|---|
| • | Size: > 1000 nucleotides. |
| • | Trans cleavage of target RNAs recently demonstrated [^{xviii}, ^{xix}]. |
| • | Sequence requirements not fully determined. |
| • | Reaction mechanism: 2′-OH of an internal adenosine generates cleavage products |
| | with 3′-OH and a “lariat” RNA containing a 3′-5′ and a 2′-5′ branch point. |
| • | Only natural ribozyme with demonstrated participation in DNA cleavage [^{xx}, ^{xxi}] in |
| | addition to RNA cleavage and ligation. |
| • | Major structural features largely established through phylogenetic comparisons [^{xxii}]. |
| • | Important 2′ OH contacts beginning to be identified [^{xxiii}] |
| • | Kinetic framework under development [^{xxiv}] |

Neurospora VS RNA

| | |
|---|---|
| • | Size: ˜ 144 nucleotides. |
| • | Trans cleavage of hairpin target RNAs recently demonstrated [^{xxv}]. |
| • | Sequence requirements not fully determined. |
| • | Reaction mechanism: attack by 2′-OH 5′ to the scissile bond to generate cleavage |
| | products with 2′,3′-cyclic phosphate and 5′-OH ends. |
| • | Binding sites and structural requirements not fully determined. |
| • | Only 1 known member of this class. Found in Neurospora VS RNA. |

Hammerhead Ribozyme
(see text for references)

| | |
|---|---|
| • | Size: ˜ 13 to 40 nucleotides. |
| • | Requires the target sequence UH immediately 5′ of the cleavage site. |
| • | Binds a variable number nucleotides on both sides of the cleavage site. |
| • | Reaction mechanism: attack by 2′-OH 5′ to the scissile bond to generate cleavage |
| | products with 2′,3′-cyclic phosphate and 5′-OH ends. |
| • | 14 known members of this class. Found in a number of plant pathogens (virusoids) |
| | that use RNA as the infectious agent. |
| • | Essential structural features largely defined, including 2 crystal structures [^{xxvi}, ^{xxvii}] |
| • | Minimal ligation activity demonstrated (for engineering through in vitro selection) |
| | [^{xxviii}] |
| • | Complete kinetic framework established for two or more ribozymes [^{xxix}]. |
| • | Chemical modification investigation of important residues well established [^{xxx}]. |

Hairpin Ribozyme

| | |
|---|---|
| • | Size: ˜ 50 nucleotides. |
| • | Requires the target sequence GUC immediately 3′ of the cleavage site. |
| • | Binds 4-6 nucleotides at the 5′-side of the cleavage site and a variable number to the |
| | 3′-side of the cleavage site. |
| • | Reaction mechanism: attack by 2′-OH 5′ to the scissile bond to generate cleavage |
| | products with 2′,3′-cyclic phosphate and 5′-OH ends. |
| • | 3 known members of this class. Found in three plant pathogen (satellite RNAs of the |
| | tobacco ringspot virus, arabis mosaic virus and chicory yellow mottle virus) which |
| | uses RNA as the infectious agent. |
| • | Essential structural features largely defined [^{xxxi}, ^{xxxii}, ^{xxxiii}, ^{xxxiv}] |
| • | Ligation activity (in addition to cleavage activity) makes ribozyme amenable to |
| | engineering through in vitro selection [^{xxxv}] |
| • | Complete kinetic framework established for one ribozyme [^{xxxvi}]. |
| • | Chemical modification investigation of important residues begun [^{xxxvii}, ^{xxxviii}]. |

Hepatitis Delta Virus (HDV) Ribozyme

| | |
|---|---|
| • | Size: ˜ 60 nucleotides. |
| • | Trans cleavage of target RNAs demonstrated [^{xxxix}]. |
| • | Binding sites and structural requirements not fully determined, although no |
| | sequences 5′ of cleavage site are required. Folded ribozyme contains a pseudoknot |
| | structure [^{xl}]. |
| • | Reaction mechanism: attack by 2′-OH 5′ to the scissile bond to generate cleavage |
| | products with 2′,3′-cyclic phosphate and 5′-OH ends. |
| • | Only 2 known members of this class. Found in human HDV. |
| • | Circular form of HDV is active and shows increased nuclease stability [^{xli}] |

ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H.. The time dependence of chemical modification reveals slow steps in the folding of a group I ribozyme. Biochemistry (1995), 34(19), 6504-12.
^{ix}Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x}Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
^{xi}Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii}Sullenger, Bruce A.; Cech, Thomas R.. Ribozyme-mediated repair of defective mRNA by targeted trans-splicing. Nature (London) (1994), 371(6498), 619-22.
^{xiii}Robertson, H. D.; Altman, S.; Smith, J. D. J. Biol. Chem., 247, 5243-5251 (1972).
^{xiv}Forster, Anthony C.; Altman, Sidney. External guide sequences for an RNA enzyme. Science (Washington, D.C., 1883-) (1990), 249(4970), 783-6.
^{xv}Yuan, Y.; Hwang, E. S.; Altman, S. Targeted cleavage of mRNA by human RNase P. Proc. Natl. Acad. Sci. U.S.A. (1992) 89, 8006-10.
^{xvi}Harris, Michael E.; Pace, Norman R.. Identification of phosphates involved in catalysis by the ribozyme RNase P RNA. RNA (1995), 1(2), 210-18.
^{xvii}Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C. T.; Collins, Richard A.. Efficient trans-cleavage of a stem-loop RNA substrate by a ribozyme derived from Neurospora VS RNA. EMBOJ. (1995), 14(2), 368-76.
^{xxvi}Scott, W. G., Finch, J. T., Aaron, K. The crystal structure of an all RNA hammerhead ribozyme:Aproposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
^{xxx}Beigelman, L., et al., Chemical modifications of hammerhead ribozymes. J. Biol. Chem., (1995) 270, 25702-25708.
^{xxxi}Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv}Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl}Perrotta, Anne T.; Been, Michael D.. A pseudoknot-like structure required for efficient self-cleavage of hepatitis delta virus RNA. Nature (London) (1991), 350(6317), 434-6.
^{xli}Puttaraju, M.; Perrotta, Anne T.; Been, Michael D.. A circular trans-acting hepatitis delta virus ribozyme. Nucleic Acids Res. (1993), 21(18), 4253-8.

TABLE II
A. 2.5 μmol Synthesis Cycle ABI 394 Instrument

| | | | Wait Time* | Wait Time* |
|---|---|---|---|---|
| Reagent | Equivalents | Amount | 2′-O-methyl | RNA |
| Phosphoramidites | 6.5 | 163 μL | 2.5 min | 7.5 |
| S-Ethyl Tetrazole | 23.8 | 238 μL | 2.5 min | 7.5 |
| Acetic Anhydride | 100 | 233 μL | 5 sec | 5 sec |
| N-Methyl Imidazole | 186 | 233 μL | 5 sec | 5 sec |
| TCA | 110.1 | 2.3 mL | 21 sec | 21 sec |
| Iodine | 11.2 | 1.7 mL | 45 sec | 45 sec |
| Acetonitrile | NA | 6.67 mL | NA | NA |

B. 0.2 μmol Synthesis Cycle ABI 394 Instrument

| | | | Wait Time* | Wait Time* |
|---|---|---|---|---|
| Reagent | Equivalents | Amount | 2′-O-methyl | RNA |
| Phosphoramidites | 15 | 31 μL | 233 sec | 465 sec |
| S-Ethyl Tetrazole | 38.7 | 31 μL | 233 min | 465 sec |
| Acetic Anhydride | 655 | 124 μL | 5 sec | 5 sec |
| N-Methyl Imidazole | 1245 | 124 μL | 5 sec | 5 sec |
| TCA | 700 | 732 μL | 10 sec | 10 sec |
| Iodine | 20.6 | 244 μL | 15 sec | 15 sec |
| Acetonitrile | NA | 2.64 mL | NA | NA |

C. 0.2 μmol Synthesis Cycle 96 well Instrument

| | | Amount | | |
|---|---|---|---|---|
| | Equivalents | 2′-O-methyl/ | Wait Time* | Wait Time* |
| Reagent | 2′-O-methyl/Ribo | Ribo | 2′-O-methyl | Ribo |
| Phosphoramidites | 33/66 | 60/120 μL | 233 sec | 465 sec |
| S-Ethyl Tetrazole | 75/150 | 60/120 μL | 233 min | 465 sec |
| Acetic Anhydride | 50/50 | 50/50 μL | 10 sec | 10 sec |
| N-Methyl Imidazole | 502/502 | 50/50 μL | 10 sec | 10 sec |
| TCA | 16,000/16,000 | 500/500 μL | 15 sec | 15 sec |
| Iodine | 6.8/6.8 | 80/80 μL | 30 sec | 30 sec |
| Acetonitrile | NA | 850/850 μL | NA | NA |

*Wait time does not include contact time during delivery.

TABLE III
Human Phospholamban (PLN) Hammerhead Ribozyme and Target Sequence

| | | | | Rz Seq |
|---|---|---|---|---|
| Pos | Substrate | Seq ID | Ribozyme | ID |
| 16 | AGAAAACU C CCCAGCUA | 1 | UAGCUGGG CUGAUGAG X CGAA AGUUUUCU | 1137 |
| 24 | CCCCAGCU A AACACCCG | 2 | CGGGUGUU CUGAUGAG X CGAA AGCUGGGG | 1138 |
| 34 | ACACCCGU A AGACUUCA | 3 | UGAAGUCU CUGAUGAG X CGAA ACGGGUGU | 1139 |
| 40 | GUAAGACU U CAUACAAC | 4 | GUUGUAUG CUGAUGAG X CGAA AGUCUUAC | 1140 |
| 41 | UAAGACUU C AUACAACA | 5 | UGUUGUAU CUGAUGAG X CGAA AAGUCUUA | 1141 |
| 44 | GACUUCAU A CAACACAA | 6 | UUGUGUUG CUGAUGAG X CGAA AUGAAGUC | 1142 |
| 54 | AACACAAU A CUCUAUAC | 7 | GUAUAGAG CUGAUGAG X CGAA AUUGUGUU | 1143 |
| 57 | ACAAUACU C UAUACUGU | 8 | ACAGUAUA CUGAUGAG X CGAA AGUAUUGU | 1144 |
| 59 | AAUACUCU A UACUGUGA | 9 | UCACAGUA CUGAUGAG X CGAA AGAGUAUU | 1145 |
| 61 | UACUCUAU A CUGUGAUG | 10 | CAUCACAG CUGAUGAG X CGAA AUAGAGUA | 1146 |
| 72 | GUGAUGAU C ACAGCUGC | 11 | GCAGCUGU CUGAUGAG X CGAA AUCAUCAC | 1147 |
| 88 | CCAAGGCU A CCUAAAAG | 12 | CUUUUAGG CUGAUGAG X CGAA AGCCUUGG | 1148 |
| 92 | GGCUACCU A AAAGAAGA | 13 | UCUUCUUU CUGAUGAG X CGAA AGGUAGCC | 1149 |
| 105 | AAGACAGU U AUCUCAUA | 14 | UAUGAGAU CUGAUGAG X CGAA ACUGUCUU | 1150 |
| 106 | AGACAGUU A UCUCAUAU | 15 | AUAUGAGA CUGAUGAG X CGAA AACUGUCU | 1151 |
| 108 | ACAGUUAU C UCAUAUUU | 16 | AAAUAUGA CUGAUGAG X CGAA AUAACUGU | 1152 |
| 110 | AGUUAUCU C AUAUUUGG | 17 | CCAAAUAU CUGAUGAG X CGAA AGAUAACU | 1153 |
| 113 | UAUCUCAU A UUUGGCUG | 18 | CAGCCAAA CUGAUGAG X CGAA AUGAGAUA | 1154 |
| 115 | UCUCAUAU U UGGCUGCC | 19 | GGCAGCCA CUGAUGAG X CGAA AUAUGAGA | 1155 |
| 116 | CUCAUAUU U GGCUGCCA | 20 | UGGCAGCC CUGAUGAG X CGAA AAUAUGAG | 1156 |
| 128 | UGCCAGCU U UUUAUCUU | 21 | AAGAUAAA CUGAUGAG X CGAA AGCUGGCA | 1157 |
| 129 | GCCAGCUU U UUAUCCUU | 22 | AAAGAUAA CUGAUGAG X CGAA AAGCUGGC | 1158 |
| 130 | CCAGCUUU U UAUCUUUC | 23 | GAAAGAUA CUGAUGAG X CGAA AAAGCUGG | 1159 |
| 131 | CAGCUUUU U AUCUUUCU | 24 | AGAAAAGU CUGAUGAG X CGAA AAAAGCUG | 1160 |
| 132 | AGCUUUUU A UCUUUCUC | 25 | GAGAAAGA CUGAUGAG X CGAA AAAAAGCU | 1161 |
| 134 | CUUUUUAU C UUUCUCUC | 26 | GAGAGAAA CUGAUGAG X CGAA AUAAAAAG | 1162 |
| 136 | UUUUAUCU U UCUCUCCA | 27 | UCGAGAGA CUGAUGAG X CGAA AGAUAAAA | 1163 |
| 137 | UUUAUCUU U CUCUCGAC | 28 | GUCGAGAG CUGAUGAG X CGAA AAGAUAAA | 1164 |
| 138 | UUAUCUUU C UCUCGACC | 29 | GGUCGAGA CUGAUGAG X CGAA AAAGAUAA | 1165 |
| 140 | AUCUUUCU C UCGACCAC | 30 | GUGGUCGA CUGAUGAG X CGAA AGAAAGAU | 1166 |
| 142 | CUUUCUCU C GACCACUU | 31 | AAGUGGUC CUGAUGAG X CGAA AGAGAAAG | 1167 |
| 150 | CGACCACC U AAAACUUC | 32 | GAAGUUUU CUGAUGAG X CGAA AGUGGUCG | 1168 |
| 151 | GACCACUU A AAACUUCA | 33 | UGAAGUUU CUGAUGAG X CGAA AAGUGGUC | 1169 |
| 157 | UUAAAACU U CAGACUUC | 34 | GAAGUCUG CUGAUGAG X CGAA AGUUUUAA | 1170 |
| 158 | UAAAACUU C AGACUUCC | 35 | GGAAGUCU CUGAUGAG X CGAA AAGUUUUA | 1171 |
| 164 | UUCAGACU U CCUGUCCU | 36 | AGGACAGG CUGAUGAG X CGAA AGUCUGAA | 1172 |
| 165 | UCAGACUU C CUGUCCUG | 37 | CAGGACAG CUGAUGAG X CGAA AAGUCUGA | 1173 |
| 170 | CUUCCUGU C CUGCUGGU | 38 | ACCAGCAG CUGAUGAG X CGAA ACAGGAAG | 1174 |
| 179 | CUGCUGGU A UCAUGGAG | 39 | CUCCAUGA CUGAUGAG X CGAA ACCAGCAG | 1175 |
| 181 | GCUGGUAU C AUGGAGAA | 40 | UUCUCCAU CUGAUGAG X CGAA AUACCAGC | 1176 |
| 193 | GAGAAAGU C CAAUACCU | 41 | AGGUAUUG CUGAUGAG X CGAA ACUUUCUC | 1177 |
| 198 | AGUCCAAU A CCUCACUC | 42 | GAGUGAGG CUGAUGAG X CGAA AUUGGACU | 1178 |
| 202 | CAAUACCU C ACUCGCUC | 43 | GAGCGAGU CUGAUGAG X CGAA AGGUAUUG | 1179 |
| 206 | ACCUCACU C GCUCAGCU | 44 | AGCUGAGC CUGAUGAG X CGAA AGUGAGGU | 1180 |
| 210 | CACUCGCU C AGCUAUAA | 45 | UUAUAGCU CUGAUGAG X CGAA AGCGAGUG | 1181 |
| 215 | GCUCAGCU A UAAGAAGA | 46 | UCUUCUUA CUGAUGAG X CGAA AGCUGAGC | 1182 |
| 217 | UCAGCUAU A AGAAGAGC | 47 | GCUCUUCU CUGAUGAG X CGAA AUAGCUGA | 1183 |
| 228 | AAGAGCCU C AACCAUUG | 48 | CAAUGGUU CUGAUGAG X CGAA AGGCUCUU | 1184 |
| 235 | UCAACCAU U GAAAUGCC | 49 | GGCAUUUC CUGAUGAG X CGAA AUGGUUGA | 1185 |
| 245 | AAAUGCCU C AACAAGCA | 50 | UGCUUGUU CUGAUGAG X CGAA AGGCAUUU | 1186 |
| 257 | AAGCACGU C AAAAGCUA | 51 | UAGCUUUU CUGAUGAG X CGAA ACGUGCUU | 1187 |
| 265 | CAAAAGCU A CAGAAUCU | 52 | AGAUUCUG CUGAUGAG X CGAA AGCUUUUG | 1188 |
| 272 | UACAGAAU C UAUUUAUC | 53 | GAUAAAUA CUGAUGAG X CGAA AUUCUGUA | 1189 |
| 274 | CAGAAUCU A UUUAUCAA | 54 | UUGAUAAA CUGAUGAG X CGAA AGAUUCUG | 1190 |
| 276 | GAAUCUAU U UAUCAAUU | 55 | AAUUGAUA CUGAUGAG X CGAA AUAGAUUC | 1191 |
| 277 | AAUCUAUU U AUCAAUUU | 56 | AAAUUGAU CUGAUGAG X CGAA AAUAGAUU | 1192 |
| 278 | AUCUAUUU A UCAAUUUC | 57 | GAAAUUGA CUGAUGAG X CGAA AAAUAGAU | 1193 |
| 280 | CUAUUUAU C AAUUUCUG | 58 | CAGAAAUU CUGAUGAG X CGAA AUAAAUAG | 1194 |
| 284 | UUAUCAAU U UCUGUCUC | 59 | GAGACAGA CUGAUGAG X CGAA AUUGAUAA | 1195 |
| 285 | UAUCAAUU U CUGUCUCA | 60 | UGAGACAG CUGAUGAG X CGAA AAUUGAUA | 1196 |
| 286 | AUCAAUUU C UGUCUCAU | 61 | AUGAGACA CUGAUGAG X CGAA AAAUUGAU | 1197 |
| 290 | AUUUCUGU C UCAUCUUA | 62 | UAAGAUGA CUGAUGAG X CGAA ACAGAAAU | 1198 |
| 292 | UUCUGUCU C AUCUUAAU | 63 | AUUAAGAU CUGAUGAG X CGAA AGACAGAA | 1199 |
| 295 | UGUCUCAU C UUAAUAUG | 64 | CAUAUUAA CUGAUGAG X CGAA AUGAGACA | 1200 |
| 297 | UCUCAUCU U AAUAUGUC | 65 | GACAUAUU CUGAUGAG X CGAA AGAUGAGA | 1201 |
| 298 | CUCAUCUU A AUAUGUCU | 66 | AGACAUAU CUGAUGAG X CGAA AAGAUGAG | 1202 |
| 301 | AUCUUAAU A UGUCUCUU | 67 | AAGAGACA CUGAUGAG X CGAA AUUAAGAU | 1203 |
| 305 | UAAUAUGU C UCUUGCUG | 68 | CAGCAAGA CUGAUGAG X CGAA ACAUAUUA | 1204 |
| 307 | AUAUGUCU C UUGCUGAU | 69 | AUCAGCAA CUGAUCAG X CGAA AGACAUAU | 1205 |
| 309 | AUGUCUCU U GCUGAUCU | 70 | AGAUCAGC CUGAUGAG X CGAA AGAGACAU | 1206 |
| 316 | UUGCUGAU C UGUAUCAU | 71 | AUGAUACA CUGAUGAG X CGAA AUCAGCAA | 1207 |
| 320 | UGAUCUGU A UCAUCGUG | 72 | CACCAUGA CUGAUGAG X CGAA ACAGAUCA | 1208 |
| 322 | AUCUGUAU C AUCGUGAU | 73 | AUCACGAU CUGAUGAG X CGAA AUACAGAU | 1209 |
| 325 | UGUAUCAU C GUGAUGCU | 74 | AGCAUCAC CUGAUGAG X CGAA AGCAUCAC | 1210 |
| 334 | GUGAUGCU U CUCUGAAG | 75 | CUUCAGAG CUGAUGAG X CGAA AGCAUCAC | 1211 |
| 335 | UGAUGCUU C UCUGAAGU | 76 | ACUUCAGA CUGAUGAG X CGAA AAGCAUCA | 1212 |
| 337 | AUGCUUCU C UGAAGUUC | 77 | GAACUUCA CUGAUGAG X CGAA AGAAGCAU | 1213 |
| 344 | UCUGAAGU U CUGCUACA | 78 | UGUAGCAG CUGAUGAG X CGAA ACUUCAGA | 1214 |
| 345 | CUGAAGUU C UGCUACAA | 79 | UUGUAGCA CUGAUGAG X CGAA AACUUCAG | 1215 |
| 350 | GUUCUGCU A CAACCUCU | 80 | AGAGGUUG CUGAUGAG X CGAA AGCAGAAC | 1216 |
| 357 | UACAACCU C UAGAUCUG | 81 | CAGAUCUA CUGAUGAG X CGAA AGGUUGUA | 1217 |
| 359 | CAACCUCU A GAUCUGCA | 82 | UGCAGAUC CUGAUGAG X CGAA AGAGGUUG | 1218 |
| 363 | CUCUAGAU C UGCAGCUU | 83 | AAGCUGCA CUGAUGAG X CGAA AUCUAGAG | 1219 |
| 371 | CUGCAGCU U GCCACAUC | 84 | GAUGUGGC CUGAUGAG X CGAA AGCUGCAG | 1220 |
| 379 | UGCCACAU C AGCUUAAA | 85 | UUUAAGCU CUGAUGAG X CGAA AUGUGGCA | 1221 |
| 384 | CAUCAGCU U AAAAUCUG | 86 | CAGAUUUU CUGAUGAG X CGAA AGCUGAUG | 1222 |
| 385 | AUCAGCUU A AAAUCUGU | 87 | ACAGAUUU CUGAUGAG X CGAA AAGCUGAU | 1223 |
| 390 | CUUAAAAU C UGUCAUCC | 88 | GGAUGACA CUGAUGAG X CGAA AUUUUAAG | 1224 |
| 394 | AAAUCUGU C AUCCCAUG | 89 | CAUGGGAU CUGAUGAG X CGAA ACAGAUUU | 1225 |
| 397 | UCUGUCAU C CCAUGCAG | 90 | CUGCAUGG CUGAUGAG X CGAA AUGACAGA | 1226 |
| 419 | AAAACAAU A UUGUAUAA | 93 | UUAUACAA CUGAUGAG X CGAA AUUGUUUU | 1227 |
| 421 | AACAAUAU U GUAUAACA | 92 | UGUUAUAC CUGAUGAG X CGAA AUAUUGUU | 1228 |
| 424 | AAUAUUGU A UAACAGAC | 93 | GUCUGUUA CUGAUGAG X CGAA ACAAUAUU | 1229 |
| 426 | UAUUGUAU A ACAGACCA | 94 | UGGUCUGU CUGAUGAG X CGAA AUACAAUA | 1230 |
| 437 | AGACCACU U CCUGAGUA | 95 | UACUCAGG CUGAUGAG X CGAA AGUGGUCU | 1231 |
| 438 | GACCACUU C CUGAGUAG | 96 | CUACUCAG CUGAUGAG X CGAA AAGUGGUC | 1232 |
| 445 | UCCUGAGU A GAAGAGUU | 97 | AACUCUUC CUGAUGAG X CGAA ACUCAGGA | 1233 |
| 453 | AGAAGAGU U UCUUUGUG | 98 | CACAAAGA CUGAUGAG X CGAA ACUCUUCU | 1234 |
| 454 | GAAGAGUU U CUUUGUGA | 99 | UCACAAAG CUGAUGAG X CGAA AACUCUUC | 1235 |
| 455 | AAGAGUUU C UUUGUGAA | 100 | UUCACAAA CUGAUGAG X CGAA AAACUCUU | 1236 |
| 457 | GAGUUUCU U UGUGAAAA | 101 | UGUGAAAA CUGAUGAG X CGAA AGAAACUC | 1237 |
| 458 | AGUUUCUU U GUGAAAAG | 102 | CUUUUCAC CUGAUGAG X CGAA AAGAAACU | 1238 |
| 469 | GAAAAGGU C AAGAUUAA | 103 | UUAAUCUU CUGAUGAG X CGAA ACCUUUUC | 1239 |
| 475 | GUCAAGAU U AAGACUAA | 104 | UUAGUCUU CUGAUGAG X CGAA AUCUUGAC | 1240 |
| 476 | UCAAGAUU A AGACUAAA | 105 | UUUAGUCU CUGAUGAG X CGAA AAUCUUGA | 1241 |
| 482 | UUAAGACU A AAACUUAU | 106 | AUAAGUUU CUGAUGAG X CGAA AGUCUUAA | 1242 |
| 488 | CUAAAACU U AUUGUUAC | 107 | GUAACAAU CUGAUGAG X CGAA AGUUUUAG | 1243 |
| 489 | UAAAACUU A UUGUUACC | 108 | GGUAACAA CUGAUGAG X CGAA AAGUUUUA | 1244 |
| 491 | AAACUUAU U GUUACCAU | 109 | AUGGUAAC CUGAUGAG X CGAA AUAAGUUU | 1245 |
| 494 | CUUAUUGU U ACCAUAUG | 110 | CAUAUGGU CUGAUGAG X CGAA ACAAUAAG | 1246 |
| 495 | UUAUUGUU A CCAUAUGU | 111 | ACAUAUGG CUGAUGAG X CGAA AACAAUAA | 1247 |
| 500 | GUUACCAU A UGUAUUCA | 112 | UGAAUACA CUGAUGAG X CGAA AUGGUAAC | 1248 |
| 504 | CCAUAUGU A UUCAUCUG | 113 | CAGAUGAA CUGAUGAG X CGAA ACAUAUGG | 1249 |
| 506 | AUAUGUAU U CAUCUGUU | 114 | AACAGAUG CUGAUGAG X CGAA AUACAUAU | 1250 |
| 507 | UAUGUAUU C AUCUGUUG | 115 | CAACAGAU CUGAUGAG X CGAA AAUACAUA | 1251 |
| 510 | GUAUUCAU C UGUUGGAU | 116 | AUCCAACA CUGAUGAG X CGAA AUGAAUAC | 1252 |
| 514 | UCAUCUGU U GGAUCUUG | 117 | CAAGAUCC CUGAUGAG X CGAA ACAGAUGA | 1253 |
| 519 | UGUUGGAU C UUGUAAAC | 118 | GUUUACAA CUGAUGAG X CGAA AUCCAACA | 1254 |
| 521 | UUGGAUCU U GUAAACAU | 119 | AUGUUUAC CUGAUGAG X CGAA AGAUCCAA | 1255 |
| 524 | GAUCUUGU A AACAUGAA | 120 | UUCAUGUU CUGAUGAG X CGAA ACAAGAUC | 1256 |
| 540 | AAAGGGCU U UAUUUUCA | 121 | UGAAAAUA CUGAUGAG X CGAA AGCCCUUU | 1257 |
| 541 | AAGGGCUU U AUUUUCAA | 122 | UUGAAAAU CUGAUGAG X CGAA AAGCCCUU | 1258 |
| 542 | AGGGCUUU A UUUUCAAA | 123 | UUUGAAAA CUGAUGAG X CGAA AAAGCCCU | 1259 |
| 544 | GGCUUUAU U UUCAAAAA | 124 | UUUUUGAA CUGAUGAG X CGAA AUAAAGCC | 1260 |
| 545 | GCUUUAUU U UCAAAAAU | 125 | AUUUUUGA CUGAUGAG X CGAA AAUAAAGC | 1261 |
| 546 | CUUUAUUU U CAAAAAUU | 126 | AAUUUUUG CUGAUGAG X CGAA AAAUAAAG | 1262 |
| 547 | UUUAUUUU C AAAAAUUA | 127 | UAAUUUUU CUGAUGAG X CGAA AAAAUAAA | 1263 |
| 554 | UCAAAAAU U AACUUCAA | 128 | UUGAAGUU CUGAUGAG X CGAA AUUUUUGA | 1264 |
| 555 | CAAAAAUU A ACUUCAAA | 129 | UUUGAAGU CUGAUGAG X CGAA AAUUUUUG | 1265 |
| 559 | AAUUAACU U CAAAAUAA | 130 | UUAUUUUG CUGAUGAG X CGAA AGUUAAUU | 1266 |
| 560 | AUUAACUU C AAAAUAAG | 131 | CUUAIUUU CUGAUGAG X CGAA AAGUUAAU | 1267 |
| 566 | UUCAAAAU A AGUGUAUA | 132 | UAUACACU CUGAUGAG X CGAA AUUUUGAA | 1268 |
| 572 | AUAAGUGU A UAAAAUGC | 133 | GCAUUUUA CUGAUGAG X CGAA ACACUUAU | 1269 |
| 574 | AAGUGUAU A AAAUGCAA | 134 | UUGCAUUU CUGAUGAG X CGAA AUACACUU | 1270 |
| 587 | GCAACUGU U GAUUUCCU | 135 | AGGAAAUC CUGAUGAG X CGAA ACAGUUGC | 1271 |
| 591 | CUGUUGAU U UCCUCAAC | 136 | GUUGAGGA CUGAUGAG X CGAA AUCAACAG | 1272 |
| 592 | UGUUGAUU U CCUCAACA | 137 | UGUUGAGG CUGAUGAG X CGAA AAUCAACA | 1273 |
| 593 | GUUGAUUU C CUCAACAU | 138 | AUGUUGAG CUGAUGAG X CGAA AAAUCAAC | 1274 |
| 596 | GAUUUCCU C AACAUGGC | 139 | GCCAUGUU CUGAUGAG X CGAA AGGAAAUC | 1275 |
| 606 | ACAUGGCU C ACAAAUUU | 140 | AAAUUUGU CUGAUGAG X CGAA AGCCAUGU | 1276 |
| 613 | UCACAAAU U UCUAUCCC | 141 | GGGAUAGA CUGAUGAG X CGAA AUUUGUGA | 1277 |
| 614 | CACAAAUU U CUAUCCCA | 142 | UGGGAUAG CUGAUGAG X CGAA AAUUUGUG | 1278 |
| 615 | ACAAAUUU C UAUCCCAA | 143 | UUGGGAUA CUGAUGAG X CGAA AAAUUUGU | 1279 |
| 617 | AAAUUUCU A UCCCAAAU | 144 | AUUUGGGA CUGAUGAG X CGAA AGAAAUUU | 1280 |
| 619 | AUUUCUAU C CCAAAUCU | 145 | AGAUUUGG CUGAUGAG X CGAA AUAGAAAU | 1281 |
| 626 | UCCCAAAU C UUUUCUGA | 146 | UCAGAAAA CUGAUGAG X CGAA AUUUGGGA | 1282 |
| 628 | CCAAAUCU U UUCUGAAG | 147 | CUUCAGAA CUGAUGAG X CGAA AGAUUUGG | 1283 |
| 629 | CAAAUCUU U UCUGAAGA | 148 | UCUUCAGA CUGAUGAG X CGAA AAGAUUUG | 1284 |
| 630 | AAAUCUUU U CUGAAGAU | 149 | AUCUUCAG CUGAUGAG X CGAA AAAGAUUU | 1285 |
| 631 | AAUCUUUU C UGAAGAUG | 150 | CAUCUUCA CUGAUGAG X CGAA AAAAGAUU | 1286 |
| 646 | UGAAGAGU U UAGUUUUA | 151 | UAAAACUA CUGAUGAG X CGAA ACUCUUCA | 1287 |
| 647 | GAAGAGUU U AGUUUUAA | 152 | UUAAAACU CUGAUGAG X CGAA AACUCUUC | 1288 |
| 648 | AAGAGUUU A GUUUUAAA | 153 | UUUAAAAC CUGAUGAG X CGAA AAACUCUU | 1289 |
| 651 | AGUUUAGU U UUAAAACU | 154 | AGUUUUAA CUGAUGAG X CGAA ACUAAACU | 1290 |
| 652 | GUUUAGUU U UAAAACUG | 155 | CAGUUUUA CUGAUGAG X CGAA AACUAAAC | 1291 |
| 653 | UUUAGUUU U AAAACUGC | 156 | GCAGUUUU CUGAUGAG X CGAA AAACUPAA | 1292 |
| 654 | UUAGUUUU A AAACUGCA | 157 | UGCAGUUU CUGAUGAG X CGAA AAAACUAA | 1293 |
| 675 | CAACAAGU U CACUUCAU | 158 | AUGAAGUG CUGAUGAG X CGAA ACUUGUUG | 1294 |
| 676 | AACAAGUU C ACUUCAUA | 159 | UAUGAAGU CUGAUGAG X CGAA AACUUGUU | 1295 |
| 680 | AGUUCACU U CAUAUAUA | 160 | UAUAUAUG CUGAUGAG X CGAA AGUGAACU | 1296 |
| 681 | CUUCACUU C AUAUAUAA | 162 | UUAUAUAU CUGAUGAG X CGAA AAGUGAAC | 1297 |
| 684 | CACUUCAU A UAUAAAGC | 162 | GCUUUAUA CUGAUGAG X CGAA AUGAAGUG | 1298 |
| 686 | CUUCAUAU A UAAAGCAU | 163 | AUGCUUUA CUGAUGAG X CGAA AUAUGAAG | 1299 |
| 688 | UCAUAUAU A AAGCAUUA | 164 | UAAUGCUU CUGAUGAG X CGAA AUAUAUGA | 1300 |
| 695 | UAAAGCAU U AUUUUUAC | 165 | GUAAAAAU CUGAUGAG X CGAA AUGCUUUA | 1301 |
| 696 | AAAGCAUU A UUUUUACU | 266 | AGUAAAAA CUGAUGAG X CGAA AAUGCUUU | 1302 |
| 698 | AGCAUUAU U UUUACUCU | 167 | AGAGUAAA CUGAUGAG X CGAA AUAAUGCU | 1303 |
| 699 | GCAUUAUU U UUACUCUU | 168 | AAGAGUAA CUGAUGAG X CGAA AAUAAUGC | 1304 |
| 700 | CAUUAUUU U UACUCUUU | 169 | AAAGAGUA CUGAUGAG X CGAA AAAUAAUG | 1305 |
| 701 | AUUAUUUU U ACUCUUUU | 170 | AAAAGAGU CUGAUGAG X CGAA AAAAUAAU | 1306 |
| 702 | UUAUUUUU A CUCUUUUG | 171 | CAAAAGAG CUGAUGAG X CGAA AAAAAUAA | 1307 |
| 705 | UUUUUACU C UUUUGAGG | 172 | CCUCAAAA CUGAUGAG X CGAA AGUAAAAA | 1308 |
| 707 | UUUACUCU U UUGAGGUG | 173 | CACCUCAA CUGAUGAG X CGAA AGAGUAAA | 1309 |
| 708 | UUACUCUU U UGAGGUGA | 174 | UCACCUCA CUGAUGAG X CGAA AAGAGUAA | 1310 |
| 709 | UACUCUUU U GAGGUGAA | 175 | UUCACCUC CUGAUGAG X CGAA AAAGAGUA | 1311 |
| 719 | AGGUGAAU A UAAUUUAU | 176 | AUAAAUUA CUGAUGAG X CGAA AUUCACCU | 1312 |
| 721 | GUGAAUAU A AUUUAUAU | 177 | AUAUAAAU CUGAUGAG X CGAA AUAUUCAC | 1313 |
| 724 | AAUAUAAU U UAUAUUAC | 178 | GUAAUAUA CUGAUGAG X CGAA AUUAUAUU | 1314 |
| 725 | AUAUAAUU U AUAUUACA | 179 | UGUAAUAU CUGAUGAG X CGAA AAUUAUAU | 1315 |
| 726 | UAUAAUUU A UAUUACAA | 180 | CAUUGUAA CUGAUGAG X CGAA AAAUUAUA | 1316 |
| 728 | UAAUUUAU A UUACAAUG | 181 | CAUUGUAA CUGAUGAG X CGAA AUAAAUUA | 1317 |
| 730 | AUUUAUAU U ACAAUGUA | 182 | UACAUUGU CUGAUGAG X CGAA AUAUAAAU | 1318 |
| 731 | UUUAUAUU A CAAUGUAA | 183 | UUACAUUG CUGAUGAG X CGAA AAUAUAAA | 1319 |
| 738 | UACAAUGU A AAAGCUUC | 184 | GAGGCUUU CUGAUGAG X CGAA ACAUUGUA | 1320 |
| 745 | UAAAAGCU U CUUUAAUA | 185 | UAUUAAAG CUGAUGAG X CGAA AGCUUUUA | 1321 |
| 746 | AAAAGCUU C UUUAAUAC | 186 | GUAUUAAA CUGAUGAG X CGAA AAGCUUUU | 1322 |
| 748 | AAGCUUCU U UAAUACUA | 187 | UAGUAUUA CUGAUGAG X CGAA AGAAGCUU | 1323 |
| 749 | AGCUUCUU U AAUACUAA | 188 | UUAGUAUU CUGAUGAG X CGAA AAGAAGCU | 1324 |
| 750 | GCUUCUUU A AUACUAAG | 189 | CUUAGUAU CUGAUGAG X CGAA AAAGAAGC | 1325 |
| 753 | UCUUUAAU A CUAAGUAU | 190 | AUACUUAG CUGAUGAG X CGAA AUUAAAGA | 1326 |
| 756 | UUAAUACU A AGUAUUUU | 191 | AAAAUACU CUGAUGAG X CGAA AGUAUUAA | 1327 |
| 760 | UACUAAGU A UUUUUCAG | 192 | CUGAAAAA CUGAUGAG X CGAA ACUUAGUA | 1328 |
| 762 | CUAAGUAU U UUUCAGGU | 193 | ACCUGAAA CUGAUGAG X CGAA AUACUUAG | 1329 |
| 763 | UAAGUAUU U UUCAGGUC | 194 | GACCUGAA CUGAUGAG X CGAA AAUACUUA | 1330 |
| 764 | AAGUAUUU U UCAGGUCU | 195 | AGACCUGA CUGAUGAG X CGAA AAAUACUU | 1331 |
| 765 | AGUAUUUU U CAGGUCUU | 196 | AAGACCUG CUGAUGAG X CGAA AAAAUACU | 1332 |
| 766 | GUAUUUUU C AGGUCUUC | 197 | GAAGACCU CUGAUGAG X CGAA AAAAAUAC | 1333 |
| 771 | UUUCAGGU C UUCACCAA | 198 | UUGGUGAA CUGAUGAG X CGAA ACCUGAAA | 1334 |
| 773 | UCAGGUCU U CACCAAGU | 199 | ACUUGGUG CUGAUGAG X CGAA AGACCUGA | 1335 |
| 774 | CAGGUCUU C ACCAAGUA | 200 | UACUUGGU CUGAUGAG X CGAA AAGACCUG | 1336 |
| 782 | CACCAAGU A UCAAAGUA | 201 | UACUUUGA CUGAUGAG X CGAA ACUUGGUG | 1337 |
| 784 | CCAAGUAU C AAAGUAAU | 202 | AUUACUUU CUGAUGAG X CGAA AUACUUGG | 1338 |
| 790 | AUCAAAGU A AUAACACA | 203 | UGUGUUAU CUGAUGAG X CGAA ACUUUGAU | 1339 |
| 793 | AAAGUAAU A ACACAAAU | 204 | AUUUGUGU CUGAUGAG X CGAA AUUACUUU | 1340 |
| 809 | UGAAGUGU C AUUAUUCA | 205 | UGAAUAAU CUGAUGAG X CGAA ACACUUCA | 1341 |
| 812 | AGUGUCAU U AUUCAAAA | 206 | UUUUGAAU CUGAUGAG X CGAA AUGACACU | 1342 |
| 813 | GUGUCAUU A UUCAAAAU | 207 | AUUUUGAA CUGAUGAG X CGAA AAUGACAC | 1343 |
| 815 | GUCAUUAU U CAAAAUAG | 208 | CUAUUUUG CUGAUGAG X CGAA AUAAUGAC | 1344 |
| 816 | UCAUUAUU C AAAAUAGU | 209 | ACUAUUUU CUGAUGAG X CGAA AAUAAUGA | 1345 |
| 822 | UUCAAAAU A GUCCACUG | 210 | CAGUGGAC CUGAUGAG X CGAA AUUUUGAA | 1346 |
| 825 | AAAAUAGU C CACUGACU | 211 | AGUCAGUG CUGAUGAG X CGAA ACUAUUUU | 1347 |
| 834 | CACUGACU C CUCACAUC | 212 | GAUGUGAG CUGAUGAG X CGAA AGUCAGUG | 1348 |
| 837 | UGACUCCU C ACAUCUGU | 213 | ACAGAUGU CUGAUGAG X CGAA AGGAGUCA | 1349 |
| 842 | CCUCACAU C UGUUAUCU | 214 | AGAUAACA CUGAUGAG X CGAA AUGUGAGG | 1350 |
| 846 | ACAUCUGU U AUCUUAUU | 215 | AAUAAGAU CUGAUGAG X CGAA ACAGAUGU | 1351 |
| 847 | CAUCUGUU A UCUUAUUA | 216 | UAAUAAGA CUGAUGAG X CGAA AACAGAUG | 1352 |
| 849 | UCUGUUAU C UUAUUAUA | 217 | UAUAAUAA CUGAUGAG X CGAA AUAACAGA | 1353 |
| 851 | UGUUAUCU U AUUAUAAA | 218 | UUUAUAAU CUGAUGAG X CGAA AGAUAACA | 1354 |
| 852 | GUUAUCUU A UUAUAAAG | 219 | CUUUAUAA CUGAUGAG X CGAA AAGAUAAC | 1355 |
| 854 | UAUCUUAU U AUAAAGAA | 220 | UUCUUUAU CUGAUGAG X CGAA AUAAGAUA | 1356 |
| 855 | AUCUUAUU A UAAAGAAC | 221 | GUUCUUUA CUGAUGAG X CGAA AAUAAGAU | 1357 |
| 857 | CUUAUUAU A AAGAACUA | 222 | UAGUUCUU CUGAUGAG X CGAA AUAAUAAG | 1358 |
| 865 | AAAGAACU A UUUGUAGU | 223 | ACUACAAA CUGAUGAG X CGAA AGUUCUUU | 1359 |
| 867 | AGAACUAU U UGUAGUAA | 224 | CUACUACA CUGAUGAG X CGAA AUAGUUCU | 1360 |
| 868 | GAACUAUU U GUAGUAAC | 225 | GUUACUAC CUGAUGAG X CGAA AAUAGUUC | 1361 |
| 871 | CUAUUUGU A GUAACUAU | 226 | AUAGUUAC CUGAUGAG X CGAA ACAAAUAG | 1362 |
| 874 | UUUGUAGU A ACUAUCAG | 227 | CUGAUAGU CUGAUGAG X CGAA ACUACAAA | 1363 |
| 878 | UAGUAACU A UCAGAAUC | 228 | GAUUCUGA CUGAUGAG X CGAA AGUUACUA | 1364 |
| 880 | GUAACUAU C AGAAUCUA | 229 | UAGAUUCU CUGAUGAG X CGAA AUAGUUAC | 1365 |
| 886 | AUCAGAAU C UACAUUCU | 230 | AGAAUGUA CUGAUGAG X CGAA AUUCUGAU | 1366 |
| 888 | CAGAAUCU A CAUUCUAA | 231 | UUAGAAUG CUGAUGAG X CGAA AGAUUCUG | 1367 |
| 892 | AUCUACAU U CUAAAACA | 232 | UGUUUUAG CUGAUGAG X CGAA AUGUAGAU | 1368 |
| 893 | UCUACAUU C UAAAACAG | 233 | CUGUUUUA CUGAUGAG X CGAA AAUGUAGA | 1369 |
| 895 | UACAUUCU A AAACAGAA | 234 | UUCUGUUU CUGAUGAG X CGAA AGAAUGUA | 1370 |
| 906 | ACAGAAAU U GUAUUUUU | 235 | AAAAAUAC CUGAUGAG X CGAA AUUUCUGU | 1371 |
| 909 | GAAAUUGU A UUUUUUCU | 236 | AGAAAAAA CUGAUGAG X CGAA ACAAUUUC | 1372 |
| 911 | AAUUGUAU U UUUUCUAU | 237 | AUAGAAAA CUGAUGAG X CGAA AUACAAUU | 1373 |
| 912 | AUUGUAUU U UUUCUAUG | 238 | CAUAGAAA CUGAUGAG X CGAA AAUACAAU | 1374 |
| 913 | UUGUAUUU U UUCUAUGC | 239 | GCAUAGAA CUGAUGAG X CGAA AAAUACAA | 1375 |
| 914 | UGUAUUUU U UCUAUGCC | 240 | GGCAUAGA CUGAUGAG X CGAA AAAAUACA | 1376 |
| 915 | GUAUUUUU U CUAUGCCA | 241 | UGGCAUAG CUGAUGAG X CGAA AAAAAUAC | 1377 |
| 916 | UAUUUUUU C UAUGCCAC | 242 | GUGGCAUA CUGAUGAG X CGAA AAAAAAUA | 1378 |
| 918 | UUUUUUCU A UGCCACAU | 243 | AUGUGGCA CUGAUGAG X CGAA AGAAAAAA | 1379 |
| 927 | UGCCACAU U AACAUCUU | 244 | AAGAUGUU CUGAUGAG X CGAA AUGUGGCA | 1380 |
| 928 | GCCACAUU A ACAUCUUU | 245 | AAAGAUGU CUGAUGAG X CGAA AAUGUGGC | 1381 |
| 933 | AUUAACAU C UUUUAAAG | 246 | CUUUAAAA CUGAUGAG X CGAA AUGUUAAU | 1382 |
| 935 | UAACAUCU U UUAAAGUU | 247 | AACUUUAA CUGAUGAG X CGAA AGAUGUUA | 1383 |
| 936 | AACAUCUU U UAAAGUUG | 248 | CAACUUAA CUGAUGAG X CGAA AAGAUGUU | 1384 |
| 937 | ACAUCUUU U AAAGUUGA | 249 | UCAACUUU CUGAUGAG X CGAA AAAGAUGU | 1385 |
| 938 | CAUCUUUU A AAGUUGAU | 250 | AUCAACUU CUGAUGAG X CGAA AAAAGAUG | 1386 |
| 943 | UUUAAAGU U GAUGAGAA | 251 | UUCUCAUC CUGAUGAG X CGAA ACUUUAAA | 1387 |
| 953 | AUGAGAAU C AAGUAUGG | 252 | CCAUACUU CUGAUGAG X CGAA AUUCUCAU | 1388 |
| 958 | AAUCAAGU A UGGAAAAG | 253 | CUUUUCCA CUGAUGAG X CGAA ACUUGAUU | 1389 |
| 968 | GGAAAAGU A AGGCCAUA | 254 | UAUGGCCU CUGAUGAG X CGAA AUGGCCUU | 1390 |
| 976 | AAGGCCAT A CUCUUACA | 255 | UGUAAGAG CUGAUGAG X CGAA AGUAUGGC | 1392 |
| 979 | GCCAUACU C UUACAUAA | 256 | UUAUGUAA CUGAUGAG X CGAA AGUAUGGC | 1393 |
| 981 | CAUACUCU U ACAUAAUA | 257 | UAUUAUGU CUGAUGAG X CGAA AAGAGUAU | 1394 |
| 982 | AUACUCUU A CAUAAUAA | 258 | UUAUUAUG CUGAUGAG X CGAA AAGAGUAU | 1394 |
| 986 | UCUUACAU A AUAAAAUU | 259 | AAUUUUAU CUGAUGAG X CGAA AUGUAAGA | 1395 |
| 989 | UACAUAAU A AAAUUCCU | 260 | AGGAAUUU CUGAUGAG X CGAA AUUAUGUA | 1396 |
| 994 | AAUAAAAU U CCUUUUAA | 261 | UUAAAAGG CUGAUGAG X CGAA AUUUUAUU | 1397 |
| 995 | AUAAAAUU C CUUUUAAG | 262 | CUUAAAAG CUGAUGAG X CGAA AAUUUUAU | 1398 |
| 998 | AAAUUCCU U UUAAGUAA | 263 | UUACUUAA CUGAUGAG X CGAA AGGAAUUU | 1399 |
| 999 | AAUUCCUU U UAAGUAAU | 264 | AUUACUUA CUGAUGAG X CGAA AAGGAAUU | 1400 |
| 1000 | AUUCCUUU U AAGUAAUU | 265 | AAUUACUU CUGAUGAG X CGAA AAAGGAAU | 1401 |
| 1001 | UUCCUUUU A AGUAAUUU | 266 | AAAUUACU CUGAUGAG X CGAA AAAAGGAA | 1402 |
| 1005 | UUUUAAGU A AUUUUUUC | 267 | GAAAAAAU CUGAUGAG X CGAA ACUUAAAA | 1403 |
| 1008 | UAAGUAAU U UUUUCAAA | 268 | UUUGAAAA CUGAUGAG X CGAA AUUACUUA | 1404 |
| 1009 | AAGUAAUU U UUUCAAAG | 269 | CUUUGAAA CUGAUGAG X CGAA AAUUACUU | 1405 |
| 1010 | AGUAAUUU U UUCAAAGA | 270 | UCUUUGAA CUGAUGAG X CGAA AAAUUACU | 1406 |
| 1011 | GUAAUUUU U UCAAAGAA | 271 | UUCUUUGA CUGAUGAG X CGAA AAAAUUAC | 1407 |
| 1012 | UAAUUUUU U CAAAGAAU | 272 | AUUCUUUG CUGAUGAG X CGAA AAAAAUUA | 1408 |
| 1013 | AAUUUUUU C AAAGAAUC | 273 | GAUUCUUU CUGAUGAG X CGAA AAAAAAUU | 1409 |
| 1021 | CAAAGAAU C ACAGAAUU | 274 | AAUUCUGU CUGAUGAG X CGAA AUUCUUUG | 1410 |
| 1029 | CACAGAAU U CUAGUACA | 275 | UGUACUAG CUGAUGAG X CGAA AUUCUGUG | 1411 |
| 1030 | ACAGAAUU A GUACAUGU | 276 | ACAUGUAC CUGAUGAG X CGAA AAUUCUGU | 1412 |
| 1032 | AGAAUUCU A GUACAUGU | 277 | ACAUGUAC CUGAUGAG X CGAA AGAAUUCU | 1413 |
| 1035 | AUUCUAGU A CAUGUAGG | 278 | CCUACAUG CUGAUGAG X CGAA ACUAGAAU | 1414 |
| 1041 | GUACAUGU A GGUAAAUC | 279 | GAUUUACC CUGAUGAG X CGAA ACAUGUAC | 1415 |
| 1045 | AUGUAGGU A AAUCAUAA | 280 | UUAUGAUU CUGAUGAG X CGAA ACCUACAU | 1416 |
| 1049 | AGGUAAAU C AUAAAUCU | 281 | AGAUUUAU CUGAUGAG X CGAA AUUUACCU | 1417 |
| 1052 | UAAAUCAU A AAUCUGUU | 282 | AACAGAUU CUGAUGAG X CGAA AUGAUUUA | 1418 |
| 1056 | UCAUAAAU C UGUUCUAA | 283 | UUAGAACA CUGAUGAG X CGAA AUUUAUGA | 1419 |
| 1060 | AAAUCUGU U CUAAGACA | 284 | UGUCUUAG CUGAUGAG X CGAA ACAGAUUU | 1420 |
| 1061 | AAUCUGUU C UAAGACAU | 285 | AUGUCUUA CUGAUGAG X CGAA AACAGAUU | 1421 |
| 1063 | UCUGUUCU A AGACAUAU | 286 | AUAUGUCU CUGAUGAG X CGAA AGAACAGA | 1422 |
| 1070 | UAAGACAU A UGAUCAAC | 287 | GUUGAUCA CUGAUGAG X CGAA AUGUCUUA | 1423 |
| 1075 | CAUAUGAU C AACAGAUG | 288 | CAUCUGUU CUGAUGAG X CGAA AUCAUAUG | 1424 |
| 1096 | CUGGUGGU U AAUAUGUG | 289 | CACAUAUU CUGAUGAG X CGAA ACCACCAG | 1425 |
| 1097 | UGGUGGUU A AUAUGUGA | 290 | UCACAUAU CUGAUGAG X CGAA AACCACCA | 1426 |
| 1100 | UGGUUAAU A UGUGACAG | 291 | CUGUCACA CUGAUGAG X CGAA AUUAACCA | 1427 |
| 1115 | AGUGAGAU U AGUCAUAU | 292 | AUAUGACU CUGAUGAG X CGAA AUCUCACU | 1428 |
| 1116 | GUGAGAUU A GUCAUAUC | 293 | GAUAUGAC CUGAUGAG X CGAA AAUCUCAC | 1429 |
| 1119 | AGAUUAGU C AUAUCACU | 294 | AGUGAUAU CUGAUGAG X CGAA ACUAAUCU | 1430 |
| 1122 | UUAGUCAU A UCACUAAU | 295 | AUUAGUGA CUGAUGAG X CGAA AUGACUAA | 1431 |
| 1124 | AGUCAUAU C ACUAAUAU | 296 | AUAUUAGU CUGAUGAG X CGAA AUAUGACU | 1432 |
| 1128 | AUAUCACU A AUAUACUA | 297 | UAGUAUAU CUGAUGAG X CGAA AGUGAUAU | 1433 |
| 1131 | UCACUAAU A UACUAACA | 298 | UGUUAGUA CUGAUGAG X CGAA AUUAGUGA | 1434 |
| 1133 | ACUAAUAU A CUAACAAC | 299 | GUUGUUAG CUGAUGAG X CGAA AUAUUAGU | 1435 |
| 1136 | AAUAUACU A ACAACAGA | 300 | UCUGUUGU CUGAUGAG X CGAA AGUAUAUU | 1436 |
| 1147 | AACAGAAU C UAAUCUUC | 301 | GAAGAUUA CUGAUGAG X CGAA AUUCUGUU | 1437 |
| 1149 | CAGAAUCU A AUCUUCAU | 302 | AUGAAGAU CUGAUGAG X CGAA AGAUUCUG | 1438 |
| 1152 | AAUCUAAU C UUCAUUUA | 303 | UAAAUGAA CUGAUGAG X CGAA AUUAGAUU | 1439 |
| 1154 | UCUAAUCU U CAUUUAAG | 304 | CUUAAAUG CUGAUGAG X CGAA AGAUUAGA | 1440 |
| 1155 | CUAAUCUU C AUUUAAGG | 305 | CCUUAAAU CUGAUGAG X CGAA AAGAUUAG | 1441 |
| 1158 | AUCUUCAU U UAAGGCAC | 306 | GUGCCUUA CUGAUGAG X CGAA AUGAAGAU | 1442 |
| 1159 | UCUUCAUU U AAGGCACU | 307 | AGUGCCUU CUGAUGAG X CGAA AAUGAAGA | 1443 |
| 1160 | CUUCAUUU A AGGCACUG | 308 | CAGUGCCU CUGAUGAG X CGAA AAAUGAAG | 1444 |
| 1170 | GGCACUGU A GUGAAUUA | 309 | UAAUUCAC CUGAUGAG X CGAA ACAGUGCC | 1445 |
| 1177 | UAGUGAAU U AUCUGAGC | 310 | GCUCAGAU CUGAUGAG X CGAA AUUCACUA | 1446 |
| 1178 | AGUGAAUU A UCUGAGCU | 311 | AGCUCAGA CUGAUGAG X CGAA AAUUCACU | 1447 |
| 1180 | UGAAUUAU C UGAGCUAG | 312 | CUAGCUCA CUGAUGAG X CGAA AUAAUUCA | 1448 |
| 1187 | UCUGAGCU A GAGUUACC | 313 | GGUAACUC CUGAUGAG X CGAA AGCUCAGA | 1449 |
| 1192 | GCUAGAGU U ACCUAGCU | 314 | AGCUAGGU CUGAUGAG X CGAA ACUCUAGC | 1450 |
| 1193 | CUAGAGUU A CCUAGCUU | 315 | AAGCUAGG CUGAUGAG X CGAA AACUCUAG | 1451 |
| 1197 | AGUUACCU A GCUUACCA | 316 | GCUUACCA CUGAUGAG X CGAA AGGUAACU | 1452 |
| 1201 | ACCUAGCU U ACCAUACU | 317 | AGUAUGGU CUGAUGAG X CGAA AGCUAGGU | 1453 |
| 1202 | CCUAGCUU A CCAUACUA | 313 | UAGUAUGG CUGAUGAG X CGAA AAGCUAGG | 1454 |
| 1207 | CUUACCAU A CUAUAUCU | 319 | AGAUAUAG CUGAUGAG X CGAA AUGGUAAG | 1455 |
| 1210 | ACCAUACU A UAUCUUUG | 320 | CAAAGAUA CUGAUGAG X CGAA AGUAUGGU | 1456 |
| 1212 | CAUACUAU A UCUUUGGA | 321 | UCCAAAGA CUGAUGAG X CGAA AUAGUAUG | 1457 |
| 1214 | UACUAUAU C UUUGGAAU | 322 | AUUCCAAA CUGAUGAG X CGAA AUAUAGUA | 1458 |
| 1216 | CUAUAUCU U UGGAAUCA | 323 | UGAUUCCA CUGAUGAG X CGAA AGAUAUAG | 1459 |
| 1217 | UAUAUCUU U GGAAUCAU | 324 | AUGAUUCC CUGAUGAG X CGAA AAGAUAUA | 1460 |
| 1223 | UUUGGAAU C AUGAAACC | 325 | GGUUUCAU CUGAUGAG X CGAA AUUCCAAA | 1461 |
| 1233 | UGAAACCU U AAGACUUC | 326 | GAAGUCUU CUGAUGAG X CGAA AGGUUUCA | 1462 |
| 1234 | GAAACCUU A AGACUUCA | 327 | UGAAGUCU CUGAUGAG X CGAA AAGGUUUC | 1463 |
| 1240 | UUAAGACU U CAGAAUGA | 328 | UCAUUCUG CUGAUGAG X CGAA AGUCUUAA | 1464 |
| 1241 | UAAGACUU C AGAAUGAU | 329 | AUCAUUCU CUGAUGAG X CGAA AAGUCUUA | 1465 |
| 1250 | AGAAUGAU U UUGCAGGU | 330 | ACCUGCAA CUGAUGAG X CGAA AUCAUUCU | 1466 |
| 1251 | GAAUGAUU U UGCAGGUU | 331 | AACCUGCA CUGAUGAG X CGAA AAUCAUUC | 1467 |
| 1252 | AAUGAUUU U GCAGGUUG | 332 | CAACCUGC CUGAUGAG X CGAA AAAUCAUU | 1468 |
| 1259 | UUGCAGGU U GUCUUCCA | 333 | UGGAAGAC CUGAUGAG X CGAA ACCUGCAA | 1469 |
| 1262 | CAGGUUGU C UUCCAUUC | 334 | GAAUGGAA CUGAUGAG X CGAA ACAACCUG | 1470 |
| 1264 | GGUUGUCU U CCAUUCCA | 335 | UGGAAUGG CUGAUGAG X CGAA AGACAACC | 1471 |
| 1265 | GUUGUCUU C CAUUCCAG | 336 | CUGGAAUG CUGAUGAG X CGAA AAGACAAC | 1472 |
| 1269 | UCUUCCAU U CCAGCCUA | 337 | UAGGCUGG CUGAUGAG X CGAA AUGGAAGA | 1473 |
| 1270 | CUUCCAUU C CAGCCUAA | 338 | UUAGGCUG CUGAUGAG X CGAA AAUGGAAG | 1474 |
| 1277 | UCCAGCCU A ACAUCCAA | 339 | UUGGAUGU CUGAUGAG X CGAA AGGCUGGA | 1475 |
| 1282 | CCUAACAU C CAAUGCAG | 340 | CUGCAUUG CUGAUGAG X CGAA AUGUUAGG | 1476 |
| 1302 | AGGAAAAU A AAAGAUUU | 341 | AAAUCUUU CUGAUGAG X CGAA AGGCUGGA | 1477 |
| 1309 | UAAAAGAU U UCCAGUGA | 342 | UCACUGGA CUGAUGAG X CGAA AUCUUUUA | 1478 |
| 1310 | AAAAGAUU U CCAGUGAC | 343 | GUCACUGG CUGAUGAG X CGAA AAUCUUUU | 1479 |
| 1311 | AAAGAUUU C CAGUGACA | 344 | UGUCACUG CUGAUGAG X CGAA AAAUCUUU | 1480 |
| 1327 | AGAAAAAU A UAUUAUCU | 345 | AGAUAAUA CUGAUGAG X CGAA AUUUUUCU | 1481 |
| 1329 | AAAAAUAU A UUAUCUCA | 346 | UGAGAUAA CUGAUGAG X CGAA AUAUUUUU | 1482 |
| 1331 | AAAUAUAU U AUCUCAAG | 347 | CUUGAGAU CUGAUGAG X CGAA AUAUAUUU | 1483 |
| 1332 | AAUAUAUU A UCUCAAGU | 348 | ACUUGAGA CUGAUGAG X CGAA AAUAUAUU | 1484 |
| 1334 | UAUAUUAU C UCAAGUAU | 349 | AUACUUGA CUGAUGAG X CGAA AUAAUAUA | 1485 |
| 1336 | UAUUAUCU C AAGUAUUU | 350 | AAAUACUU CUGAUGAG X CGAA AGAUAAUA | 1486 |
| 1341 | UCUCAAGU A UUUUUUAA | 351 | UUAAAAAA CUGAUGAG X CGAA ACUUGAGA | 1487 |
| 1343 | UCAAGUAU U UUUUAAAA | 352 | UUUUAAAA CUGAUGAG X CGAA AUACUUGA | 1488 |
| 1344 | CAAGUAUU U UUUAAAAA | 353 | UUUUUAAA CUGAUGAG X CGAA AAUACUUG | 1489 |
| 1345 | AAGUAUUU U UUAAAAAU | 354 | AUUUUUAA CUGAUGAG X CGAA AAAUACUU | 1490 |
| 1346 | AGUAUUUU U UAAAAAUA | 355 | UAUUUUUA CUGAUGAG X CGAA AAAAUACU | 1491 |
| 1347 | GUAUUUUU U AAAAAUAU | 356 | AUAUUUUU CUGAUGAG X CGAA AAAAAUAC | 1492 |
| 1348 | UAUUUUUU A AAAAUAUA | 357 | UAUAUUUU CUGAUGAG X CGAA AAAAAAUA | 1493 |
| 1354 | UUAAAAAU A UAUGAAUU | 358 | AAUUCAUA CUGAUGAG X CGAA AUUUUUAA | 1494 |
| 1356 | AAAAAUAU A UGAAUUCU | 359 | AGAAUUCA CUGAUGAG X CGAA AUAUUUUU | 1495 |
| 1362 | AUAUGAAU U CUCUCUCC | 360 | GGAGAGAG CUGAUGAG X CGAA AUUCAUAU | 1496 |
| 1363 | UAUGAAUU C UCUCUCCA | 361 | UGGAGAGA CUGAUGAG X CGAA AAUUCAUA | 1497 |
| 1365 | UGAAUUCU C UCUCCAAA | 362 | UUUGGAGA CUGAUGAG X CGAA AGAAUUCA | 1498 |
| 1367 | AAUUCUCU C UCCAAAUA | 363 | UAUUUGGA CUGAUGAG X CGAA AGAGAAUU | 1499 |
| 1369 | UUCUCUCU C CAAAUAUU | 364 | AAUAUUUG CUGAUGAG X CGAA AGAGAGAA | 1500 |
| 1375 | CUCCAAAU A UUAACUAA | 365 | UUAGUUAA CUGAUGAG X CGAA AUUUGGAG | 1501 |
| 1377 | CCAAAUAU U AACUAAUU | 366 | AAUUAGUU CUGAUGAG X CGAA AUAUUUGG | 1502 |
| 1378 | CAAAUAUU A ACUAAUUA | 367 | UAAUUAGU CUGAUGAG X CGAA AAUAUUUG | 1503 |
| 1382 | UAUUAACU A AUUAUUAG | 388 | CUAAUAAU CUGAUGAG X CGAA AGUUAAUA | 1504 |
| 1385 | UAACUAAU U AUUAGAUU | 369 | AAUCUAAU CUGAUGAG X CGAA AUUAGUUA | 1505 |
| 1386 | AACUAAUU A UUAGAUUA | 370 | UAAUCUAA CUGAUGAG X CGAA AAUUAGUU | 1506 |
| 1388 | CUAAUUAU U AGAUUAUA | 371 | UAUAAUCU CUGAUGAG X CGAA AUAAUUAG | 1507 |
| 1389 | UAAUUAUU A GAUUAUAU | 372 | AUAUAAUC CUGAUGAG X CGAA AAUAAUUA | 1508 |
| 1393 | UAUUAGAU U AUAUUUUG | 373 | CAAAAUAU CUGAUGAG X CGAA AUCUAAUA | 1509 |
| 1394 | AUUAGAUU A UAUUUUGA | 374 | UCAAAAUA CUGAUGAG X CGAA AAUCUAAU | 1510 |
| 1396 | UAGAUUAU A UUUUGAAA | 375 | UUUCAAAA CUGAUGAG X CGAA AUAAUCUA | 1511 |
| 1398 | GAUUAUAU U UUGAAAUG | 376 | CAUUUCAA CUGAUGAG X CGAA AUAUAAUC | 1512 |
| 1399 | AUUAUAUU U UGAAAUGA | 377 | UCAUUUCA CUGAUGAG X CGAA AAUAUAAU | 1513 |
| 1400 | UUAUAUUU U GAAAUGAA | 378 | UUCAUUUC CUGAUGAG X CGAA AAAUAUAA | 1514 |
| 1411 | AAUGAACU U GUUGGCCC | 379 | GGGCCAAC CUGAUGAG X CGAA AGUUCAUU | 1515 |
| 1414 | GAACUUGU U GGCCCAUC | 380 | GAUGGGCC CUGAUGAG X CGAA ACAAGUUC | 1516 |
| 1422 | UGGCCCAU C UAUUACAU | 381 | AUGUAAUA CUGAUGAG X CGAA AUGGGCCA | 1517 |
| 1424 | GCCCAUCU A UUACAUCU | 382 | AGAUGUAA CUGAUGAG X CGAA AGAUGGGC | 1518 |
| 1426 | CCAUCUAU U ACAUCUAC | 383 | GUAGAUGU CUGAUGAG X CGAA AUAGAUGG | 1519 |
| 1427 | CAUCUAUU A CAUCUACA | 384 | UGUAGAUG CUGAUGAG X CGAA AAUAGAUG | 1520 |
| 1431 | UAUUACAU C UACAGCUG | 385 | CAGCUGUA CUGAUGAG X CGAA AUGUAAUA | 1521 |
| 1433 | UUACAUCU A CAGCUGAC | 386 | GUCAGCUG CUGAUGAG X CGAA AGAUGUAA | 1522 |
| 1445 | CUGACCCU U GAACAUGG | 387 | CCAUGUUC CUGAUGAG X CGAA AGGGUCAG | 1523 |
| 1458 | AUGGGGGU U AGGGGAGC | 388 | GCUCCCCU CUGAUGAG X CGAA ACCCCCAU | 1524 |
| 1459 | UGGGGGUU A GGGGAGCU | 389 | AGCUCCCC CUGAUGAG X CGAA AACCCCCA | 1525 |
| 1474 | CUGACAAU U CGUGGGUC | 390 | GACCCACG CUGAUGAG X CGAA AUUGUCAG | 1526 |
| 1475 | UGACAAUU C GUGGGUCC | 391 | GGACCCAC CUGAUGAG X CGAA AAUUGUCA | 1527 |
| 1482 | UCGUGGGU C CGCAAAAU | 392 | AUUUUGCG CUGAUGAG X CGAA ACCCACGA | 1528 |
| 1491 | CGCAAAAU C UUAACUAC | 393 | GUAGUUAA CUGAUGAG X CGAA AUUUUGCG | 1529 |
| 1493 | CAAAAUCU U AACUACCU | 394 | AGGUAGUU CUGAUGAG X CGAA AGAUUUUG | 1530 |
| 1494 | AAAAUCUU A ACUACCUA | 395 | UAGGUAGU CUGAUGAG X CGAA AAGAUUUU | 1531 |
| 1498 | UCUUAACU A CCUAAUAG | 396 | CUAUUAGG CUGAUGAG X CGAA AGUUAAGA | 1532 |
| 1502 | AACUACCU A AUAGCCUA | 397 | UAGGCUAU CUGAUGAG X CGAA AGGUAGUU | 1533 |
| 1505 | UACCUAAU A GCCUACUA | 398 | UAGUAGGC CUGAUGAG X CGAA AUUAGGUA | 1534 |
| 1510 | AAUAGCCU A CUAUUGAC | 399 | GUCAAUAG CUGAUGAG X CGAA AGGCUAUU | 1535 |
| 1513 | AGCCUACU A UUGACCAU | 400 | AUGGUCAA CUGAUGAG X CGAA AGUAGGCU | 1536 |
| 1515 | CCUACUAU U GACCAUAA | 401 | UUAUGGUC CUGAUGAG X CGAA AUAGUAGG | 1537 |
| 1522 | UUGACCAU A AACCUUAC | 402 | GUAAGGUU CUGAUGAG X CGAA AUGGUCAA | 1538 |
| 1528 | AUAAACCU U ACUGAUAA | 403 | UUAUCAGU CUGAUGAG X CGAA AGGUUUAU | 1539 |
| 1529 | UAAACCUU A CUGAUAAC | 404 | GUUAUCAG CUGAUGAG X CGAA AAGGUUUA | 1540 |
| 1535 | UUACUGAU A ACAUAAAC | 405 | GUUUAUGU CUGAUGAG X CGAA AUCAGUAA | 1541 |
| 1540 | GAUAACAU A AACAGUAA | 406 | UUACUGUU CUGAUGAG X CGAA AUGUUAUC | 1542 |
| 1547 | UAAACAGU A AAUUAACA | 407 | UGUUAAUU CUGAUGAG X CGAA ACUGUUUA | 1543 |
| 1551 | CAGUAAAU U AACACAUA | 408 | UAUGUGUU CUGAUGAG X CGAA AUUUACUG | 1544 |
| 1552 | AGUAAAUU A ACACAUAU | 409 | AUAUGUGU CUGAUGAG X CGAA AAUUUACU | 1545 |
| 1559 | UAACACAU A UUUUGCGU | 410 | ACGCAAAA CUGAUGAG X CGAA AUGUGUUA | 1546 |
| 1561 | ACACAUAU U UUGCGUGU | 411 | ACACGCAA CUGAUGAG X CGAA AUAUGUGU | 1547 |
| 1562 | CACAUAUU U UGCGUGUU | 412 | AACACGCA CUGAUGAG X CGAA AAUAUGUG | 1548 |
| 1563 | ACAUAUUU U GCGUGUUA | 413 | UAACACGC CUGAUGAG X CGAA AAAUAUGU | 1549 |
| 1570 | UUGCGUGU U AUAUGUAU | 414 | AUACAUAU CUGAUGAG X CGAA ACACGCAA | 1550 |
| 1571 | UGCGUGUU A UAUGUAUU | 415 | AAUACAUA CUGAUGAG X CGAA AACACGCA | 1551 |
| 1573 | CGUGUUAU A UGUAUUAU | 416 | AUAAUACA CUGAUGAG X CGAA AUAACACG | 1552 |
| 1577 | UUAUAUGU A UUAUACAC | 417 | GUGUAUAA CUGAUGAG X CGAA ACAUAUAA | 1553 |
| 1579 | AUAUGUAU U AUACACUA | 418 | UAGUGUAU CUGAUGAG X CGAA AUACAUAU | 1554 |
| 1580 | UAUGUAUU A UACACUAU | 419 | AUAGUGUA CUGAUGAG X CGAA AAUACAUA | 1555 |
| 1582 | UGUAUUAU A CACUAUAU | 420 | AUAGUGUA CUGAUGAG X CGAA AUAAUACA | 1556 |
| 1587 | UAUACACU A UAUUCCUA | 421 | UAGGAAUA CUGAUGAG X CGAA AGUGUAUA | 1557 |
| 1589 | UACACUAU A UUCCUACA | 422 | UGUAGGAA CUGAUGAG X CGAA AUAGUGUA | 1558 |
| 1591 | CACUAUAU U CCUACAAU | 423 | AUUGUAGG CUGAUGAG X CGAA AUAUAGUG | 1559 |
| 1592 | ACUAUAUU C CUACAAUA | 424 | UAUUGUAG CUGAUGAG X CGAA AAUAUAGU | 1560 |
| 1595 | AUAUUCCU A CAAUAAAG | 425 | CUUUAUUG CUGAUGAG X CGAA AGGAAUAU | 1561 |
| 1600 | CCUACAAU A AAGUAAGC | 426 | GCUUACUU CUGAUGAG X CGAA AUUGUAGG | 1562 |
| 1605 | AAUAAAGU A AGCUAGAG | 427 | CUCUAGCU CUGAUGAG X CGAA ACUUUAUU | 1563 |
| 1610 | AGUAAGCU A GAGAAAAU | 428 | AUUUUCUC CUGAUGAG X CGAA AGCUUACU | 1564 |
| 1621 | GAAAAUGU U AUUUAGAA | 429 | UUCUAAAU CUGAUGAG X CGAA ACAUUUUC | 1565 |
| 1622 | AAAAUGUU A UUUAGAAA | 430 | UUUCUAAA CUGAUGAG X CGAA AACAUUUU | 1566 |
| 1624 | AAUGUUAU U UAGAAAAU | 431 | AUUUUCUA CUGAUGAG X CGAA AUAACAUU | 1567 |
| 1625 | AUGUUAUU U AGAAAAUC | 432 | GAUUUUCU CUGAUGAG X CGAA AAUAACAU | 1568 |
| 1626 | UGUUAUUU A GAAAAUCA | 433 | UGAUUUUC CUGAUGAG X CGAA AAAUAACA | 1569 |

Input Sequence = PLN. Cut Site = UH/.
Stem Length = 8. Core Sequence = CUGAUGAG X CGAA (X = GCCGUUAGGC or other stem II)
PLN (Homo sapiens phospholamban (PLN) mRNA.; 1635 bp)

TABLE IV
Human Phospholamban (PLN) NCH Ribozyme and Target Sequence

| Pos | Substrate | Seq ID | Ribozyme | Rz Seq ID |
|---|---|---|---|---|
| 15 | CAGAAAAC U AGCUAAAC | 434 | GUUUAGCU CUGAUGAG X CGAA IUUUUCUG | 1570 |
| 17 | GAAAACUC C CUAAACAC | 453 | GUGUUUAG CUGAUGAG X CGAA IAGUUUUC | 1571 |
| 18 | AAAACUCC C UAAACACC | 436 | GGUGUUUA CUGAUGAG X CGAA IGAGUUUU | 1572 |
| 19 | AAACUCCC C AAACACCC | 437 | GGGUGUUU CUGAUGAG X CGAA IGGAGUUU | 1573 |
| 20 | AACUCCCC A AACACCCG | 438 | CGGGUGUU CUGAUGAG X CGAA IGGGAGUU | 1574 |
| 23 | UCCCCAGC U ACCCGUAA | 439 | UUACGGGU CUGAUGAG X CGAA ICUGGGGA | 1575 |
| 28 | AGCUAAAC A UAAGACUU | 440 | AAGUCUUA CUGAUGAG X CGAA IUGUUUAG | 1576 |
| 30 | CUAAACAC C AGACUUCA | 441 | UGAAGUCU CUGAUGAG X CGAA IUGUUUAG | 1577 |
| 31 | UAAACACC C GACUUCAU | 442 | AUGAAGUC CUGAUGAG X CGAA IGUGUUUA | 1578 |
| 39 | CGUAAGAC U ACAACACA | 443 | UGUGUUGU CUGAUGAG X CGAA IUCUUACG | 1579 |
| 42 | AAGACUUC A ACACAAUA | 444 | UAUUGUGU CUGAUGAG X CGAA IAAGUCUU | 1580 |
| 46 | CUUCAUAC A AAUACUCU | 445 | AGAGUAUU CUGAUGAG X CGAA IUAUGAAG | 1581 |
| 49 | CAUACAAC A ACUCUAUA | 446 | UAUAGAGU CUGAUGAG X CGAA IUUGUAUG | 1582 |
| 51 | UACAACAC A UCUAUACU | 447 | AGUAUAGA CUGAUGAG X CGAA IUGUUGUA | 1583 |
| 56 | CACAAUAC U ACUGUGAU | 448 | AUCACAGU CUGAUGAG X CGAA IUAUUGUG | 1584 |
| 58 | CAAUACUC U UGUGAUGA | 449 | UCAUCACA CUGAUGAG X CGAA IAGUAUUG | 1585 |
| 63 | CUCUAUAC U UGAUCACA | 450 | UGUGAUCA CUGAUGAG X CGAA IUAUAGAG | 1586 |
| 73 | UGAUGAUC A UGCCAAGG | 451 | CCUUGGCA CUGAUGAG X CGAA IAUCAUCA | 1587 |
| 75 | AUGAUCAC A CCAAGGCU | 452 | AGCCUUGG CUGAUGAG X CGAA IUGAUCAU | 1588 |
| 78 | AUCACAGC U AGGCUACC | 453 | GGUAGCCU CUGAUGAG X CGAA ICUGUGAU | 1589 |
| 81 | ACAGCUGC C CUACCUAA | 454 | UUAGGUAG CUGAUGAG X CGAA ICAGCUGU | 1590 |
| 82 | CAGCUGCC A UACCUAAA | 455 | UUUAGGUA CUGAUGAG X CGAA IGCAGCUG | 1591 |
| 87 | GCCAAGGC U AAAAGAAG | 456 | CUUCUUJU CUGAUGAG X CGAA ICCIIGGC | 1592 |
| 90 | AAGGCUAC C AGAAGACA | 457 | UGUCUUCU CUGAUGAG X CGAA IUAGCCUU | 1593 |
| 91 | AGGCUACC U GAAGACAG | 458 | CUGUCUUC CUGAUGAG X CGAA IGUAGCCU | 1594 |
| 102 | AAGAAGAC A UCUCAUAU | 459 | AUAUGAGA CUGAUGAG X CGAA IUCUUCUU | 1595 |
| 109 | CAGUUAUC U UUUGGCUG | 460 | CAGCCAAA CUGAUGAG X CGAA IAUAACUG | 1596 |
| 111 | GUGAUCUC A UGGCUGCC | 461 | GGCAGCCA CUGAUGAG X CGAA IAGAUAAC | 1597 |
| 120 | UAUUUGGC U GCUUUUUU | 462 | UAAAAAGC CUGAUGAG X CGAA ICCAAAUA | 1598 |
| 123 | UUGGCUGC C UUUUAUCU | 463 | AGAUAAAA CUGAUGAG X CGAA ICAGCCAA | 1599 |
| 124 | UGGCUGCC A UUUAUCUU | 464 | AAGAUAAA CUGAUGAG X CGAA IGCAGCCA | 1600 |
| 127 | CUGCCAGC U AUCUUUCU | 465 | AGAAAGAU CUGAUGAG X CGAA ICUGGCAG | 1601 |
| 135 | UUUUUAUC U CUCGACCA | 466 | UGGUCGAG CUGAUGAG X CGAA IAUAAAAA | 1602 |
| 139 | UAUCUUUC U ACCACUUA | 467 | UAAGUGGU CUGAUGAG X CGAA IAAAGAUA | 1603 |
| 141 | UCUUUCUC U CACUUAAA | 468 | UUUAAGUG CUGAUGAG X CGAA IAGAAAGA | 1604 |
| 146 | CUCUCGAC C AAAACUUC | 469 | GAAGUUUU CUGAUGAG X CGAA IUCGAGAG | 1605 |
| 147 | UCUCGACC A AAACUUCA | 470 | UGAAGUUU CUGAUGAG X CGAA IGUCGAGA | 1606 |
| 149 | UCGACCAC U ACUUCAGA | 471 | UCUGAAGU CUGAUGAG X CGAA IUGGUCGA | 1607 |
| 156 | CUGAAAAC U ACUUCCUG | 472 | CAGGAAGU CUGAUGAG X CGAA IUUUUAAG | 1608 |
| 159 | AAAACUUC A UCCUGUCC | 473 | GGACAGGA CUGAUGAG X CGAA IAAGUUUU | 1609 |
| 163 | CUUCAGAC U GUCCUGCU | 474 | AGCAGGAC CUGAUGAG X CGAA IUCUGAAG | 1610 |
| 166 | CAGACUUC C CUGCUGGU | 475 | ACCAGOAG CUGAUGAG X CGAA IAAGUCUG | 1611 |
| 167 | AGACUUCC U UGCUGGUA | 476 | UACCAGCA CUGAUGAG X CGAA IGAAGUCU | 1612 |
| 171 | UUCCUGUC C GGUAUCAU | 477 | AUGAUACC CUGAUGAG X CGAA IACAGGAA | 1613 |
| 172 | UCCUGUCC U GUAUCAUG | 478 | CAUGAUAC CUGAUGAG X CGAA IGACAGGA | 1614 |
| 175 | UGUCCUOC U UCAUGGAG | 479 | CUCCAUGA CUGAUGAG X CGAA ICAGGACA | 1615 |
| 182 | CUGOVAUC A GAAAGUCC | 480 | GGACUUUC CUGAUGAG X CGAA IAUACCAG | 1616 |
| 194 | AGAAAGUC C CCUCACUC | 481 | GAGUGAGO CUGAUGAG X CGAA IACUUUCU | 1617 |
| 195 | GAAAGUCC A CUCACUCG | 482 | CGAGUGAG CUGAUGAG X CGAA IGACUUUC | 1618 |
| 200 | UCCAAUAC C UCGCUCAG | 483 | CUGAGCGA CUGAUGAG X CGAA IUAUUGGA | 1619 |
| 201 | CCAAUACC U CGCUCAGC | 484 | GCUGAGCG CUGAUGAG X CGAA IGUAUUGG | 1620 |
| 203 | AAUACCUC A CUCAGCUA | 485 | UAGCUGAG CUGAUGAG X CGAA IAGGUAUU | 1621 |
| 205 | UACCUCAC U CAGCUAUA | 486 | UAUAGCUG CUGAUGAG X CGAA IUGAGGUA | 1622 |
| 209 | UCACUCGC U UAUAAGAA | 487 | UUCUUAUA CUGAUGAG X CGAA ICGAGUGA | 1623 |
| 211 | ACUCGCUC A UAAGAAGA | 488 | UCUUCUUA CUGAUGAG X CGAA IAGCGAGU | 1624 |
| 214 | CGCUCAGC U GAAGAGCC | 489 | GGCUCUUC CUGAUGAG X CGAA ICUGAGCG | 1625 |
| 226 | AGAAGAGC C CCAUUGAA | 490 | UUCAAUGG CUGAUGAG X CGAA ICUCUUCU | 1626 |
| 227 | GAAGAGCC U CAUUGAAA | 491 | UUUCAAUG CUGAUGAG X CGAA IGCUCUUC | 1627 |
| 229 | AGAGCCUC A UUGAAAUG | 492 | CAUUUCAA CUGAUGAG X CGAA IAGGCUCU | 1628 |
| 232 | GCCUCAAC C AAAUGCCU | 493 | AGGCAUUU CUGAUGAG X CGAA IUUGAGGC | 1629 |
| 233 | CCUCAACC A AAUGCCUC | 494 | GAGGCAUU CUGAUGAG X CGAA IGUUGAGG | 1630 |
| 243 | UGAAAUGC C CAAGCACG | 495 | CGUGCUUG CUGAUGAG X CGAA ICAUUUCA | 1631 |
| 244 | GAAAUGCC U AAGCACGU | 499 | ACGUGCUU CUGAUGAG X CGAA IGCAUUUC | 1632 |
| 246 | AAUGCCUC A GCACGUCA | 497 | UGACGUGC CUGAUGAG X CGAA IAGGCAUU | 1633 |
| 249 | GCCUCAAC A CGUCAAAA | 499 | UUUUGACG CUGAUGAG X CUAA IUUGAGGC | 1634 |
| 253 | CAACAAGC A AAAAGCUA | 499 | UAGCUUUU CUGAUGAG X CGAA ICUUGUUG | 1635 |
| 258 | AGCACGUC A CUACAGAA | 502 | AUAAAUAG CUGAUGAG X CGAA IACGUGCU | 1636 |
| 264 | UCAAAAGC U AAUCUAUU | 521 | AAUAGAUU CUGAUGAG X CGAA ICUUUUGA | 1637 |
| 267 | AAAGCUAC A CUAUUUAU | 502 | AUAAAUAG CUGAUGAG X CGAA IUAGCUUU | 1638 |
| 273 | ACAGAAUC U AUCAAUUU | 503 | APAUUGAU CUGAUGAG X CGAA IAUUCUGU | 1639 |
| 281 | UAUUUAUC A CUGUCUCA | 504 | UGAGACAG CUGAUGAG X CGAA IAUAAAUA | 1640 |
| 287 | UCAAUUUC U CAUCUUAA | 505 | UUAAGAUF CUGAUGAG X CGAA IAAAUUGA | 1641 |
| 291 | UUUCUGUC U UUAAUAUG | 506 | CAUAUUAA CUGAUGAG X CGAA IACAGAAA | 1642 |
| 293 | UCUGUCUC A AAUAUGUC | 507 | GACAUAUU CUGAUGAG X CGAA IAGACAGA | 1643 |
| 296 | GUCUCAUC U AUGUCUCU | 508 | AGAGACAU CUGAUGAG X CGAA IAUGAGAC | 1644 |
| 306 | AAUAUGUC U CUGAUCUG | 509 | CAGAUCAG CUGAUGAG X CGAA IACAUAUU | 1645 |
| 308 | UAUGUCUC U GAUCUGUA | 510 | UACAGAUC CUGAUGAG X CGAA IAGACAUA | 1646 |
| 312 | UCUCUUGC U UGUAUCAU | 511 | AUGAUACA CUGAUGAG X CGAA ICAAGAGA | 1647 |
| 317 | UCCUGAUC U CAUCGUGA | 512 | UCACGAUG CUGAUGAG X CGAA IAUCAGCA | 1648 |
| 323 | UCUGUAUC A GAUGCUUC | 513 | GAAGCAUC CUGAUGAG X CGAA IAUACAGA | 1649 |
| 333 | CGUGAUGC U UGAAGUUC | 514 | GAACUUCA CUGAUGAG X CGAA ICAUCACG | 1650 |
| 336 | GAUGCUUC U AGUUCUGC | 515 | GCAGAACU CUGAUGAG X CGAA IAAGCAUC | 1652 |
| 338 | UGCUUCUC U UUCUGCUA | 516 | UAGCAGAA CUGAUGAG X CGAA IAGAAGCA | 1652 |
| 346 | UGAAGUUC U CAACCUCU | 517 | AGAGGUUG CUGAUGAG X CGAA IAACUUCA | 1653 |
| 349 | AGUUCUGC U CCUCUAGA | 518 | CUGAUGAG CUGAUGAG X CGAA ICAGAACU | 1654 |
| 352 | UCUGCUAC A CUAGAUCU | 519 | AGAUCUAG CUGAUGAG X CGAA IUAGCAGA | 1655 |
| 355 | GCUACAAC C GAUCUGCA | 520 | UGCAGAUC CUGAUGAG X CGAA IUUGUAGC | 1656 |
| 356 | CUACAACC U AUCUGCAG | 521 | CUGCAGAU CUGAUGAG X CGAA IGUUGUAG | 1657 |
| 358 | ACAACCUC U CUGCAGCU | 522 | AGCUGCAG CUGAUGAG X CGAA IAGGUUGU | 1658 |
| 364 | UCUAGAUC U CUUGCCAC | 523 | GUGGCAAG CUGAUGAG X CGAA IAUCUAGA | 1659 |
| 367 | AGAUCUGC A GCCACAUC | 524 | GAUGUGGC CUGAUGAG X CGAA ICAGAUCU | 1660 |
| 370 | UCUGCAGC U ACAUCAGC | 525 | GCUGAUGU CUGAUGAG X CGAA ICUGCAGA | 1661 |
| 374 | CAGCUUGC C CAGCUUAA | 526 | UUAAGCUG CUGAUGAG X CGAA ICAAGCUG | 1662 |
| 375 | AGCUUGCC A AGCUUAAA | 527 | UUUAAGCU CUGAUGAG X CGAA IGCAAGCU | 1663 |
| 377 | CUUGCCAC A CUUAAAAU | 528 | AUUUUAAG CUGAUGAG X CGAA IUGGCAAG | 1664 |
| 380 | GCCACAUC A AAAAUCUG | 529 | CAGAUUUU CUGAUGAG X CGAA IAUGUGGC | 1665 |
| 383 | ACAUCAGC U AUCUGUCA | 530 | UGACAGAU AUCUGUCA X CGAA ICUGAUGU | 1666 |
| 391 | UUAAAAUC U UCCCAUGC | 531 | GCAUGGGA CUGAUGAG X CGAA IAUUUUAA | 1667 |
| 395 | AAUCUGUC A AUGCAGAC | 532 | GUCUGCAU CUGAUGAG X CGAA IACAGAUU | 1668 |
| 398 | CUGUCAUC C CAGACAGG | 533 | CCUGUCUG CUGAUGAG X CGAA IAUGACAG | 1669 |
| 399 | UGUCAUCC C AGACAGGA | 534 | UCCUGUCU CUGAUGAG X CGAA IGAUGACA | 1670 |
| 400 | GUCAUCCC A GACAGGAA | 535 | UUCCUGUC CUGAUGAG X CGAA IGGAUGAC | 1671 |
| 404 | UCCCAUGC A GGAAAACA | 536 | UGUUUUCC CUGAUGAG X CGAA ICAUGGGA | 1672 |
| 408 | AUGCAGAC A AACAAUAU | 537 | AUAUUGUU CUGAUGAG X CGAA IUCUGCAU | 1673 |
| 416 | AGGAAAAC A UGUAUAAC | 538 | GUUAUACA CUGAUGAG X CGAA IUUUUCCU | 1674 |
| 429 | UGUAUAAC A ACUUCCUG | 539 | CAGGAAGU CUGAUGAG X CGAA IUUAUACA | 1675 |
| 433 | UAACAGAC C CCUGAGUA | 540 | UACUCAGG CUGAUGAG X CGAA IUCUGUUA | 1676 |
| 434 | AACAGACC A CUGAGUAG | 541 | CUACUCAG CUGAUGAG X CGAA IGUCUGUU | 1677 |
| 436 | CAGACCAC U GAGUAGAA | 542 | UUCUACUC CUGAUGAG X CGAA IUGGUCUG | 1678 |
| 439 | ACCACUUC C UAGAAGAG | 543 | CUCUUCUA CUGAUGAG X CGAA IAAGUGGU | 1679 |
| 440 | CCACUUCC U AGAAGAGU | 544 | ACUCUUCU CUGAUGAG X CGAA IGAAGUGG | 1680 |
| 456 | AGAGUUUC U GAAAAGGU | 545 | ACCUUUUC CUGAUGAG X CGAA IAAACUCU | 1681 |
| 470 | AAAAGGUC A UAAGACUA | 546 | UAGUCUUA CUGAUGAG X CGAA IACCUUUU | 1682 |
| 481 | AUUAAGAC U CUUAUUGU | 547 | ACAAUAAG CUGAUGAG X CGAA IUCUUAAU | 1683 |
| 487 | ACUAAAAC U CUUACCAU | 548 | AUGGUAAC CUGAUGAG X CGAA IUUUUAGU | 1684 |
| 497 | AUUGUUAC C GUAUUCAU | 549 | AUGAAUAC CUGAUGAG X CGAA IUAACAAU | 1685 |
| 498 | UUGUUACC A UAUUCAUC | 550 | GAUGAAUA CUGAUGAG X CGAA IGUAACAA | 1686 |
| 508 | AUGUAUUC A UUGGAUCU | 551 | AGAUCCAA CUGAUGAG X CGAA IAAUACAU | 1687 |
| 511 | UAUUCAUC U GAUCUUGU | 552 | ACAAGAUC CUGAUGAG X CGAA IAUGAAUA | 1688 |
| 520 | GUUGGAUC U AACAUGAA | 553 | UUCAUGUU CUGAUGAG X CGAA IAUCCAAC | 1689 |
| 528 | UUGUAAAC A AAGGGCUU | 554 | AAGCCCUU CUGAUGAG X CGAA IUUUACAA | 1690 |
| 539 | AAAAGGGC U UUUCAAAA | 555 | UUUUGAAA CUGAUGAG X CGAA ICCCUUUU | 1691 |
| 548 | UUAUUUUC A UUAACUUC | 556 | GAAGUUAA CUGAUGAG X CGAA IAAAAUAA | 1692 |
| 558 | AAAUUAAC U AAUAAGUG | 557 | CACUUAUU CUGAUGAG X CGAA IUUAAUUU | 1693 |
| 561 | UUAACUUC A AAGUGUAU | 558 | AUACACUU CUGAUGAG X CGAA IAAGUUAA | 1694 |
| 581 | UAAAAUGC A UUGAUUUC | 559 | GAAAUCAA CUGAUGAG X CGAA ICAUUUUA | 1695 |
| 584 | AAUGCAAC U AUUUCCUC | 560 | GAGGAAAU CUGAUGAG X CGAA IUUGCAUU | 1696 |
| 594 | UUGAUUUC C CAUGGCUC | 561 | GAGCCAUG CUGAUGAG X CGAA IAAAUCAA | 1697 |
| 595 | UGAUUUCC U AUGGCUCA | 562 | UGAGCCAU CUGAUGAG X CGAA IGAAAUCA | 1698 |
| 597 | AUUUCCUC A GGCUCACA | 563 | UGUGAGCC CUGAUGAG X CGAA IAGGAAAU | 1699 |
| 600 | UCCUCAAC A UCACAAAU | 564 | AUUUGUGA CUGAUGAG X CGAA IUUGAGGA | 1700 |
| 605 | AACAUGGC U AAUUUCUA | 565 | UAGAAAUU CUGAUGAG X CGAA ICCAUGUU | 1701 |
| 607 | CAUGGCUC A UUUCUAUC | 566 | GAUAGAAA CUGAUGAG X CGAA IAGCCAUG | 1702 |
| 609 | UGGCUCAC A UCUAUCCC | 567 | GGGAUAGA CUGAUGAG X CGAA IUGAGCCA | 1703 |
| 616 | CAAAUUUC U CAAAUCUU | 568 | AAGAUUUG CUGAUGAG X CGAA IAAAUUUG | 1704 |
| 620 | UUUCUAUC C UCUUUUCU | 569 | AGAAAAGA CUGAUGAG X CGAA IAUAGAAA | 1705 |
| 621 | UUCUAUCC C CUUUUCUG | 570 | CAGAAAAG CUGAUGAG X CGAA IGAUAGAA | 1706 |
| 622 | UCUAUCCC A UUUUCUGA | 571 | UCAGAAAA CUGAUGAG X CGAA IGGAUAGA | 1707 |
| 627 | CCCAAAUC U UGAAGAUG | 572 | CAUCUUCA CUGAUGAG X CGAA IAUUUGGG | 1708 |
| 632 | AUCUUUUC U AUGAAGAG | 573 | CUCUUCAU CUGAUGAG X CGAA IAAAAGAU | 1709 |
| 659 | UUUAAAAC U UGCCAACA | 574 | UGUUGGCA CUGAUGAG X CGAA IUUUUAAA | 1710 |
| 662 | AAAACUGC A CAACAAGU | 575 | ACUUGUUG CUGAUGAG X CGAA ICAGUUUU | 1711 |
| 664 | AACUGCAC U ACAAGUUC | 576 | GAACUUGU CUGAUGAG X CGAA IUGCAGUU | 1712 |
| 667 | UGCACUGC C AGUUCACU | 577 | AGUGAACU CUGAUGAG X CGAA ICAGUGCA | 1713 |
| 668 | GCACUGCC A GUUCACUU | 578 | AAGUGAAC CUGAUGAG X CGAA IGCAGUGC | 1714 |
| 671 | CUGCCAAC A CACUUCAU | 579 | AUGAAGUG CUGAUGAG X CGAA IUUGGCAG | 1715 |
| 677 | ACAAGUUC A AUAUAUAA | 580 | UUAUAUAU CUGAUGAG X CGAA IAACUUGU | 1716 |
| 679 | AAGUUCAC U AUAUAAAG | 581 | CUUUAUAU CUGAUGAG X CGAA IUGAACUU | 1717 |
| 682 | UUCACUUC A UAAAGCAU | 582 | AUGCUUUA CUGAUGAG X CGAA IAAGUGAA | 1718 |
| 693 | UAUAAAGC A UUUUACUC | 583 | GAGUAAAA CUGAUGAG X CGAA ICUUUAUA | 1719 |
| 704 | AUUUUUAC U UGAGGUGA | 584 | UCACCUCA CUGAUGAG X CGAA IUAAAAAU | 1720 |
| 706 | UUUUACUC U AGGUGAAU | 585 | AUUCACCU CUGAUGAG X CGAA IAGUAAAA | 1721 |
| 733 | UAUAUUAC A AAAAGCUU | 586 | AAGCUUUU CUGAUGAG X CGAA IUAAUAUA | 1722 |
| 744 | GUAAAAGC U UAAUACUA | 587 | UAGUAUUA CUGAUGAG X CGAA ICUUUUAC | 1723 |
| 747 | AAAGCUUC U UACUAAGU | 588 | ACUUAGUA CUGAUGAG X CGAA IAAGCUUU | 1724 |
| 755 | UUUAAUAC U AUUUUUCA | 589 | AUUUUUCA CUGAUGAG X CGAA IUAUUAAA | 1725 |
| 767 | UAUUUUUC A UUCACCAA | 590 | UUGGUGAA CUGAUGAG X CGAA IAAAAAUA | 1726 |
| 772 | UUCAGGUC U CAAGUAUC | 591 | GAUACUUG CUGAUGAG X CGAA IACCUGAA | 1727 |
| 775 | AGGUCUUC A GUAUCAAA | 592 | UUUGAUAC CUGAUGAG X CGAA IAAGACCU | 1728 |
| 777 | GUCUUCAC C AUCAAAGU | 593 | ACUUUGAU CUGAUGAG X CGAA IUGAAGAC | 1729 |
| 778 | UCUUCACC A UCAAAGUA | 594 | UACUUUGA CUGAUGAG X CGAA IGUGAAGA | 1730 |
| 785 | CAAGUAUC A AAUAACAC | 595 | GUGUUAUU CUGAUGAG X CGAA IAUACUUG | 1731 |
| 796 | GUAAUAAC A UGAAGUGU | 596 | ACACUUCA CUGAUGAG X CGAA IUUAUUAC | 1732 |
| 798 | AAUAACAC A AAGUGUCA | 597 | UGACACUU CUGAUGAG X CGAA IUGUUAUU | 1733 |
| 810 | GAAGUGUC A UCAAAAUA | 598 | UAUUUUGA CUGAUGAG X CGAA IACACUUC | 1734 |
| 817 | CAUUAUUC A AGUCCACU | 599 | AGUGGACU CUGAUGAG X CGAA IAAUAAUG | 1735 |
| 826 | AAAUAGUC C ACUCCUCA | 600 | UGAGGAGU CUGAUGAG X CGAA IACUAUUU | 1736 |
| 827 | AAUAGUCC A CUCCUCAC | 601 | GUGAGGAG CUGAUGAG X CGAA IGACUAUU | 1737 |
| 829 | UAGUCCAC U CCUCACAU | 602 | AUGUGAGG CUGAUGAG X CGAA IUGGACUA | 1738 |
| 833 | CCACUGAC U ACAUCUGU | 603 | ACAGAUGU CUGAUGAG X CGAA IUCAGUGG | 1739 |
| 835 | ACUGACUC C AUCUGUUA | 604 | UAACAGAU CUGAUGAG X CGAA IAGUCAGU | 1740 |
| 836 | CUGACUCC U UCUGUUAU | 605 | AUAACAGA CUGAUGAG X CGAA IGAGUCAG | 1741 |
| 838 | GACUCCUC A UGUUAUCU | 606 | AGAUAACA CUGAUGAG X CGAA IAGGAGUC | 1742 |
| 840 | CUCCUCAC A UUAUCUUA | 607 | UAAGAUAA CUGAUGAG X CGAA IUGAGGAG | 1743 |
| 843 | CUCACAUC U UCUUAUUA | 608 | UAAUAAGA CUGAUGAG X CGAA IAUGUGAG | 1744 |
| 850 | CUGUUAUC U AUAAAGAA | 609 | UUCUUUAU CUGAUGAG X CGAA IAUAACAG | 1745 |
| 864 | UAAAGAAC U GUAGUAAC | 610 | GUUACUAC CUGAUGAG X CGAA IUUCUUUA | 1746 |
| 877 | GUAGUAAC U GAAUCUAC | 611 | GUAGAUUC CUGAUGAG X CGAA IAUAGUUA | 1747 |
| 881 | UAACUAUC A CUACAUUC | 612 | GAAUGUAG CUGAUGAG X CGAA IAUAGUUA | 1748 |
| 887 | UCAGAAUC U UCUAAAAC | 613 | GUUUUAGA CUGAUGAG X CGAA IAUUCUGA | 1749 |
| 890 | GAAUCUAC A AAAACAGA | 614 | UCUGUUUU CUGAUGAG X CGAA IUAGAUUC | 1750 |
| 894 | CUACAUUC U CAGAAAUU | 615 | AAUUUCUG CUGAUGAG X CGAA IAAUGUAG | 1751 |
| 900 | UCUAAAAC A UUGUAUUU | 616 | AAAUACAA CUGAUGAG X CGAA IUUUUAGA | 1752 |
| 917 | AUUUUUUC U CACAUUAA | 617 | UUAAUGUG CUGAUGAG X CGAA IAAAAAAU | 1753 |
| 922 | UUCUAUGC C UAACAUCU | 618 | AGAUGUUA CUGAUGAG X CGAA ICAUAGPA | 1754 |
| 923 | UCUAUGCC A AACAUCUU | 619 | AAGAUGUU CUGAUGAG X CGAA IGCAUAGA | 1755 |
| 925 | UAUGCCAC A CAUCUUUU | 620 | AAAAGAUG CUGAUGAG X CGAA IUGGCAUA | 1756 |
| 931 | ACAUUAAC A UUAAAGUU | 621 | AACUUUAA CUGAUGAG X CGAA IUUAAUGU | 1757 |
| 934 | UUAACAUC U AAGUUGAU | 622 | AUCAACUU CUGAUGAG X CGAA IAUGUUAA | 1758 |
| 954 | UGAGAAUC A UGGAAAAG | 623 | CUUUUCCA CUGAUGAG X CGAA IAUUCUCA | 1759 |
| 973 | AGUAAGGC C UCUUACAU | 624 | AUGUAAGA CUGAUGAG X CGAA ICCUUACU | 1760 |
| 974 | GUAAGGCC A CUUACAUA | 625 | UAUGUAAG CUGAUGAG X CGAA IGCCUUAC | 1761 |
| 978 | GGCCAUAC U CAUAAUAA | 626 | UUAUUAUG CUGAUGAG X CGAA IUAUGGCC | 1762 |
| 980 | CCAUACUC U UAAUAAAA | 627 | UUUUAUUA CUGAUGAG X CGAA IAGUAUGG | 1763 |
| 984 | ACUCUUAC A AAAAUUCC | 628 | GGAAUUUU CUGAUGAG X CGAA IUAAGAGU | 1764 |
| 996 | UAAAAUUC C AAGUAAUU | 629 | AAUUACUU CUGAUGAG X CGAA IAAUUUUA | 1765 |
| 997 | AAAAUUCC U AGUAAUUU | 630 | AAAUUACU CUGAUGAG X CGAA IGAAUUUU | 1766 |
| 1014 | AUUUUUUC A AUCACAGA | 631 | UCUGUGAU CUGAUGAG X CGAA IAAAAAAU | 1767 |
| 1022 | AAAGAAUC A AUUCUAGU | 632 | ACUAGAAU CUGAUGAG X CGAA IAUUCUUU | 1768 |
| 1024 | AGAAUCAC A UCUAGUAC | 633 | GUACUAGA CUGAUGAG X CGAA IUGAUUCU | 1769 |
| 1031 | CAGAAUUC U CAUGUAGG | 634 | CCUACAUG CUGAUGAG X CGAA IAAUUCUG | 1770 |
| 1037 | UCUAGUAC A GGUAAAUC | 635 | GAUUUACC CUGAUGAG X CGAA IUACUAGA | 1771 |
| 1050 | GGUAAAUC A UCUGUUCU | 636 | AGAACAGA CUGAUGAG X CGAA IAUUUACC | 1772 |
| 1057 | CAUAAAUC U UAAGACAU | 637 | AUGUCUUA CUGAUGAG X CGAA IAUUUAUG | 1773 |
| 1062 | AUCUGUUC U CAUAUGAU | 638 | AUCAUAUG CUGAUGAG X CGAA IAACAGAU | 1774 |
| 1068 | UCUAAGAC A AUCAACAG | 639 | CUGUUGAU CUGAUGAG X CGAA IUCUUAGA | 1775 |
| 1076 | AUAUGAUC A AUGAGAAC | 640 | GUUCUCAU CUGAUGAG X CGAA IAUCAUAU | 1776 |
| 1079 | UGAUCAAC A AGAACUGG | 641 | CCAGUUCU CUGAUGAG X CGAA IUUGAUCA | 1777 |
| 1089 | AUGAGAAC U GUUAAUAU | 642 | AUAUUAAC CUGAUGAG X CGAA IUUCUCAU | 1778 |
| 1107 | UAUGUGAC A GAUUAGUC | 643 | GACUAAUC CUGAUGAG X CGAA IUCACAUA | 1779 |
| 1120 | GAUUAGUC A ACUAAUAU | 644 | AUAUUAGU CUGAUGAG X CGAA IACUAAUC | 1780 |
| 1125 | GUCAUAUC A UAUACUAA | 645 | UUAGUAUA CUGAUGAG X CGAA IAUAUGAC | 1781 |
| 1127 | CAUAUCAC U UACUAACA | 646 | UGUUAGUA CUGAUGAG X CGAA IUGAUAUG | 1782 |
| 1135 | UAAUAUAC U ACAGAAUC | 647 | GAUUCUGU CUGAUGAG X CGAA IUAUAUUA | 1783 |
| 1139 | AUACUAAC A AAUCUAAU | 648 | AUUAGAUU CUGAUGAG X CGAA IUUAGUAU | 1784 |
| 1142 | CUAACAAC A CUAAUCUU | 649 | AAGAUUAG CUGAUGAG X CGAA IUUGUUAG | 1785 |
| 1148 | ACAGAAUC U UUCAUUUA | 650 | UAAAUGAA CUGAUGAG X CGAA IAUUCUGU | 1786 |
| 1153 | AUCUAAUC U UUAAGGCA | 651 | UGCCUUAA CUGAUGAG X CGAA IAUUAGAU | 1787 |
| 1156 | UAAUCUUC A AGGCACUG | 652 | CAGUGCCU CUGAUGAG X CGAA IAAGAUUA | 1788 |
| 1165 | UUUAAGGC A AGUGAAUU | 653 | AAUUCACU CUGAUGAG X CGAA ICCUUAAA | 1789 |
| 1167 | UAAGGCAC U UGAAUUAU | 654 | AUAAUUCA CUGAUGAG X CGAA IUGCCUUA | 1790 |
| 1181 | GAAUUAUC U UAGAGUUA | 655 | UAACUCUA CUGAUGAG X CGAA IAUAAUUC | 1791 |
| 1186 | AUCUGAGC U UUACCUAG | 656 | CUAGGUAA CUGAUGAG X CGAA ICUCAGAU | 1792 |
| 1195 | AGAGUUAC C UUACCAUA | 657 | UAUGGUAA CUGAUGAG X CGAA IUAACUCU | 1793 |
| 1196 | GAGUUACC U UACCAUAC | 658 | GUAUGGUA CUGAUGAG X CGAA IGUAACUC | 1794 |
| 1200 | UACCUAGC U AUACUAUA | 659 | UAUAGUAU CUGAUGAG X CGAA ICUAGGUA | 1795 |
| 1204 | UAGCUUAC C UAUAUCUU | 660 | AAGAUAUA CUGAUGAG X CGAA IUAAGCUA | 1796 |
| 1205 | AGCUUACC A AUAUCUUU | 661 | AAAGAUAU CUGAUGAG X CGAA IGUAAGCU | 1797 |
| 1209 | UACCAUAC U CUUUGGAA | 662 | UUCCAAAG CUGAUGAG X CGAA IUAUGGUA | 1798 |
| 1215 | ACUAUAUC U AAUCAUGA | 663 | UCAUGAUU CUGAUGAG X CGAA IAUAUAGU | 1799 |
| 1224 | UUGGAAUC A ACCUUAAG | 664 | CUUAAGGU CUGAUGAG X CGAA IAUUCCAA | 1800 |
| 1231 | CAUGAAAC C GACUUCAG | 665 | CUGAAGUC CUGAUGAG X CGAA IUUUCAUG | 1801 |
| 1232 | AUGAAACC U ACUUCAGA | 666 | UCUGAAGU CUGAUGAG X CGAA IGUUUCAU | 1802 |
| 1239 | CUUAAGAC U AAUGAUUU | 667 | AAAUCAUU CUGAUGAG X CGAA IUCUUAAG | 1803 |
| 1242 | AAGACUUC A GAUUUUGC | 668 | GCAAAAUC CUGAUGAG X CGAA IAAGUCUU | 1804 |
| 1255 | GAUUUUGC A GUCUUCCA | 669 | UGGAAGAC CUGAUGAG X CGAA ICAAAAUC | 1805 |
| 1263 | AGGUUGUC U UUCCAGCC | 670 | GGCUGGAA CUGAUGAG X CGAA IACAACCU | 1806 |
| 1266 | UUGUCUUC C CAGCCUAA | 671 | UUAGGCUG CUGAUGAG X CGAA IAAGACAA | 1807 |
| 1267 | UGUCUUCC A AGCCUAAC | 672 | GUUAGGCU CUGAUGAG X CGAA IGAAGACA | 1808 |
| 1271 | UUCCAUUC C UAACAUCC | 673 | GGAUGUUA CUGAUGAG X CGAA IAAUGGAA | 1809 |
| 1272 | UCCAUUCC A AACAUCCA | 674 | UGGAUGUU CUGAUGAG X CGAA IGAAUGGA | 1810 |
| 1275 | AUUCCAGC C AUCCAAUG | 675 | CAUUGGAU CUGAUGAG X CGAA ICUGGAAU | 1811 |
| 1276 | UUCCAGCC U UCCAAUGC | 676 | GCAUUGGA CUGAUGAG X CGAA IGCUGGAA | 1812 |
| 1280 | AGCCUAAC A AUGCAGGC | 677 | GCCUGCAU CUGAUGAG X CGAA IUUAGGCU | 1813 |
| 1283 | CUAACAUC C CAGGCAAG | 678 | CUUGCCUG CUGAUGAG X CGAA IUUAGGCU | 1814 |
| 1284 | UAACAUCC A AGGCAAGG | 679 | CCUUGCCU CUGAUGAG X CGAA IGAUGUUA | 1815 |
| 1289 | UCCAAUGC A AGGAAAAU | 680 | AUUUUCCU CUGAUGAG X CGAA ICAUUGGA | 1816 |
| 1293 | AUGCAGGC A AAAUAAAA | 681 | UUUUAUUU CUGAUGAG X CGAA ICCUGCAU | 1817 |
| 1312 | AAGAUUUC C ACAGAAAA | 682 | UUUUCUGU CUGAUGAG X CGAA IAAAUCUU | 1818 |
| 1313 | AGAUUUCC A CAGAAAAA | 683 | UUUUUCUG CUGAUGAG X CGAA IGAAAUCU | 1819 |
| 1319 | CCAGUGAC A AAUAUAUU | 684 | AAUAUAUU CUGAUGAG X CGAA IUCACUGG | 1820 |
| 1335 | AUAUUAUC U UAUUUUUU | 685 | AAAAAAUA CUGAUGAG X CGAA IAUAAUAU | 1821 |
| 1337 | AUUAUCUC A UUUUUUAA | 686 | UUAAAAAA CUGAUGAG X CGAA IAGAUAAU | 1822 |
| 1364 | AUGAAUUC U CCAAAUAU | 687 | AUAUUUGG CUGAUGAG X CGAA IAAUUCAU | 1823 |
| 1366 | GAAUUCUC U AAAUAUUA | 688 | UAAUAUUU CUGAUGAG X CGAA IAGAAUUC | 1824 |
| 1368 | AUUCUCUC U AUAUUAAC | 689 | GUUAAUAU CUGAUGAG X CGAA IAGAGAAU | 1825 |
| 1370 | UCUCUCUC C AUUAACUA | 690 | UAGUUAUU CUGAUGAG X CGAA IAGAGAGA | 1826 |
| 1371 | CUCUCUCC A UUAACUAA | 691 | UUAGUUAA CUGAUGAG X CGAA IGAGAGAG | 1827 |
| 1381 | AUAUUAAC U AUUAGAUU | 692 | AAUCUAAU CUGAUGAG X CGAA IUUAAUAU | 1828 |
| 1410 | AAAUGAAC U GGCCCAUC | 693 | GAUGGGCC CUGAUGAG X CGAA IUUCAUUU | 1829 |
| 1418 | UUGUUGGC C UAUUACAU | 694 | AUGUAAUA CUGAUGAG X CGAA ICCAACAA | 1830 |
| 1419 | UGUUGGCC C AUUACAUC | 695 | GAUGUAAU CUGAUGAG X CGAA IGCCAACA | 1831 |
| 1420 | GUUGGCCC A UUACAUCU | 696 | AGAUGUAA CUGAUGAG X CGAA IGGCCAAC | 1832 |
| 1423 | GGCCCAUC U CAUCUACA | 697 | UGUAGAUG CUGAUGAG X CGAA IAUGGGCC | 1833 |
| 1429 | UCUAUUAC A CAGCUGAC | 698 | GUCAGOUG CUGAUGAG X CGAA IUAAUAGA | 1834 |
| 1432 | AUUACAUC U CUGACCCU | 699 | AGGGUCAG CUGAUGAG X CGAA IAUGUAAU | 1835 |
| 1435 | ACAUCUAC A ACCCUUGA | 700 | UCAAGGGU CUGAUGAG X CGAA IUAGAUGU | 1836 |
| 1438 | UCUACAGC U CUUGAACA | 701 | UGUUCAAG CUGAUGAG X CGAA ICUGUAGA | 1837 |
| 1442 | CAGCUGAC C AACAUGGG | 702 | CCCAUGUU CUGAUGAG X CGAA IUCAGCUG | 1838 |
| 1443 | AGCUGACC C ACAUGGGG | 703 | CCCCAUGU CUGAUGAG X CGAA IGUCAGCU | 1839 |
| 1444 | GCUGACCC U CAUGGGGG | 704 | CCCCCAUG CUGAUGAG X CGAA IGGUCAGC | 1840 |
| 1450 | CCUUGAAC A GGUUAGGG | 705 | CCCUAACC CUGAUGAG X CGAA IUUCAAGG | 1841 |
| 1467 | AGGGGAGC U AUUCGUGG | 706 | CCACGAAU CUGAUGAG X CGAA ICUCCCCU | 1842 |
| 1471 | GAGCUGAC A GUGGGUCC | 707 | GGACCCAC CUGAUGAG X CGAA IUCAGCUC | 1843 |
| 1483 | CGUGGGUC C AAUCUUAA | 708 | UUAAGAUU CUGAUGAG X CGAA IACCCACG | 1844 |
| 1486 | GGGUCCGC A CUUAACUA | 709 | UAGUUAAG CUGAUGAG X CGAA ICGGACCC | 1845 |
| 1492 | GCAAAAUC U UACCUAAU | 710 | AUUAGGUA CUGAUGAG X CGAA IAUUUUGC | 1846 |
| 1497 | AUCUUAAC U AAUAGCCU | 711 | AGGCUAUU CUGAUGAG X CGAA IUUAAGAU | 1847 |
| 1500 | UUAACUAC C AGCCUACU | 712 | AGUAGGCU CUGAUGAG X CGAA IUAGUUAA | 1848 |
| 1501 | UAACUACC U GCCUACUA | 713 | UAGUAGGC CUGAUGAG X CGAA IGUAGUUA | 1849 |
| 1508 | CUAAUAGC C AUUGACCA | 714 | UGGUCAAU CUGAUGAG X CGAA ICUAUUAG | 1850 |
| 1509 | UAAUAGCC U UUGACCAU | 715 | AUGGUCAA CUGAUGAG X CGAA IGCUAUUA | 1851 |
| 1512 | UAGCCUAC U ACCAUAAA | 716 | UUUAUGGU CUGAUGAG X CGAA IUCAAUAG | 1852 |
| 1519 | CUAUUGAC C ACCUUACU | 717 | AGUAAGGU CUGAUGAG X CGAA IUCAAUAG | 1853 |
| 1520 | UAUUGACC A CCUUACUG | 718 | CAGUAAGG CUGAUGAG X CGAA IGUCAAUA | 1854 |
| 1526 | CCAUAAAC C UGAUAACA | 719 | UGUUAUCA CUGAUGAG X CGAA IUUUAUGG | 1855 |
| 1527 | CAUAAACC U GAUAACAU | 720 | AUGUUAUC CUGAUGAG X CGAA IGUUUAUG | 1856 |
| 1531 | AACCUUAC U ACAUAAAC | 721 | GUUUAUGU CUGAUGAG X CGAA IUAAGGUU | 1857 |
| 1538 | CUGAUAAC A CAGUAAAU | 722 | AUUUACUG CUGAUGAG X CGAA IUUAUCAG | 1858 |
| 1544 | ACAUAAAC A AUUAACAC | 723 | GUGUUAAU CUGAUGAG X CGAA IUUUAUGU | 1859 |
| 1555 | AAAUUAAC A UUUUGCGU | 724 | ACGCAAAA CUGAUGAG X CGAA IUUAAUUU | 1860 |
| 1557 | AUUAACAC A UUGCGUGU | 725 | ACACGCAA CUGAUGAG X CGAA IUGUUAAU | 1861 |
| 1584 | UAUUAUAC A AUUCCUAC | 726 | GUAGGAAU CUGAUGAG X CGAA IUAUAAUA | 1862 |
| 1586 | UUAUACAC U UCCUACAA | 727 | UUGUAGGA CUGAUGAG X CGAA IUGUAUAA | 1863 |
| 1593 | CUAUAUUC C AUAAAGUA | 728 | UACUUUAU CUGAUGAG X CGAA IAAUAUAG | 1864 |
| 1594 | UAUAUUCC U UAAAGUAA | 729 | UUACUUUA CUGAUGAG X CGAA IGAAUAUA | 1865 |
| 1597 | AUUCCUAC A AGUAAGCU | 730 | AGCUUACU CUGAUGAG X CGAA IUAGGAAU | 1866 |
| 1609 | AAGUAAGC U AAAAUGUU | 731 | AACAUUUU CUGAUGAG X CGAA ICUUACUU | 1867 |

Input Sequence = PLN. Cut Site = CH/.
Stem Length = 8. Core Sequence = CUGAUGAG X CGAA (X = GCCGUUAGGC or other stem II)
PLN (Homo sapiens phospholamban (PLN) mRNA.; 1635 bp)

TABLE V
Human Phospholamban (PLN) G-cleaver Ribozyme and Target Sequence

| Pos | Substrate | Seq ID | Ribozyme | Rz Seq ID |
|---|---|---|---|---|
| 64 | UCUAUACU G UGAUGAUC | 732 | GAUCAUCA UGAUG GCAUGCACUAUGC GCG AGUAUAGA | 1868 |
| 66 | UAUACUGU G AUGAUCAC | 733 | GUGAUCAU UGAUG GCAUGCACUAUGC GCG ACAGUAUA | 1869 |
| 69 | ACUGUGAU G AUCACAGC | 734 | GCUGUGAU UGAUG GCAUGCACUAUGC GCG AUCACAGU | 1870 |
| 79 | UCACAGCU G CCAAGGCU | 735 | AGCCUUGG UGAUG GCAUGCACUAUGC GCG AGCUGUGA | 1871 |
| 121 | AUUUGGCU G CCAGCUUU | 736 | AAAGCUGG UGAUG GCAUGCACUAUGC GCG AGCCAAAU | 1872 |
| 143 | UUUCUCUC G ACCACUUA | 737 | UAAGUGGU UGAUG GCAUGCACUAUGC GCG GAGAGAAA | 1873 |
| 168 | GACUUCCU G UCCUGCUG | 738 | CAGCAGGA UGAUG GCAUGCACUAUGC GCG AGGAAGUC | 1874 |
| 173 | CCUGUCCU G CUGGUAUC | 739 | GAUACCAG UGAUG GCAUGCACUAUGC GCG AGGACAGG | 1875 |
| 207 | CCUCACUC G CUCAGCUA | 740 | UAGCUGAG UGAUG GCAUGCACUAUGC GCG GAGUGAGG | 1876 |
| 236 | CAACCAUU G AAAUGCCU | 741 | AGGCAUUU UGAUG GCAUGCACUAUGC GCG AAUGGUUG | 1877 |
| 241 | AUUGAAAU G CCUCAACA | 742 | UGUUGAGG UGAUG GCAUGCACUAUGC GCG AUUUCAAU | 1878 |
| 288 | CAAUUUCU G UCUCAUCU | 743 | AGAUGAGA UGAUG GCAUGCACUAUGC GCG AGAAAUUG | 1879 |
| 303 | CUUAAUAU G UCUCUUGC | 744 | GCAAGAGA UGAUG GCAUGCACUAUGC GCG AUAUUAAG | 1880 |
| 310 | UGUCUCUU G CUGAUCUG | 745 | CAGAUCAG UGAUG GCAUGCACUAUGC GCG AAGAGACA | 1881 |
| 313 | CUCUUGCU G AUCUGUAU | 746 | AUACAGAU UGAUG GCAUGCACUAUGC GCG AGCAAGAG | 1882 |
| 318 | GCUGAUCU G UAUCAUCG | 747 | CGAUGAUA UGAUG GCAUGCACUAUGC GCG AGAUCAGC | 1883 |
| 328 | AUCAUCGU G AUGCUUCU | 748 | AGAAGCAU UGAUG GCAUGCACUAUGC GCG ACGAUGAU | 1884 |
| 331 | AUCGUGAU G CUUCUCUG | 749 | CAGAGAAG UGAUG GCAUGCACUAUGC GCG AUCACGAU | 1885 |
| 339 | GCUUCUCU G AAGUUCUG | 750 | CAGAACUU UGAUG GCAUGCACUAUGC GCG AGAGAAGC | 1886 |
| 347 | GAAGUUCU G CUACAACC | 751 | GGUUGUAG UGAUG GCAUGCACUAUGC GCG AGAACUUC | 1887 |
| 365 | CUAGAUCU G CAGCUUGC | 752 | GCAAGCUG UGAUG GCAUGCACUAUGC GCG AGAUCUAG | 1888 |
| 372 | UGCAGCUU G CCACAUCA | 753 | UGAUGUGG UGAUG GCAUGCACUAUGC GCG AAGCUGCA | 1889 |
| 392 | UAAAAUCU G UCAUCCCA | 754 | UGGGAUGA UGAUG GCAUGCACUAUGC GCG AUGGGAUG | 1890 |
| 402 | CAUCCCAU G CAGACAGG | 755 | CCUGUCUG UGAUG GCAUGCACUAUGC GCG AUGGGAUG | 1891 |
| 422 | ACAAUAUU G UAUAACAG | 756 | CUGUUAUA UGAUG GCAUGCACUAUGC GCG AAUAUUGU | 1892 |
| 441 | CACUUCCU G AGUAGAAG | 757 | CUUCUACU UGAUG GCAUGCACUAUGC GCG AGGAAGUG | 1893 |
| 459 | GUUUCUUU U UGAAAAGG | 758 | CCUUUUCA UGAUG GCAUGCACUAUGC GCG AAAGAAAC | 1894 |
| 461 | UUCUUUGU G AAAAGGUC | 759 | GACCUUUU UGAUG GCAUGCACUAUGC GCG ACAAAGAA | 1895 |
| 492 | AACUUAUU U UUACCAUA | 760 | UAUGGUAA UGAUG GCAUGCACUAUGC GCG AAUAAGUU | 1896 |
| 502 | UACCAUAU G UAUUCAUC | 761 | GAUGAAUA UGAUG GCAUGCACUAUGC GCG AUAUGGUA | 1897 |
| 512 | AUUCAUCU G UUGGAUCU | 762 | AGAUCCAA UGAUG GCAUGCACUAUGC GCG AGAUGAAU | 1898 |
| 522 | UGGAUCUU G UAAACAUG | 763 | CAUGUUUA UGAUG GCAUGCACUAUGC GCG AAGAUCCA | 1899 |
| 530 | GUAAACAU G AAAAGGGC | 764 | GCCCUUUU UGAUG GCAUGCACUAUGC GCG AUGUUUAC | 1900 |
| 570 | AAAUAAGU U UAUAAAAU | 765 | AUUUUAUA UGAUG GCAUGCACUAUGC GCG ACUUAUUU | 1901 |
| 579 | UAUAAAAU G CAACUGUU | 766 | AACAGUUG UGAUG GCAUGCACUAUGC GCG AUUUUAUA | 1902 |
| 585 | AUGCAACU G UUGAUUUC | 767 | GAAAUCAA UGAUG GCAUGCACUAUGC GCG AGUUGCAU | 1903 |
| 586 | CAACUGUU G AUUUCCUC | 768 | GAGGAAAU UGAUG GCAUGCACUAUGC GCG AACAGUUG | 1904 |
| 633 | UCUUUUCU G AAGAUGAA | 769 | UUCAUCUU UGAUG GCAUGCACUAUGC GCG AGAAAAGA | 1905 |
| 639 | CUGAAGAU G AAGAGUUU | 770 | AAACUCUU UGAUG GCAUGCACUAUGC GCG AUCUUCAG | 1906 |
| 660 | UUAAAACU G CACUGCCA | 771 | UGGCAGUG UGAUG GCAUGCACUAUGC GCG AGUUUUAA | 1907 |
| 665 | ACUGCACU G CCAACAAG | 772 | CUUGUUGG UGAUG GCAUGCACUAUGC GCG AGUGCAGU | 1908 |
| 710 | ACUCUUUU G AGGUGAAU | 773 | AUUCACCU UGAUG GCAUGCACUAUGC GCG AAAAGAGU | 1909 |
| 715 | UUUGAGGU G AAUAUAAU | 774 | AUUAUAUU UGAUG GCAUGCACUAUGC GCG ACCUCAAA | 1910 |
| 736 | AUUACAAU G UAAAAGCU | 775 | AGCUUUUA UGAUG GCAUGCACUAUGC GCG AUUGUAAU | 1911 |
| 802 | ACACAAAU G AAGUGUCA | 776 | UGACACUU UGAUG GCAUGCACUAUGC GCG AUUUGUGU | 1912 |
| 807 | AAUGAAGU G UCAUUAUU | 777 | AAUAAUGA UGAUG GCAUGCACUAUGC GCG ACUUCAUU | 1913 |
| 830 | AGUCCACU G ACUCCUCA | 778 | UGAGGAGU UGAUG GCAUGCACUAUGC GCG AGUGGACU | 1914 |
| 844 | UCACAUCU G UUAUCUUA | 779 | UAAGAUAA UGAUG GCAUGCACUAUGC GCG AGAUGUGA | 1915 |
| 869 | AACUAUUU G UAGUAACU | 780 | AGUUACUA UGAUG GCAUGCACUAUGC GCG AAAUAGUU | 1916 |
| 907 | CAGAAAUU G UAUUUUUU | 781 | AAAAAAUA UGAUG GCAUGCACUAUGC GCG AAUUUCUG | 1917 |
| 920 | UUUUCUAU G CCACAUUA | 782 | UAAUGUGG UGAUG GCAUGCACUAUGC GCG AUAGAAAA | 1918 |
| 944 | UUAAAGUU G AUGAGAAU | 783 | AUUCUCAU UGAUG GCAUGCACUAUGC GCG AACUUUAA | 1919 |
| 947 | AAGUUGAU G AGAAUCAA | 784 | UUGAUUCU UGAUG GCAUGCACUAUGC GCG AUCAACUU | 1920 |
| 1039 | UAGUACAU G UAGGUAAA | 785 | UUUACCUA UGAUG GCAUGCACUAUGC GCG AUGUACUA | 1921 |
| 1058 | AUAAAUCU G UUCUAAGA | 786 | UCUUAGAA UGAUG GCAUGCACUAUGC GCG AGAUUUAU | 1922 |
| 1072 | AGACAUAU G AUCAACAG | 787 | CUGUUGAU UGAUG GCAUGCACUAUGC GCG AUAUGUCU | 1923 |
| 1083 | CAACAGAU G AGAACUGG | 788 | CCAGUUCU UGAUG GCAUGCACUAUGC GCG AUCUGUUG | 1924 |
| 1102 | GUUAAUAU G UGACAGUG | 789 | CACUGUCA UGAUG GCAUGCACUAUGC GCG AUAUUAAC | 1925 |
| 1104 | UAAUAUGU G ACAGUGAG | 790 | CUCACUGU UGAUG GCAUGCACUAUGC GCG ACAUAUUA | 1926 |
| 1110 | GUGACAGU G AGAUUAGU | 791 | ACUAAUCU UGAUG GCAUGCACUAUGC GCG ACUGUCAC | 1927 |
| 1168 | AAGGCACU G UAGUGAAU | 792 | AUUCACUA UGAUG GCAUGCACUAUGC GCG AGUGCCUU | 1928 |
| 1173 | ACUGUAGU G AAUUAUCU | 793 | AGAUAAUU UGAUG GCAUGCACUAUGC GCG ACUACAGU | 1929 |
| 1182 | AAUUAUCU G AGCUAGAG | 794 | CUCUAGCU UGAUG GCAUGCACUAUGC GCG AGAUAAUU | 1930 |
| 1226 | GGAAUCAU G AAACCUUA | 795 | UAAGGUUU UGAUG GCAUGCACUAUGC GCG AUGAUUCC | 1931 |
| 1247 | UUCAGAAU G AUUUUGCA | 796 | UGCAAAAU UGAUG GCAUGCACUAUGC GCG AUUCUGAA | 1932 |
| 1253 | AUGAUUUU G CAGGUUGU | 797 | ACAACCUG UGAUG GCAUGCACUAUGC GCG AAAAUCAU | 1933 |
| 1260 | UGCAGGUU G UCUUCCAU | 798 | AUGGAAGA UGAUG GCAUGCACUAUGC GCG AACCUGCA | 1934 |
| 1287 | CAUCCAAU G CAGGCAAG | 799 | CUUGCCUG UGAUG GCAUGCACUAUGC GCG AUUGGAUG | 1935 |
| 1316 | UUUCCAGU G ACAGAAAA | 800 | UUUUCUGU UGAUG GCAUGCACUAUGC GCG ACUGGAAA | 1936 |
| 1358 | AAAUAUAU G AAUUCUCU | 801 | AGAGAAUU UGAUG GCAUGCACUAUGC GCG AUAUAUUU | 1937 |
| 1401 | UAUAUUUU G AAAUGAAC | 802 | GUUCAUUU UGAUG GCAUGCACUAUGC GCG AAAAUAUA | 1938 |
| 1406 | UUUGAAAU G AACUUGUU | 803 | AACAAGUU UGAUG GCAUGCACUAUGC GCG AUUUCAAA | 1939 |
| 1412 | AUGAACUU G UUGGCCCA | 804 | UGGGCCAA UGAUG GCAUGCACUAUGC GCG AAGUUCAU | 1940 |
| 1439 | CUACAGCU G ACCCUUGA | 805 | UCAAGGGU UGAUG GCAUGCACUAUGC GCG AGCUGUAG | 1941 |
| 1446 | UGACCCUU G AACAUGGG | 806 | CCCAUGUU UGAUG GCAUGOACUAUGC GCG AAGGGUCA | 1942 |
| 1468 | GGGGAGCU G ACAAUUCG | 807 | CGAAUUGU UGAUG GCAUGCACUAUGC GCG AGCUCCCC | 1943 |
| 1484 | GUGGGUCC G CAAAAUCU | 808 | AGAUUUUG UGAUG GCAUGCACUAUGC GCG GGACCCAC | 1944 |
| 1516 | CUACUAUU G ACCAUAAA | 809 | UUUAUGGU UGAUG GCAUGCACUAUGC GCG AAUAGUAG | 1945 |
| 1532 | ACCUUACU G AUAACAUA | 810 | UAUGUUAU UGAUG GCAUGCACUAUGC GCG AGUAAGGU | 1946 |
| 1564 | CAUAUUUU G CGUGUUAU | 811 | AUAACACG UGAUG GCAUGCACUAUGC GCG AAAAUAUG | 1947 |
| 1568 | UUUUGCGU G UUAUAUGU | 812 | ACAUAUAA UGAUG GCAUGCACUAUGC GCG ACGCAAAA | 1948 |
| 1575 | UGUUAUAU G UAUUAUAC | 813 | GUAUAAUA UGAUG GCAUGCACUAUGC GCG AUAUAACA | 1949 |
| 1619 | GAGAAAAU G UUAUUUAG | 814 | CUAAAUAA UGAUG GCAUGCACUAUGC GCG AUUUUCUC | 1950 |

Input Sequence = PLN. Cut Site = YG/M or UG/U.
Stem Length = 8. Core Sequence = UGAUG GCAUGCACUAUGC GCG
PLN (Homo sapiens phospholamban (PLN) mRNA.; 1635 bp)

TABLE VI
Human Phospholamban (PLN) zinzyme Ribozyme and Target Sequence

| Pos | Substrate | Seq ID | Ribozyme | Rz Seq ID |
|---|---|---|---|---|
| 64 | UCUAUACU G UGAUGAUC | 732 | GAUCAUCA GCCGAAAGGCGAGUCAAGGUCU AGUAUAGA | 1951 |
| 79 | UCACAGCU G CCAAGGCU | 735 | AGCCUUGG GCCGAAAGGCGAGUCAAGGUCU AGCUGUGA | 1952 |
| 121 | AUUUGGCU G CCAGCUUU | 736 | AAAGCUGG GCCGAAAGGCGAGUCAAGGUCU AGCCAAAU | 1953 |
| 168 | GACUUCCU G UCCUGCUG | 738 | CAGCAGGA GCCGAAAGGCGAGUCAAGGUCU AGGAAGUC | 1954 |
| 173 | CCUGUCCU G CUGGUAUC | 739 | GAUACCAG GCCGAAAGGCGAGUCAAGGUCU AGGACAGG | 1955 |
| 207 | CCUCACUC G CUCAGCUA | 740 | UAGCUGAG GCCGAAAGGCGAGUCAAGGUCU GAGUGAGG | 1956 |
| 241 | AUUGAAAU G CCUCAACA | 742 | UGUUGAGG GCCGAAAGGCGAGUCAAGGUCU AUUUCAAU | 1957 |
| 288 | CAAUUUCU G UCUCAUCU | 743 | AGAUGAGA GCCGAAAGGCGAGUCAAGGUCU AGAAAUUG | 1958 |
| 303 | CUUAAUAU G UCUCUUGC | 744 | GCAAGAGA GCCGAAAGGCGAGUCAAGGUCU AUAUUAAG | 1959 |
| 310 | UGUCUCUU G CUGAUCUG | 745 | CAGAUCAG GCCGAAAGGCGAGUCAAGGUCU AAGAGACA | 1960 |
| 318 | GCUGAUCU G UAUCAUCG | 747 | CGAUGAUA GCCGAAAGGCGAGUCAAGGUCU AGAUCAGC | 1961 |
| 331 | AUCGUGAU G CUUCUCUG | 749 | CAGAGAAG GCCGAAAGGCGAGUCAAGGUCU AUCACGAU | 1962 |
| 347 | GAAGUUCU G CUACAACC | 751 | GGUUGUAG GCCGAAAGGCGAGUCAAGGUCU AGAACUUC | 1963 |
| 365 | CUAGAUCU G CAGCUUGC | 752 | GCAAGCUG GCCGAAAGGCGAGUCAAGGUCU AGAUCUAG | 1964 |
| 372 | UGCAGCUU G CCACAUCA | 753 | UGAUGUGG GCCGAAAGGCGAGUCAAGGUCU AAGCUGCA | 1965 |
| 392 | UAAAAUCU G UCAUCCCA | 754 | UGGGAUGA GCCGAAAGGCGAGUCAAGGUCU AGAUUUUA | 1966 |
| 402 | CAUCCCAU G CAGACAGG | 755 | CCUGCCUG GCCGAAAGGCGAGUCAAGGUCU AUGGGAUG | 1967 |
| 422 | ACAAUAUU G UAUAACAG | 756 | CUGUUAUA GCCGAAAGGCGAGUCAAGGUCU AAUAUUGU | 1968 |
| 459 | GUUUCUUU G UGAAAAGG | 758 | CCUUUUCA GCCGAAAGGCGAGUCAAGGUCU AAAGAAAC | 1969 |
| 492 | AACUUAUU G UUACCAUA | 760 | UAUGGUAA GCCGAAAGGCGAGUCAAGGUCU AAUAAGUU | 1970 |
| 502 | UACCAUAU G UAUTCAUC | 761 | GAUGAAUA GCCGAAAGGCGAGUCAAGGUCU AUAUGGUA | 1971 |
| 512 | AUUCAUCU G UUGGAUCU | 762 | AGAUCCAA GCCGAAAGGCGAGUCAAGGUCU AGAUGAAU | 1972 |
| 522 | UGGAUCUU G UAAACAUG | 763 | CAUGUUUA GCCGAAAGGCGAGUCAAGGUCU AAGAUCCA | 1973 |
| 570 | AAAUAAGU G UAUAAAAU | 765 | AUUUUAUA GCCGAAAGGCGAGUCAAGGUCU ACUUAUUU | 1974 |
| 579 | UAUAAAAU G CAACUGUU | 766 | AACAGUUG GCCGAAAGGCGAGUCAAGGUCU AUUUUAUA | 1975 |
| 585 | AUGCAACU G UUGAUUUC | 767 | GAAAUCAA GCCGAAAGGCGAGUCAAGGUCU AGUUGCAU | 1976 |
| 660 | UUAAAACU G CACUGCCA | 771 | UGGCAGUG GCCGAAAGGCGAGUCAAGGUCU AGUUUUAA | 1977 |
| 665 | ACUGCACU G CCAACAAG | 772 | CUUGUUGG GCCGAAAGGCGAGUCAAGGUCU AGUGCAGU | 1976 |
| 736 | AUUACAAU G UAAAAGCU | 775 | AGCUUUUA GCCGAAAGGCGAGUCAAGGUCU AUUGUAAU | 1979 |
| 807 | AAUGAAGU G UCAUUAUU | 777 | AAUAAUGA GCCGAAAGGCGAGUCAAGGUCU ACUUCAUU | 1980 |
| 844 | UCACAUCU G UUAUCUUA | 779 | UAAGAUAA GCCGAAAGGCGAGUCAAGGUCU AGAUGUGA | 1951 |
| 869 | AACUAUUU G UAGUAACU | 780 | AGUUACUA GCCGAAAGGCGAGUCAAGGUCU AAAUAGUU | 1982 |
| 907 | CAGAAAUU G UAUUUUUU | 781 | AAAAAAUA GCCGAAAGGCGAGUCAAGGUCU AAUUUCUG | 1983 |
| 920 | UUUUCUAU G CCACAUUA | 782 | UAAUGUGG GCCGAAAGGCGAGUCAAGGUCU AUAGAAAA | 1984 |
| 1039 | UAGUACAU G UAGGUAAA | 785 | UUUACCUA GCCGAAAGGCGAGUCAAGGUCU AUGUACUA | 1985 |
| 1058 | AUAAAUCU G UUCUAAGA | 786 | UCUUAGAA GCCGAAAGGCGAGUCAAGGUCU AGAUUUAU | 1986 |
| 1102 | GUUAAUAU G UGACAGUG | 789 | CACUGUCA GCCGAAAGGCGAGUCAAGGUCU AUAUUAAC | 1987 |
| 1168 | AAGGCACU G UAGUGAAU | 792 | AUUCACUA GCCGAAAGGCGAGUCAAGGUCU AGUGCCUU | 1988 |
| 1253 | AUGAUUUU G CAGGUUGU | 797 | ACAACCUG GCCGAAAGGCGAGUCAAGGUCU AAAAUCAU | 1989 |
| 1260 | UGCAGGUU G UCUUCCAU | 798 | AUGGAAGA GCCGAAAGGCGAGUCAAGGUCU AACCUGCA | 1990 |
| 1287 | CAUCCAAU G CAGGCAAG | 799 | CUUGCCUG GCCGAAAGGCGAGUCAAGGUCU AUUGGAUG | 1992 |
| 1412 | AUGAACUU G UUGGCCCA | 804 | UGGGCCAA GCCGAAAGGCGAGUCAAGGUCU AAGUUCAU | 1992 |
| 1484 | GUGGGUCC G CAAAAUCU | 808 | AGAUUUUG GCCGAAAGGCGAGUCAAGGUCU GGACCCAC | 1993 |
| 1564 | CAUAUUUU G CGUGUUAU | 811 | AUAACACG GCCGAAAGGCGAGUCAAGGUCU AAAAUAUG | 1994 |
| 1568 | UUUUGCGU G UUAUAUGU | 812 | ACAUAUAA GCCGAAAGGCGAGUCAAGGUCU ACGCAAAA | 1995 |
| 1575 | UGUUAUAU G UAUUAUAC | 813 | GUAUAAUA GCCGAAAGGCGAGUCAAGGUCU AUAUAACA | 1996 |
| 1619 | GAGAAAAU G UUAUUUAG | 814 | CUAAAUAA GCCGAAAGGCGAGUCAAGGUCU AUUUUCUC | 1997 |
| 21 | ACUCCCCA G CUAAACAC | 815 | GUGUUUAG GCCGAAAGGCGAGUCAAGGUCU UGGGGAGU | 1998 |
| 32 | AAACACCC G UAAGACUU | 816 | AAGUCUUA GCCGAAAGGCGAGUCAAGGUCU GGGUGUUU | 1999 |
| 76 | UGAUCACA G CUGCCAAG | 817 | CUUGGCAG GCCGAAAGGCGAGUCAAGGUCU UGUGAUCA | 2000 |
| 85 | CUGCCAAG G CUACCUAA | 818 | UUAGGUAG GCCGAAAGGCGAGUCAAGGUCU CUUGGCAG | 2001 |
| 103 | AGAAGACA G UUAUCUCA | 819 | UGAGAUAA GCCGAAAGGCGAGUCAAGGUCU UGUCUUCU | 2002 |
| 118 | CAUAUUUG G CUGCCAGC | 820 | GCUGGCAG GCCGAAAGGCGAGUCAAGGUCU CAAAUAUG | 2003 |
| 125 | GGCUGCCA G CUUUUUAU | 821 | AUAAAAAG GCCGAAAGGCGAGUCAAGGUCU UGGCAGCC | 2004 |
| 177 | UCCUGCUG G UAUCAUGG | 822 | CCAUGAUA GCCGAAAGGCGAGUCAAGGUCU CAGCAGGA | 2005 |
| 191 | UGGAGAAA G UCCAAUAC | 823 | GUAUUGGA GCCGAAAGGCGAGUCAAGGUCU UUUCUCCA | 2006 |
| 212 | CUCGCUCA G CUAUAAGA | 824 | UCUUAUAG GCCGAAAGGCGAGUCAAGGUCU UGAGCGAG | 2007 |
| 224 | UAAGAAGA G CCUCAACC | 825 | GGUUGAGG GCCGAAAGGCGAGUCAAGGUCU UCUUCUUA | 2008 |
| 251 | CUCAACAA G CACGUCAA | 826 | UUGACGUG GCCGAAAGGCGAGUCAAGGUCU UUGUUGAG | 2009 |
| 255 | ACAAGCAC G UCAAAAGC | 827 | GCUUUUGA GCCGAAAGGCGAGUCAAGGUCU GUGCUUGU | 2010 |
| 262 | CGUCAAAA G CUACAGAA | 828 | UUCUGUAG GCCGAAAGGCGAGUCAAGGUCU UUUUGACG | 2011 |
| 326 | GUAUCAUC G UGAUGCUU | 829 | AAGCAUCA GCCGAAAGGCGAGUCAAGGUCU GAUGAUAC | 2012 |
| 342 | UCUCUGAA G UUCUGCUA | 830 | UAGCAGAA GCCGAAAGGCGAGUCAAGGUCU UUCAGAGA | 2013 |
| 368 | GAUCUGCA G CUUGCCAC | 831 | GUGGCAAG GCCGAAAGGCGAGUCAAGGUCU UGCAGAUC | 2014 |
| 381 | CCACAUCA G CUUAAAAU | 832 | AUUUUAAG GCCGAAAGGCGAGUCAAGGUCU UGAUGUGG | 2015 |
| 443 | CUUCCUGA G UAGAAGAG | 833 | CUCUUCUA GCCGAAAGGCGAGUCAAGGUCU UCAGGAAG | 2016 |
| 451 | GUAGAAGA G UUUCUUUG | 834 | CAAAGAAA GCCGAAAGGCGAGUCAAGGUCU UCUUCUAC | 2017 |
| 467 | GUGAAAAG G UCAAGAUU | 835 | AAUCUUGA GCCGAAAGGCGAGUCAAGGUCU CUUUUCAC | 2018 |
| 537 | UGAAAAGG G CUUUAUUU | 836 | AAAUAAAG GCCGAAAGGCGAGUCAAGGUCU CCUUUUCA | 2019 |
| 568 | CAAAAUAA G UGUAUAAA | 837 | UUUAUACA GCCGAAAGGCGAGUCAAGGUCU UUAUUUUG | 2020 |
| 603 | UCAACAUG G CUCACAAA | 838 | UUUGUGAG GCCGAAAGGCGAGUCAAGGUCU CAUGUUGA | 2021 |
| 644 | GAUGAAGA G UUUAGUUU | 839 | AAACUAAA GCCGAAAGGCGAGUCAAGGUCU UCUUCAUC | 2022 |
| 649 | AGAGUUUA G UUUUAAAA | 840 | UUUUAAAA GCCGAAAGGCGAGUCAAGGUCU UAAACUCU | 2023 |
| 673 | GCCAACAA G UUCACUUC | 841 | GAAGUGAA GCCGAAAGGCGAGUCAAGGUCU UUGUUGGC | 2024 |
| 691 | UAUAUAAA G CAUUAUUU | 842 | AAAUAAUG GCCGAAAGGCGAGUCAAGGUCU UUUAUAUA | 2025 |
| 713 | CUUUUGAG G UGAAUAUA | 843 | UAUAUUCA GCCGAAAGGCGAGUCAAGGUCU CUCAAAAG | 2026 |
| 742 | AUGUAAAA G CUUCUUUA | 844 | UAAAGAAG GCCGAAAGGCGAGUCAAGGUCU UUUUACAU | 2027 |
| 758 | AAUACUAA G UAUUUUUC | 845 | GAAAAAUA GCCGAAAGGCGAGUCAAGGUCU UUAGUAUU | 2028 |
| 769 | UUUUUCAG G UCUUCACC | 846 | GGUGAAGA GCCGAAAGGCGAGUCAAGGUCU CUGAAAAA | 2029 |
| 780 | UUCACCAA G UAUCAAAG | 847 | CUUUGAUA GCCGAAAGGCGAGUCAAGGUCU UUGGUGAA | 2030 |
| 788 | GUAUCAAA G UAAUAACA | 848 | UGUUAUUA GCCGAAAGGCGAGUCAAGGUCU UUUGAUAC | 2031 |
| 805 | CAAAUGAA G UGUCAUUA | 849 | UAAUGACA GCCGAAAGGCGAGUCAAGGUCU UUCAUUUG | 2032 |
| 823 | UCAAAAUA G UCCACUGA | 850 | UCAGUGGA GCCGAAAGGCGAGUCAAGGUCU UAUUUUGA | 2033 |
| 872 | UAUUUGUA G UAACUAUC | 851 | GAUAGUUA GCCGAAAGGCGAGUCAAGGUCU UACAAAUA | 2034 |
| 941 | CUUUUAAA G UUGAUGAG | 852 | CUCAUCAA GCCGAAAGGCGAGUCAAGGUCU UUUAAAAG | 2035 |
| 956 | AGAAUCAA G UAUGGAAA | 853 | UUUCCAUA GCCGAAAGGCGAGUCAAGGUCU UUGAUUCU | 2036 |
| 966 | AUGGAAAA G UAAGGCCA | 854 | UGGCCUUA GCCGAAAGGCGAGUCAAGGUCU UUUUCCAU | 2037 |
| 971 | AAAGUAAG G CCAUACUC | 855 | GAGUAUGG GCCGAAAGGCGAGUCAAGGUCU CUUACUUU | 2038 |
| 1003 | CCUUUUAA G UAAUUUUU | 856 | AAAAAUUA GCCGAAAGGCGAGUCAAGGUCU UUAAAAGG | 2039 |
| 1033 | GAAUUCUA G UACAUGUA | 857 | UACAUGUA GCCGAAAGGCGAGUCAAGGUCU UAGAAUUC | 2040 |
| 1043 | ACAUGUAG G UAAAUCAU | 858 | AUGAUUUA GCCGAAAGGCGAGUCAAGGUCU CUACAUGU | 2041 |
| 1091 | GAGAACUG G UGGUUAAU | 859 | AUUAACCA GCCGAAAGGCGAGUCAAGGUCU CAGUUCUC | 2042 |
| 1094 | AACUGGUG G UUAAUAUG | 860 | CAUAUUAA GCCGAAAGGCGAGUCAAGGUCU CACCAGUU | 2043 |
| 1108 | AUGUGACA G UGAGAUUA | 861 | UAAUCUCA GCCGAAAGGCGAGUCAAGGUCU UGUCACAU | 2044 |
| 1117 | UGAGAUUA G UCAUAUCA | 862 | UGAUAUGA GCCGAAAGGCGAGUCAAGGUCU UAAUCUCA | 2045 |
| 1163 | CAUUUAAG G CACUGUAG | 863 | CUACAGUG GCCGAAAGGCGAGUCAAGGUCU CUUAAAUG | 2046 |
| 1171 | GCACUGUA G UGAAUUAU | 864 | AUAAUUCA GCCGAAAGGCGAGUCAAGGUCU UACAGUGC | 2047 |
| 1184 | UUAUCUGA G CUAGAGUU | 865 | AACUCUAG GCCGAAAGGCGAGUCAAGGUCU UCAGAUAA | 2048 |
| 1190 | GAGCUAGA G UUACCUAG | 866 | CUAGGUAA GCCGAAAGGCGAGUCAAGGUCU UCUAGCUC | 2049 |
| 1198 | GUUACCUA G CUUACCAU | 867 | AUGGUAAG GCCGAAAGGCGAGUCAAGGUCU UAGGUAAC | 2050 |
| 1257 | UUUUGCAG G UUGUCUUC | 868 | GAAGACAA GCCGAAAGGCGAGUCAAGGUCU CUGCAAAA | 2051 |
| 1273 | CCAUUCCA G CCUAACAU | 869 | AUGUUAGG GCCGAAAGGCGAGUCAAGGUCU UGGAAUGG | 2052 |
| 1291 | CAAUGCAG G CAAGGAAA | 870 | UUUCCUUG GCCGAAAGGCGAGUCAAGGUCU CUGCAUUG | 2053 |
| 1314 | GAUUUCCA G UGACAGAA | 871 | UUCUGUCA GCCGAAAGGCGAGUCAAGGUCU UGGAAAUC | 2054 |
| 1339 | UAUCUCAA G UAUUUUUU | 872 | AAAAAAUA GCCGAAAGGCGAGUCAAGGUCU UUGAGAUA | 2055 |
| 1416 | ACUUGUUG G CCCAUCUA | 873 | UAGAUGGG GCCGAAAGGCGAGUCAAGGUCU CAACAAGU | 2056 |
| 1436 | CAUCUACA G CUGACCCU | 874 | AGGGUCAG GCCGAAAGGCGAGUCAAGGUCU UGUAGAUG | 2057 |
| 1456 | ACAUGGGG G UUAGGGGA | 875 | UCCCCUAA GCCGAAAGGCGAGUCAAGGUCU CCCCAUGU | 2058 |
| 1465 | UUAGGGGA G CUGACAAU | 876 | AUUGUCAG GCCGAAAGGCGAGUCAAGGUCU UCCCCUAA | 2059 |
| 1476 | GACAAUUC G UGGGUCCG | 877 | CGGACCCA GCCGAAAGGCGAGUCAAGGUCU GAAUUGUC | 2060 |
| 1480 | AUUCGUGG G UCCGCAAA | 878 | UUUGCGGA GCCGAAAGGCGAGUCAAGGUCU CCACGAAU | 2061 |
| 1506 | ACCUAAUA G CCUACUAU | 879 | AUAGUAGG GCCGAAAGGCGAGUCAAGGUCU UAUUAGGU | 2062 |
| 1545 | CAUAAACA G UAAAUUAA | 880 | UUAAUUUA GCCGAAAGGCGAGUCAAGGUCU UGUUUAUG | 2063 |
| 1566 | UAUUUUGC G UGUUAUAU | 881 | AUAUAACA GCCGAAAGGCGAGUCAAGGUCU GCAAAAUA | 2064 |
| 1603 | ACAAUAAA G UAAGCUAG | 882 | CUAGCUUA GCCGAAAGGCGAGUCAAGGUCU UUUAUUGU | 2065 |
| 1607 | UAAAGUAA G CUAGAGAA | 883 | UUCUCUAG GCCGAAAGGCGAGUCAAGGUCU UUACUUUA | 2066 |

Input Sequence = PLN. Cut Site = G/Y
Stem Length = 8. Core Sequence = GCcgaaagGCGaGuCaaGGuCu
PLN (Homo sapiens phospholamban (PLN) mRNA.; 1635 bp)

TABLE VII
Human Phospholamban (PLN) DNAzyme and Target Sequence

| Pos | Substrate | Seq ID | Ribozyme | Rz Seq ID |
|---|---|---|---|---|
| 44 | GACUUCAU A CAACACAA | 6 | TTGTGTTG GGCTAGCTACAACGA ATGAAGTC | 2067 |
| 54 | AACACAAU A CUCUAUAC | 7 | GTATAGAG GGCTAGCTACAACGA ATTGTGTT | 2068 |
| 59 | AAUACUCU A UACUGUGA | 9 | TCACAGTA GGCTAGCTACAACGA AGAGTATT | 2069 |
| 61 | UACUCUAU A CUGUGAUG | 10 | CATCACAG GGCTAGCTACAACGA ATAGAGTA | 2070 |
| 88 | CCAAGGCU A CCUAAAAG | 12 | CTTTTAGG GGCTAGCTACAACGA AGCCTTGG | 2071 |
| 106 | AGACAGUU A UCUCAUAU | 15 | ATATGAGA GGCTAGCTACAACGA AACTGTCT | 2072 |
| 113 | UAUCUCAU A UUUGGCUG | 18 | CAGCCAAA GGCTAGCTACAACGA ATGAGATA | 2073 |
| 132 | AGCUUUUU A UCUUUCUC | 25 | GAGAAAGA GGCTAGCTACAACGA AAAAAGCT | 2074 |
| 179 | CUGCUGGU A UCAUGGAG | 39 | CTCCATGA GGCTAGCTACAACGA ACCAGCAG | 2075 |
| 198 | AGUCCAAU A CCUCACUC | 42 | GAGTGAGG GGCTAGCTACAACGA ATTGGACT | 2076 |
| 215 | GCUCAGCU A UAAGAAGA | 46 | TCTTCTTA GGCTAGCTACAACGA AGCTGAGC | 2077 |
| 265 | CAAAAGCU A CAGAAUCU | 52 | AGATTCTG GGCTAGCTACAACGA AGCTTTTG | 2078 |
| 274 | CAGAAUCU A UUUAUCAA | 54 | TTGATAAA GGCTAGCTACAACGA AGATTCTG | 2079 |
| 278 | AUCUAUUU A UCAAUUUC | 57 | GAAATTGA GGCTAGCTACAACGA AAATAGAT | 2080 |
| 301 | AUCUUAAU A UGUCUCUU | 67 | AAGAGACA GGCTAGCTACAACGA ATTAAGAT | 2081 |
| 320 | UGAUCUGU A UCAUCGUG | 72 | CACGATGA GGCTAGCTACAACGA ACAGATCA | 2082 |
| 350 | GUUCUGCU A CAACCUCU | 80 | AGAGGTTG GGCTAGCTACAACGA AGCAGAAC | 2083 |
| 419 | AAAACAAU A UUGUAUAA | 91 | TTATACAA GGCTAGCTACAACGA ATTGTTTT | 2084 |
| 424 | AAUAUUGU A UAACAGAC | 93 | GTCTGTTA GGCTAGCTACAACGA ACAATATT | 2085 |
| 489 | UAAAACUU A UUGUUACC | 108 | GGTAACAA GGCTAGCTACAACGA AAGTTTTA | 2086 |
| 495 | UUAUUGUU A CCAUAUGU | 111 | ACATATGG GGCTAGCTACAACGA AACAATAA | 2087 |
| 500 | GUUACCAU A UGUAYYCA | 112 | TGAATACA GGCTAGCTACAACGA ATGGTAAC | 2088 |
| 504 | CCAUAUGU A UUCAUCUG | 113 | CAGATGAA GGCTAGCTACAACGA ACATATGG | 2089 |
| 542 | AGGGCUUU A UUUUCAAA | 123 | TTTGAAAA GGCTAGCTACAACGA AAAGCCCT | 2090 |
| 572 | AUAAGUGU A UAAAAUGC | 133 | GCATTTTA GGCTAGCTACAACGA ACACTTAT | 2091 |
| 617 | AAAUUUCU A UCCCAAAU | 144 | ATTTGGGA GGCTAGCTACAACGA AGAAATTT | 2092 |
| 684 | CACUUCAU A UAUAAAGC | 162 | GCTTTATA GGCTAGCTACAACGA ATGAAGTG | 2093 |
| 686 | CUUCAUAU A UAAAGCAU | 163 | ATGCTTTA GGCTAGCTACAACGA ATATGAAG | 2094 |
| 696 | AAAGCAUU A UUUUUACU | 166 | AGTAAAAA GGCTAGCTACAACGA AATGCTTT | 2095 |
| 702 | UUAUUUUU A CUCUUUUG | 171 | CAAAAGAG GGCTAGCTACAACGA AAAAATAA | 2096 |
| 719 | AGGUGAAU A UAAUUUAU | 176 | ATAAATTA GGCTAGCTACAACGA ATTCACCT | 2097 |
| 726 | UAUAAUUU A UAUUACAA | 180 | TTGTAATA GGCTAGCTACAACGA AAATTATA | 2098 |
| 728 | UAAUUUAU A UUACAAUG | 181 | CATTGTAA GGCTAGCTACAACGA ATAAATTA | 2099 |
| 731 | UUUAUAUU A CAAUGUAA | 183 | TTACATTG GGCTAGCTACAACGA AATATAAA | 2100 |
| 753 | UCUUUAAU A CUAAGUAU | 190 | ATACTTAG GGCTAGCTACAACGA ATTAAAGA | 2101 |
| 760 | UACUAAGU A UUUUUCAG | 192 | CTGAAAAA GGCTAGCTACAACGA ACTTAGTA | 2102 |
| 782 | CACCAAGU A UCAAAGUA | 201 | TACTTTGA GGCTAGCTACAACGA ACTTGGTG | 2103 |
| 813 | GUGUCAUU A UUCAAAAU | 207 | ATTTTGAA GGCTAGCTACAACGA AATGACAC | 2104 |
| 847 | CAUCUGUU A UCUUAUUA | 216 | TAATAAGA GGCTAGCTACAACGA AACAGATG | 2105 |
| 852 | GUUAUCUU A UUAUAAAG | 219 | CTTTATAA GGCTAGCTACAACGA AAGATAAC | 2106 |
| 855 | AUCUUAUU A UAAAGAAC | 221 | GTTCTTTA GGCTAGCTACAACGA AATAAGAT | 2107 |
| 865 | AAAGAACU A UUUGUAGU | 223 | ACTACAAA GGCTAGCTACAACGA AGTTCTTT | 2108 |
| 878 | UAGUAACU A UCAGAAUC | 228 | GATTCTGA GGCTAGCTACAACGA AGTTACTA | 2109 |
| 888 | CAGAAUCU A CAUUCUAA | 231 | TTAGAATG GGCTAGCTACAACGA AGATTCTG | 2110 |
| 909 | GAAAUUGU A UUUUUUCU | 236 | AGAAAAAA GGCTAGCTACAACGA ACAATTTC | 2111 |
| 918 | UUUUUUCU A UGCCACAU | 243 | ATGTGGCA GGCTAGCTACAACGA AGAAAAAA | 2112 |
| 958 | AAUCAAGU A UGGAAAAG | 253 | CTTTTCCA GGCTAGCTACAACGA ACTTGATT | 2113 |
| 976 | AAGGCCAU A CUCUUACA | 255 | TGTAAGAG GGCTAGCTACAACGA ATGGCCTT | 2114 |
| 982 | AUACUCUU A CAUAAUAA | 258 | TTATTATG GGCTAGCTACAACGA AAGAGTAT | 2115 |
| 1035 | AUUCUAGU A CAUGUAGG | 278 | CCTACATG GGCTAGCTACAACGA ACTAGAAT | 2116 |
| 1070 | UAAGACAU A UGAUCAAC | 287 | GTTGATCA GGCTAGCTACAACGA ATGTCTTA | 2117 |
| 1100 | UGGUUAAU A UGUGACAG | 291 | CTGTCACA GGCTAGCTACAACGA ATTAACCA | 2118 |
| 1122 | UUAGUCAU A UCACUAAU | 295 | ATTAGTGA GGCTAGCTACAACGA ATGACTAA | 2119 |
| 1131 | UCACUAAU A UACUAACA | 298 | TGTTAGTA GGCTAGCTACAACGA ATTAGTGA | 2120 |
| 1133 | ACUAAUAU A CUAACAAC | 299 | GTTGTTAG GGCTAGCTACAACGA ATATTAGT | 2121 |
| 1178 | AGUGAAUU A UCUGAGCU | 311 | AGCTCAGA GGCTAGCTACAACGA AATTCACT | 2122 |
| 1193 | CUAGAGUU A CCUAGCUU | 315 | AAGCTAGG GGCTAGCTACAACGA AACTCTAG | 2123 |
| 1202 | CCUAGCUU A CCAUACUA | 318 | TAGTATGG GGCTAGCTACAACGA AAGCTAGG | 2124 |
| 1207 | CUUACCAU A CUAUAUCU | 319 | AGATATAG GGCTAGCTACAACGA ATGGTAAG | 2125 |
| 1210 | ACCAUACU A UAUCUUUG | 320 | CAAAGATA GGCTAGCTACAACGA AGTATGGT | 2126 |
| 1212 | CAUACUAU A UCUUUGGA | 321 | TCCAAAGA GGCTAGCTACAACGA ATAGTATG | 2127 |
| 1327 | AGAAAAAU A UAUUAUCU | 345 | AGATAATA GGCTAGCTACAACGA ATTTTTCT | 2128 |
| 1329 | AAAAAUAU A UUAUCUCA | 346 | TGAGATAA GGCTAGCTACAACGA ATATTTTT | 2129 |
| 1332 | AAUAUAUU A UCUCAAGU | 348 | ACTTGAGA GGCTAGCTACAACGA AATATATT | 2130 |
| 1341 | UCUCAAGU A UUUUUUAA | 351 | TTAAAAAA GGCTAGCTACAACGA ACTTGAGA | 2131 |
| 1354 | UUAAAAAU A UAUGAAUU | 358 | AATTCATA GGCTAGCTACAACGA ATTTTTAA | 2132 |
| 1356 | AAAAAUAU A UGAAUUCU | 359 | AGAATTCA GGCTAGCTACAACGA ATATTTTT | 2133 |
| 1375 | CUCCAAAU A UUAACUAA | 365 | TTAGTTAA GGCTAGCTACAACGA ATTTGGAG | 2134 |
| 1386 | AACUAAUU A UUAGAUUA | 370 | TAATCTAA GGCTAGCTACAACGA AATTAGTT | 2135 |
| 1394 | AUUAGAUU A UAUUUUGA | 374 | TCAAAATA GGCTAGCTACAACGA AATCTAAT | 2136 |
| 1396 | UAGAUUAU A UUUUGAAA | 375 | TTTCAAAA GGCTAGCTACAACGA ATAATCTA | 2137 |
| 1424 | GCCCAUCU A UUACAUCU | 382 | AGATGTAA GGCTAGCTACAACGA AGATGGGC | 2138 |
| 1427 | CAUCUAUU A CAUCUACA | 384 | TGTAGATG GGCTAGCTACAACGA AATAGATG | 2139 |
| 1433 | UUACAUCU A CAGCUGAC | 386 | GTCAGCTG GGCTAGCTACAACGA AGATGTAA | 2140 |
| 1498 | UCUUAACU A CCUAAUAG | 396 | CTATTAGG GGCTAGCTACAACGA AGTTAAGA | 2141 |
| 1510 | AAUAGCCU A CUAUUGAC | 399 | GTCAATAG GGCTAGCTACAACGA AGGCTATT | 2142 |
| 1513 | AGCCUACU A UUGACCAU | 400 | ATGGTCAA GGCTAGCTACAACGA AGTAGGCT | 2143 |
| 1529 | UAAACCUU A CUGAUAAC | 404 | GTTATCAG GGCTAGCTACAACGA AAGGTTTA | 2144 |
| 1559 | UAACACAU A UUUUGCGU | 410 | ACGCAAAA GGCTAGCTACAACGA ATGTGTTA | 2145 |
| 1571 | UGCGUGUU A UAUGUAUU | 415 | AATACATA GGCTAGCTACAACGA AACACGCA | 2146 |
| 1573 | CGUGUUAU A UGUAUUAU | 416 | ATAATACA GGCTAGCTACAACGA ATAACACG | 2147 |
| 1577 | UUAUAUGU A UUAUACAC | 417 | GTGTATAA GGCTAGCTACAACGA ACATATAA | 2148 |
| 1580 | UAUGUAUU A UACACUAU | 419 | ATAGTGTA GGCTAGCTACAACGA AATACATA | 2149 |
| 1582 | UGUAUUAU A CACUAUAU | 420 | ATATAGTG GGCTAGCTACAACGA ATAATACA | 2150 |
| 1587 | UAUACACU A UAUUCCUA | 421 | TAGGAATA GGCTAGCTACAACGA AGTGTATA | 2151 |
| 1589 | UACACUAU A UUCCUACA | 422 | TGTAGGAA GGCTAGCTACAACGA ATAGTGTA | 2152 |
| 1595 | AUAUUCCU A CAAUAAAG | 425 | CTTTATTG GGCTAGCTACAACGA AGGAATAT | 2153 |
| 1622 | AAAAUGUU A UUUAGAAA | 430 | TTTCTAAA GGCTAGCTACAACGA AACATTTT | 2154 |
| 64 | UCUAUACU G UGAUGAUC | 732 | GATCATCA GGCTAGCTACAACGA AGTATAGA | 2155 |
| 79 | UCACAGCU G CCAAGGCU | 735 | AGCCTTGG GGCTAGCTACAACGA AGCTGTGA | 2156 |
| 121 | AUUUGGCU G CCAGCUUU | 736 | AAAGCTGG GGCTAGCTACAACGA AGCCAAAT | 2157 |
| 168 | GACUUCCU G UCCUGCUG | 738 | CAGOAGGA GGCTAGCTACAACGA AGGAAGTC | 2158 |
| 173 | CCUGUCCU G CUGGUAUC | 739 | GATACCAG GGCTAGCTACAACGA AGGACAGG | 2159 |
| 207 | CCUCACUC G CUCAGCUA | 740 | TAGCTGAG GGCTAGCTACAACGA GAGTGAGG | 2160 |
| 241 | AUUGAAAU G CCUCAACA | 742 | TGTTGAGG GGCTAGCTACAACGA ATTTCAAT | 2161 |
| 288 | CAAUUUCU G UCUCAUCU | 743 | AGATGAGA GGCTAGCTACAACGA AGAAATTG | 2162 |
| 303 | CUUAAUAU G UCUCUUGC | 744 | GCAAGAGA GGCTAGCTACAACGA ATATTAAG | 2163 |
| 310 | UGUCUCUU G CUGAUCUG | 745 | CAGATCAG GGCTAGCTACAACGA AAGAGACA | 2164 |
| 318 | GCUGAUCU G UAUCAUCG | 747 | CGATGATA GGCTAGCTACAACGA AGATCAGC | 2165 |
| 331 | AUCGUGAU G CUUCUCUG | 749 | CAGAGAAG GGCTAGCTACAACGA ATCACGAT | 2166 |
| 347 | GAAGUUCU G CUACAACC | 751 | GGTTGTAG GGCTAGCTACAACGA AGAACTTC | 2167 |
| 365 | CUAGAUCU G CAGCUUGC | 752 | GCAAGCTG GGCTAGCTACAACGA AGATCTAG | 2168 |
| 372 | UGCAGCUU G CCACAUCA | 753 | TGATGTGG GGCTAGCTACAACGA AAGCTGCA | 2169 |
| 392 | UAAAAUCU G UCAUCCCA | 754 | TGGGATGA GGCTAGCTACAACGA AGATTTTA | 2170 |
| 402 | CAUCCCAU G CAGACAGG | 755 | CCTGTCTG GGCTAGCTACAACGA ATGGGATG | 2171 |
| 422 | ACAAUAUU G UAUAACAG | 756 | CTGTTATA GGCTAGCTACAACGA AATATTGT | 2172 |
| 459 | GUUUCUUU G UGAAAAGG | 758 | CCTTTTCA GGCTAGCTACAACGA AAAGAAAC | 2173 |
| 492 | AACUUAUU G UUACCAUA | 760 | TATGGTAA GGCTAGCTACAACGA AATAAGTT | 2174 |
| 502 | UACCAUAU G UAUUCAUC | 761 | GATGAATA GGCTAGCTACAACGA ATATGGTA | 2175 |
| 512 | AUUCAUCU G UUGGAUCU | 762 | AGATCCAA GGCTAGCTACAACGA AGATGAAT | 2176 |
| 522 | UGGAUCUU G UAAACAUG | 763 | CATGTTTA GGCTAGCTACAACGA AAGATCCA | 2177 |
| 570 | AAAUAAGU G UAUAAAAU | 765 | ATTTTATA GGCTAGCTACAACGA ACTTATTT | 2178 |
| 579 | UAUAAAAU G CAACUGUU | 766 | AACAGTTG GGCTAGCTACAACGA ATTTTATA | 2179 |
| 585 | AUGCAACU G UUGAUUUC | 767 | GAAATCAA GGCTAGCTACAACGA AGTTGCAT | 2180 |
| 660 | UUAAAACU G CACUGCCA | 771 | TGGCAGTG GGCTAGCTACAACGA AGTTTTAA | 2181 |
| 665 | ACUGCACU G CCAACAAG | 772 | CTTGTTGG GGCTAGCTACAACGA AGTGCAGT | 2182 |
| 736 | AUUACAAU G UAAAAGCU | 775 | AGCTTTTA GGCTAGCTACAACGA ATTGTAAT | 2183 |
| 807 | AAUGAAGU G UCAUUAUU | 777 | AATAATGA GGCTAGCTACAACGA ACTTCATT | 2184 |
| 844 | UCACAUCU G UUAUCUUA | 779 | TAAGATAA GGCTAGCTACAACGA AGATGTGA | 2185 |
| 869 | AACUAUUU G UAGUAACU | 780 | AGTTACTA GGCTAGCTACAACGA AAATAGTT | 2186 |
| 907 | CAGAAAUU G UAUUUUUU | 781 | AAAAAATA GGCTAGCTACAACGA AATTTCTG | 2187 |
| 920 | UUUUCUAU G CCACAUUA | 782 | TAATGTGG GGCTAGCTACAACGA ATAGAAAA | 2188 |
| 1039 | UAGUACAU G UAGGUAAA | 785 | TTTACCTA GGCTAGCTACAACGA ATGTACTA | 2189 |
| 1058 | AUAAAUCU G UUCUAAGA | 786 | TCTTAGAA GGCTAGCTACAACGA AGATTTAT | 2190 |
| 1102 | GUUAAUAU G UGACAGUG | 789 | CACTGTCA GGCTAGCTACAACGA ATATTAAC | 2191 |
| 1168 | AAGGCACU G UAGUGAAU | 792 | ATTCACTA GGCTAGCTACAACGA AGTGCCTT | 2192 |
| 1253 | AUGAUUUU G CAGGUUGU | 797 | ACAACCTG GGCTAGCTACAACGA AAAATCAT | 2193 |
| 1260 | UGCAGGUU G UCUUCCAU | 798 | ATGGAAGA GGCTAGCTACAACGA AACCTGCA | 2194 |
| 1287 | CAUCCAAU G CAGGCAAG | 799 | CTTGCCTG GGCTAGCTACAACGA ATTGGATG | 2195 |
| 1412 | AUGAACUU G UUGGCCCA | 804 | TGGGCCAA GGCTAGCTACAACGA AAGTTCAT | 2196 |
| 1484 | GUGGGUCC G CAAAAUCU | 808 | AGATTTTG GGCTAGCTACAACGA GGACCCAC | 2197 |
| 1564 | CAUAUUUU G CGUGUUAU | 811 | ATAACACG GGCTAGCTACAACGA AAAATATG | 2198 |
| 1568 | UUUUGCGU G UUAUAUGU | 812 | ACATATAA GGCTAGCTACAACGA ACGCAAAA | 2199 |
| 1575 | UGUUAUAU G UAUUAUAC | 813 | GTATAATA GGCTAGCTACAACGA ATATAACA | 2200 |
| 1619 | GAGAAAAU G UUAUUUAG | 814 | CTAAATAA GGCTAGCTACAACGA ATTTTCTC | 2201 |
| 21 | ACUCCCCA G CUAAACAC | 815 | GTGTTTAG GGCTAGCTACAACGA TGGGGAGT | 2202 |
| 32 | AAACACCC G UAAGACUU | 816 | AAGTCTTA GGCTAGCTACAACGA GGGTGTTT | 2203 |
| 76 | UGAUCACA G CUGCCAAG | 817 | CTTGGCAG GGCTAGCTACAACGA TGTGATCA | 2204 |
| 85 | CUGCCAAG G CUACCUAA | 818 | TTAGGTAG GGCTAGCTACAACGA CTTGGCAG | 2205 |
| 103 | AGAAGACA G UUAUCUCA | 819 | TGAGATAA GGCTAGCTACAACGA TGTCTTCT | 2206 |
| 118 | CAUAUUUG G CUGCCAGC | 820 | GCTGGCAG GGCTAGCTACAACGA CAAATATG | 2207 |
| 125 | GGCUGCCA G CUUUUUAU | 821 | ATAAAAAG GGCTAGCTACAACGA TGGCAGCC | 2208 |
| 177 | UCCUGCUG G UAUCAUGG | 822 | CCATGATA GGCTAGCTACAACGA CAGCAGGA | 2209 |
| 191 | UGGAGAAA G UCCAAUAC | 823 | GTATTGGA GGCTAGCTACAACGA TTTCTCCA | 2210 |
| 212 | CUCGCUCA G CUAUAAGA | 824 | TCTTATAG GGCTAGCTACAACGA TGAGCGAG | 2211 |
| 224 | UAAGAAGA G CCUCAACC | 825 | GGTTGAGG GGCTAGCTACAACGA TCTTCTTA | 2212 |
| 251 | CUCAACAA G CACGUCAA | 826 | TTGACGTG GGCTAGCTACAACGA TTGTTGAG | 2213 |
| 255 | ACAAGCAC G UCAAAAGC | 827 | GCTTTTGA GGCTAGCTACAACGA GTGCTTGT | 2214 |
| 262 | CGUCAAAA G CUACAGAA | 828 | TTCTGTAG GGCTAGCTACAACGA TTTTGACG | 2215 |
| 326 | GUAUCAUC G UGAUGCUU | 829 | AAGCATCA GGCTAGCTACAACGA GATGATAC | 2216 |
| 342 | UCUCUGAA G UUCUGCUA | 830 | TAGCAGAA GGCTAGCTACAACGA TTCAGAGA | 2217 |
| 368 | GAUCUCCA G CUUGCCAC | 831 | GTGGCAAG GGCTAGCTACAACGA TGCAGATC | 2218 |
| 381 | CCACAUCA G CUUAAAAU | 832 | ATTTTAAG GGCTAGCTACAACGA TGATGTGG | 2219 |
| 443 | CUUCCUGA G UAGAAGAG | 833 | CTCTTCTA GGCTAGCTACAACGA TCAGGAAG | 2220 |
| 451 | GUAGAAGA G UUUCUUUG | 834 | CAAAGAAA GGCTAGCTACAACGA TCTTCTAC | 2221 |
| 467 | GUGAAAAG G UCAAGAUU | 835 | AATCTTGA GGCTAGCTACAACGA CTTTTCAC | 2222 |
| 537 | UGAAAAGG G CUUUAUUU | 836 | AAATAAAG GGCTAGCTACAACGA CCTTTTCA | 2223 |
| 568 | CAAAAUAA G UGUAUAAA | 837 | TTTATACA GGCTAGCTACAACGA TTATTTTG | 2224 |
| 603 | UCAACAUG G CUCACAAA | 838 | TTTGTGAG GGCTAGCTACAACGA CATGTTGA | 2225 |
| 644 | GAUGAAGA G UUUAGUUU | 839 | AAACTAAA GGCTAGCTACAACGA TCTTCATC | 2226 |
| 649 | AGAGUUUA G UUUUAAAA | 840 | TTTTAAAA GGCTAGCTACAACGA TAAACTCT | 2227 |
| 673 | GCCAACAA G UUCACUUC | 841 | GAAGTGAA GGCTAGCTACAACGA TTGTTGGC | 2228 |
| 691 | UAUAUAAA G CAUUAUUU | 842 | AAATAATG GGCTAGCTACAACGA TTTATATA | 2229 |
| 713 | CUUUUGAG G UGAAUAUA | 843 | TATATTCA GGCTAGCTACAACGA CTCAAAAG | 2230 |
| 742 | AUGUAAAA G CUUCUUUA | 844 | TAAAGAAG GGCTAGCTACAACGA TTTTACAT | 2231 |
| 758 | AAUACUAA G UAUUUUUC | 845 | GAAAAATA GGCTAGCTACAACGA TTAGTATT | 2232 |
| 769 | UUUUUCAG G UCUUCACC | 846 | GGTGAAGA GGCTAGCTACAACGA CTGAAAAA | 2233 |
| 780 | UUCACCAA G UAUCAAAG | 847 | CTTTGATA GGCTAGCTACAACGA TTGGTGAA | 2234 |
| 788 | GUAUCAAA G UAAUAACA | 848 | TGTTATTA GGCTAGCTACAACGA TTTGATAC | 2235 |
| 805 | CAAAUGAA G UGUCAUUA | 849 | TAATGACA GGCTAGCTACAACGA TTCATTTG | 2236 |
| 823 | UCAAAAUA G UCCACUGA | 850 | TCAGTGGA GGCTAGCTACAACGA TATTTTGA | 2237 |
| 872 | UAUUUGUA G UAACUAUC | 851 | GATAGTTA GGCTAGCTACAACGA TACAAATA | 2238 |
| 941 | CUUUUAAA G UUGAUGAG | 852 | CTCATCAA GGCTAGCTACAACGA TTTAAAAG | 2239 |
| 956 | AGAAUCAA G UAUGGAAA | 853 | TTTCCATA GGCTAGCTACAACGA TTGATTCT | 2240 |
| 966 | AUGGAAAA G UAAGGCCA | 854 | TGGCCTTA GGCTAGCTACAACGA TTTTCCAT | 2241 |
| 971 | AAAGUAAG G CCAUACUC | 855 | GAGTATGG GGCTAGCTACAACGA CTTACTTT | 2242 |
| 1003 | CCUUUUAA G UAAUUUUU | 856 | AAAAATTA GGCTAGCTACAACGA TTAAAAGG | 2243 |
| 1033 | GAAUUCUA G UACAUGUA | 857 | TACATGTA GGCTAGCTACAACGA TAGAATTC | 2244 |
| 1043 | ACAUGUAG G UAAAUCAU | 858 | ATGATTTA GGCTAGCTACAACGA CTACATGT | 2245 |
| 1091 | GAGAACUG G UGGUUAAU | 859 | ATTAACCA GGCTAGCTACAACGA CAGTTCTC | 2246 |
| 1094 | AACUGGUG G UUAAUAUG | 860 | CATATTAA GGCTAGCTACAACGA CACCAGTT | 2247 |
| 1108 | AUGUGACA G UGAGAUUA | 861 | TAATCTCA GGCTAGCTACAACGA TGTCACAT | 2248 |
| 1117 | UGAGAUUA G UCAUAUCA | 862 | TGATATGA GGCTAGCTACAACGA TAATCTCA | 2249 |
| 1163 | CAUUUAAG G CACUGUAG | 863 | CTACAGTG GGCTAGCTACAACGA CTTAAATG | 2250 |
| 1171 | GCACUGUA G UGAAUUAU | 864 | ATAATTCA GGCTAGCTACAACGA TACAGTGC | 2251 |
| 1184 | UUAUCUGA G CUAGAGUU | 865 | AACTCTAG GGCTAGCTACAACGA TCAGATAA | 2252 |
| 1190 | GAGCUAGA G UUACCUAG | 866 | CTAGGTAA GGCTAGCTACAACGA TCTAGCTC | 2253 |
| 1198 | GUUACCUA G CUUACCAU | 867 | ATGGTAAG GGCTAGCTACAACGA TAGGTAAC | 2254 |
| 1257 | UUUUGCAG G UUGUCUUC | 868 | GAAGACAA GGCTAGCTACAACGA CTGCAAAA | 2255 |
| 1273 | CCAUUCCA G CCUAACAU | 869 | ATGTTAGG GGCTAGCTACAACGA TGGAATGG | 2256 |
| 1291 | CAAUGCAG G CAAGGAAA | 870 | TTTCCTTG GGCTAGCTACAACGA CTGCATTG | 2257 |
| 1314 | GAUUUCCA G UGACAGAA | 871 | TTCTGTCA GGCTAGCTACAACGA TGGAAATC | 2258 |
| 1339 | UAUCUCAA G UAUUUUUU | 872 | AAAAAATA GGCTAGCTACAACGA TTGAGATA | 2259 |
| 1416 | ACUUGUUG G CCCAUCUA | 873 | TAGATGGG GGCTAGCTACAACGA CAACAAGT | 2260 |
| 1436 | CAUCUACA G CUGACCCU | 874 | AGGGTCAG GGCTAGCTACAACGA TGTAGATG | 2261 |
| 1456 | ACAUGGOG G UUAGGGGA | 875 | TCCCCTAA GGCTAGCTACAACGA CCCCATGT | 2262 |
| 1465 | UUAGGGGA G CUGACAAU | 876 | ATTGTCAG GGCTAGCTACAACGA TCCCCTAA | 2263 |
| 1476 | GACAAUUC G UGGGUCCG | 877 | CGGACCCA GGCTAGCTACAACGA GAATTGTC | 2264 |
| 1480 | AUUCGUGG G UCCGCAAA | 878 | TTTGCGGA GGCTAGCTACAACGA CCACGAAT | 2265 |
| 1506 | ACCUAAUA G CCUACUAU | 879 | ATAGTAGG GGCTAGCTACAACGA TATTAGGT | 2266 |
| 1545 | CAUAAACA G UAAAUUAA | 880 | TTAATTTA GGCTAGCTACAACGA TGTTTATG | 2267 |
| 1566 | UAUUUUGC G UGUUAUAU | 881 | ATATAACA GGCTAGCTACAACGA GCAAAATA | 2268 |
| 1603 | ACAAUAAA G UAAGCUAG | 882 | CTAGCTTA GGCTAGCTACAACGA TTTATTGT | 2269 |
| 1607 | UAAAGUAA G CUAGAGAA | 883 | TTCTCTAG GGCTAGCTACAACGA TTACTTTA | 2270 |
| 13 | GUCAGAAA A CUCCCCAG | 884 | CTGGGGAG GGCTAGCTACAACGA TTTCTGAC | 2271 |
| 26 | CCAGCUAA A CACCCGUA | 885 | TACGGGTG GGCTAGCTACAACGA TTAGCTGG | 2272 |
| 28 | AGCUAAAC A CCCGUAAG | 886 | CTTACGGG GGCTAGCTACAACGA GTTTAGCT | 2273 |
| 37 | CCCGUAAG A CUUCAUAC | 887 | GTATGAAG GGCTAGCTACAACGA CTTACGGG | 2274 |
| 42 | AAGACUUC A UACAACAC | 888 | GTGTTGTA GGCTAGCTACAACGA GAAGTCTT | 2275 |
| 47 | UUCAUACA A CACAAUAC | 889 | GTATTGTG GGCTAGCTACAACGA TGTATGAA | 2276 |
| 49 | CAUACAAC A CAAUACUC | 890 | GAGTATTG GGCTAGCTACAACGA GTTGTATG | 2277 |
| 52 | ACAACACA A UACUCUAU | 891 | ATAGAGTA GGCTAGCTACAACGA TGTGTTGT | 2278 |
| 67 | AUACUGUG A UGAUCACA | 892 | TGTGATCA GGCTAGCTACAACGA CACAGTAT | 2279 |
| 70 | CUGUGAUG A UCACAGCU | 893 | AGCTGTGA GGCTAGCTACAACGA CATCACAG | 2280 |
| 73 | UGAUGAUC A CAGCUGCC | 894 | GGCAGCTG GGCTAGCTACAACGA GATCATCA | 2281 |
| 100 | AAAAGAAG A CAGUUAUC | 895 | GATAACTG GGCTAGCTACAACGA CTTCTTTT | 2282 |
| 111 | GUUAUCUC A UAUUUGGC | 896 | GCCAAATA GGCTAGCTACAACGA GAGATAAC | 2283 |
| 144 | UUCUCUCG A CCACUUAA | 897 | TTAAGTGG GGCTAGCTACAACGA CGAGAGAA | 2284 |
| 147 | UCUCGACC A CUUAAAAC | 898 | GTTTTAAG GGCTAGCTACAACGA GGTCGAGA | 2285 |
| 154 | CACUUAAA A CUUCAGAC | 899 | GTCTGAAG GGCTAGCTACAACGA TTTAAGTG | 2286 |
| 161 | AACUUCAG A CUUCCUGU | 900 | ACAGGAAG GGCTAGCTACAACGA CTGAAGTT | 2287 |
| 182 | CUGGUAUC A UGGAGAAA | 901 | TTTCTCCA GGCTAGCTACAACGA GATACCAG | 2288 |
| 196 | AAAGUCCA A UACCUCAC | 902 | GTGAGGTA GGCTAGCTACAACGA TGGACTTT | 2289 |
| 203 | AAUACCUC A CUCGCUCA | 903 | TGAGCGAG GGCTAGCTACAACGA GAGGTATT | 2290 |
| 230 | GAGCCUCA A CCAUUGAA | 904 | TTCAATGG GGCTAGCTACAACGA TGAGGCTC | 2291 |
| 233 | CCUCAACC A UUGAAAUG | 905 | CATTTCAA GGCTAGCTACAACGA GGTTGAGG | 2292 |
| 239 | CCAUUGAA A UGCCUCAA | 906 | TTGAGGCA GGCTAGCTACAACGA TTCAATGG | 2293 |
| 247 | AUGCCUCA A CAAGCACG | 907 | CGTGCTTG GGCTAGCTACAACGA TGAGGCAT | 2294 |
| 253 | CAACAAGC A CGUCAAAA | 908 | TTTTGACG GGCTAGCTACAACGA GCTTGTTG | 2295 |
| 270 | GCUACAGA A UCUAUUUA | 909 | TAAATAGA GGCTAGCTACAACGA TCTGTAGC | 2296 |
| 282 | AUUUAUCA A UUUCUGUC | 910 | GACAGAAA GGCTAGCTACAACGA TGATAAAT | 2297 |
| 293 | UCUGUCUC A UCUUAAUA | 911 | TATTAAGA GGCTAGCTACAACGA GAGACAGA | 2298 |
| 299 | UCAUCUUA A UAUGUCUC | 912 | GAGACATA GGCTAGCTACAACGA TAAGATGA | 2299 |
| 314 | UCUUGCUG A UCUGUAUC | 913 | GATACAGA GGCTAGCTACAACGA CAGCAAGA | 2300 |
| 323 | UCUGUAUC A UCGUGAUG | 914 | CATCACGA GGCTAGCTACAACGA GATACAGA | 2301 |
| 329 | UCAUCGUG A UGCUUCUC | 915 | GAGAAGCA GGCTAGCTACAACGA CACGATGA | 2302 |
| 353 | CUGCUACA A CCUCUAGA | 916 | TCTAGAGG GGCTAGCTACAACGA TGTAGCAG | 2303 |
| 361 | ACCUCUAG A UCUGCAGC | 917 | GCTGCAGA GGCTAGCTACAACGA CTAGAGGT | 2304 |
| 375 | AGCUUGCC A CAUCAGCU | 918 | AGCTGATG GGCTAGCTACAACGA GGCAAGCT | 2305 |
| 377 | CUUGCCAC A UCAGCUUA | 919 | TAAGCTGA GGCTAGCTACAACGA GTGGCAAG | 2306 |
| 388 | AGCUUAAA A UCUGUCAU | 920 | ATGACAGA GGCTAGCTACAACGA TTTAAGCT | 2307 |
| 395 | AAUCUGUC A UCCCAUGC | 921 | GCATGGGA GGCTAGCTACAACGA GACAGATT | 2308 |
| 400 | GUCAUCCC A UGGAGACA | 922 | TGTCTGCA GGCTAGCTACAACGA GGGATGAC | 2309 |
| 406 | CCAUGCAG A CAGGAAAA | 923 | TTTTCCTG GGCTAGCTACAACGA CTGCATGG | 2310 |
| 414 | ACAGGAAA A CAAUAUUG | 924 | CAATATTG GGCTAGCTACAACGA TTTCCTGT | 2311 |
| 417 | GGAAAACA A UAUUGUAU | 925 | ATACAATA GGCTAGCTACAACGA TGTTTTCC | 2312 |
| 427 | AUUGUAUA A CAGACCAC | 926 | GTGGTCTG GGCTAGCTACAACGA TATACAAT | 2313 |
| 431 | UAUAACAG A CCACUUCC | 927 | GGAAGTGG GGCTAGCTACAACGA CTGTTATA | 2314 |
| 434 | AACAGACC A CUUCCUGA | 928 | TCAGGAAG GGCTAGCTACAACGA GGTCTGTT | 2315 |
| 473 | AGGUCAAG A UUAAGACU | 929 | AGTCTTAA GGCTAGCTACAACGA CTTGACCT | 2316 |
| 479 | AGAUUAAG A CUAAAACU | 930 | AGTTTTAG GGCTAGCTACAACGA CTTAATCT | 2317 |
| 485 | AGACUAAA A CUUAUUGU | 931 | ACAATAAG GGCTAGCTACAACGA TTTAGTCT | 2318 |
| 498 | UUGUUACC A UAUGUAUU | 932 | AATACATA GGCTAGCTACAACGA GGTAACAA | 2319 |
| 508 | AUGUAUUC A UCUGUUGG | 933 | CCAACAGA GGCTAGCTACAACGA GAATACAT | 2320 |
| 517 | UCUGUUGG A UCUUGUAA | 934 | TTACAAGA GGCTAGCTACAACGA CCAACAGA | 2321 |
| 526 | UCUUGUAA A CAUGAAAA | 935 | TTTTCATG GGCTAGCTACAACGA TTACAAGA | 2322 |
| 528 | UUGUAAAC A UGAAAAGG | 936 | CCTTTTCA GGCTAGCTACAACGA GTTTACAA | 2323 |
| 552 | UUUCAAAA A UUAACUUC | 937 | GAAGTTAA GGCTAGCTACAACGA TTTTGAAA | 2324 |
| 556 | AAAAAUUA A CUUCAAAA | 938 | TTTTGAAG GGCTAGCTACAACGA TAATTTTT | 2325 |
| 564 | ACUUCAAA A UAAGUGUA | 939 | TACACTTA GGCTAGCTACAACGA TTTGAAGT | 2326 |
| 577 | UGUAUAAA A UGCAACUG | 940 | CAGTTGCA GGCTAGCTACAACGA TTTATACA | 2327 |
| 582 | AAAAUGCA A CUGUUGAU | 941 | ATCAACAG GGCTAGCTACAACGA TGCATTTT | 2328 |
| 589 | AACUGUUG A UUUCCUCA | 942 | TGAGGAAA GGCTAGCTACAACGA CAACAGTT | 2329 |
| 598 | UUUCCUCA A CAUGGCUC | 943 | GAGCCATG GGCTAGCTACAACGA TGAGGAAA | 2330 |
| 600 | UCCUCAAC A UGGCUCAC | 944 | GTGAGCCA GGCTAGCTACAACGA GTTGAGGA | 2331 |
| 607 | CAUGGCUC A CAAAUUUC | 945 | GAAATTTG GGCTAGCTACAACGA GAGCCATG | 2332 |
| 611 | GCUCACAA A UUUCUAUC | 946 | GATAGAAA GGCTAGCTACAACGA TTGTGAGC | 2333 |
| 624 | UAUCCCAA A UCUUUUCU | 947 | AGAAAAGA GGCTAGCTACAACGA TTGGGATA | 2334 |
| 637 | UUCUGAAG A UGAAGAGU | 948 | ACTCTTCA GGCTAGCTACAACGA CTTCAGAA | 2335 |
| 657 | GUUUUAAA A CUGCACUG | 949 | CAGTGCAG GGCTAGCTACAACGA TTTAAAAC | 2336 |
| 662 | AAAACUGC A CUGCCAAC | 950 | GTTGGCAG GGCTAGCTACAACGA GCAGTTTT | 2337 |
| 669 | CACUGCCA A CAAGUUCA | 951 | TGAACTTG GGCTAGCTACAACGA TGGCAGTG | 2338 |
| 677 | ACAAGUUC A CUUCAUAU | 952 | ATATGAAG GGCTAGCTACAACGA GAACTTGT | 2339 |
| 682 | UUCACUUC A UAUAUAAA | 953 | TTTATATA GGCTAGCTACAACGA GAAGTGAA | 2340 |
| 693 | UAUAAAGC A UUAUUUUU | 954 | AAAAATAA GGCTAGCTACAACGA GCTTTATA | 2341 |
| 717 | UGAGGUGA A UAUAAUUU | 955 | AAATTATA GGCTAGCTACAACGA TCACCTCA | 2342 |
| 722 | UGAAUAUA A UUUAUAUU | 956 | AATATAAA GGCTAGCTACAACGA TATATTCA | 2343 |
| 734 | AUAUUACA A UGUAAAAG | 957 | CTTTTACA GGCTAGCTACAACGA TGTAATAT | 2344 |
| 751 | CUUCUUUA A UACUAAGU | 958 | ACTTAGTA GGCTAGCTACAACGA TAAAGAAG | 2345 |
| 775 | AGGUCUUC A CCAAGUAU | 959 | ATACTTGG GGCTAGCTACAACGA GAAGACCT | 2346 |
| 791 | UCAAAGUA A UAACACAA | 960 | TTGTGTTA GGCTACCTACAACGA TACTTTGA | 2347 |
| 794 | AAGUAAUA A CACAAAUG | 961 | CATTTGTG GGCTAGCTACAACGA TATTACTT | 2348 |
| 796 | GUAAUAAC A CAAAUCAA | 962 | TTCATTTG GGCTAGCTACAACGA GTTATTAC | 2349 |
| 800 | UAACACAA A UGAAGUGU | 963 | ACACTTCA GGCTAGCTACAACGA TTGTGTTA | 2350 |
| 810 | GAAGUGUC A UUAUUCAA | 964 | TTGAATAA GGCTAGCTACAACGA GACACTTC | 2351 |
| 820 | UAUUCAAA A UAGUCCAC | 965 | GTGGACTA GGCTAGCTACAACGA TTTGAATA | 2352 |
| 827 | AAUAGUCC A CUGACUCC | 966 | GGAGTCAG GGCTAGCTACAACGA GGACTATT | 2353 |
| 831 | GUCCACUG A CUCCUCAC | 967 | GTGAGGAG GGCTAGCTACAACGA CAGTGGAC | 2354 |
| 838 | GACUCCUC A CAUCUGUU | 968 | AACAGATG GGCTAGCTACAACGA GAGGAGTC | 2355 |
| 840 | CUCCUCAC A UCUGUUAU | 969 | ATAACAGA GGCTAGCTACAACGA GTGAGGAG | 2356 |
| 862 | UAUAAAGA A CUAUUUGU | 970 | ACAAATAG GGCTAGCTACAACGA TCTTTATA | 2357 |
| 875 | UUGUAGUA A CUAUCAGA | 971 | TCTGATAG GGCTAGCTACAACGA TACTACAA | 2358 |
| 884 | CUAUCAGA A UCUACAUU | 972 | AATGTAGA GGCTAGCTACAACGA TCTGATAG | 2359 |
| 890 | GAAUCUAC A UUCUAAAA | 973 | TTTTAGAA GGCTAGCTACAACGA GTAGATTC | 2360 |
| 898 | AUUCUAAA A CAGAAAUU | 974 | AATTTCTG GGCTAGCTACAACGA TTTAGAAT | 2361 |
| 904 | AAACAGAA A UUGUAUUU | 975 | AAATACAA GGCTAGCTACAACGA TTCTGTTT | 2362 |
| 923 | UCUAUGCC A CAUUAACA | 976 | TGTTAATG GGCTAGCTACAACGA GGCATAGA | 2363 |
| 925 | UAUGCCAC A UUAACAUC | 977 | GATGTTAA GGCTAGCTACAACGA GTGGCATA | 2364 |
| 929 | CCACAUUA A CAUCUUUU | 978 | AAAAGATG GGCTAGCTACAAZGA TAATGTGG | 2365 |
| 931 | ACAUUAAC A UCUUUUAA | 979 | TTAAAAGA GGCTAGCTACAACGA GTTAATGT | 2366 |
| 945 | UAAAGUUG A UGAGAAUC | 980 | GATTCTCA GGCTAGCTACAACGA CAACTTTA | 2367 |
| 951 | UGAUGAGA A UCAAGUAU | 981 | ATACTTGA GGCTAGCTACAACGA TCTCATCA | 2368 |
| 974 | GUAAGGCC A UACUCUUA | 982 | TAAGAGTA GGCTAGCTACAACGA GGCCTTAC | 2369 |
| 984 | ACUCUUAC A UAAUAAAA | 983 | TTTTATTA GGCTAGCTACAACGA GTAAGAGT | 2370 |
| 987 | CUUACAUA A UAAAAUUC | 984 | GAATTTTA GGCTAGCTACAACGA TATGTAAG | 2371 |
| 992 | AUAAUAAA A UUCCUUUU | 985 | AAAAGGAA GGCTAGCTACAACGA TTTATTAT | 2372 |
| 1006 | UUUAAGUA A UUUUUUCA | 986 | TGAAAAAA GGCTAGCTACAACGA TACTTAAA | 2373 |
| 1019 | UUCAAAGA A UCACAGAA | 987 | TTCTGTGA GGCTAGCTACAACGA TCTTTGAA | 2374 |
| 1022 | AAAGAAUC A CAGAAUUC | 988 | GAATTCTG GGCTAGCTACAACGA GATTCTTT | 2375 |
| 1027 | AUCACAGA A UUCUAGUA | 989 | TACTAGAA GGCTAGCTACAACGA TCTGTGAT | 2376 |
| 1037 | UCUAGUAC A UGUAGGUA | 990 | TACCTACA GGCTAGCTACAACGA GTACTAGA | 2377 |
| 1047 | GUAGGUAA A UCAUAAAU | 991 | ATTTATGA GGCTAGCTACAACGA TTACCTAC | 2378 |
| 1050 | GGUAAAUC A UAAAUCUG | 992 | CAGATTTA GGCTAGCTACAACGA GATTTACC | 2379 |
| 1054 | AAUCAUAA A UCUGUUCU | 993 | AGAACAGA GGCTAGCTACAACGA TTATGATT | 2380 |
| 1066 | GUUCUAAG A CAUAUGAU | 994 | ATCATATG GGCTAGCTACAACGA CTTAGAAC | 2381 |
| 1068 | UCUAAGAC A UAUGAUCA | 995 | TGATCATA GGCTAGCTACAACGA GTCTTAGA | 2382 |
| 1073 | GACAUAUG A UCAACAGA | 998 | TCTGTTGA GGCTAGCTACAACGA CATATGTC | 2383 |
| 1077 | UAUGAUCA A CAGAUGAG | 997 | CTCATCTG GGCTAGCTACAACGA TGATCATA | 2384 |
| 1081 | AUCAACAG A UGAGAACU | 998 | AGTTCTCA GGCTAGCTACAACGA CTGTTGAT | 2385 |
| 1087 | AGAUGAGA A CUGGUGGU | 999 | ACCACCAG GGCTAGCTACAACGA TCTCATCT | 2386 |
| 1098 | GGUGGUUA A UAUGUGAC | 1000 | GTCACATA GGCTAGCTACAACGA TAACCACC | 2387 |
| 1105 | AAUAUGUG A CAGUGAGA | 1001 | TCTCACTG GGCTAGCTACAACGA CACATATT | 2388 |
| 1113 | ACAGUGAG A UUAGUCAU | 1002 | ATGACTAA GGCTAGCTACAACGA CTCACTGT | 2389 |
| 1120 | GAUUAGUC A UAUCACUA | 1003 | TAGTGATA GGCTAGCTACAACGA GACTAATC | 2390 |
| 1125 | GUCAUAUC A CUAAUAUA | 1004 | TATATTAG GGCTAGCTACAACGA GATATGAC | 2391 |
| 1129 | UAUCACUA A UAUACUAA | 1005 | TTAGTATA GGCTAGCTACAACGA TAGTGATA | 2392 |
| 1137 | AUAUACUA A CAACAGAA | 1006 | TTCTGTTG GGCTAGCTACAACGA TAGTATAT | 2393 |
| 1140 | UACUAACA A CAGAAUCU | 1007 | AGATTCTG GGCTAGCTACAACGA TGTTAGTA | 2394 |
| 1145 | ACAACAGA A UCUAAUCU | 1008 | AGATTAGA GGCTAGCTACAACGA TCTGTTGT | 2395 |
| 1150 | AGAAUCUA A UCUUCAUU | 1009 | AATGAAGA GGCTAGCTACAACGA TAGATTCT | 2396 |
| 1156 | UAAUCUUC A UUUAAGGC | 1010 | GCCTTAAA GGCTAGCTACAACGA GAAGATTA | 2397 |
| 1165 | UUUAAGGC A CUGUAGUG | 1011 | CACTACAG GGCTAGCTACAACGA GCCTTAAA | 2398 |
| 1175 | UGUAGUGA A UUAUCUGA | 1012 | TCAGATAA GGCTAGCTACAACGA TCACTACA | 2399 |
| 1205 | AGCUUACC A UACUAUAU | 1013 | ATATAGTA GGCTAGCTACAACGA GGTAAGCT | 2400 |
| 1221 | UCUUUGGA A UCAUGAAA | 1014 | TTTCATGA GGCTAGCTACAACGA TCCAAAGA | 2401 |
| 1224 | UUGGAAUC A UGAAACCU | 1015 | AGGTTTCA GGCTAGCTACAACGA GATTCCAA | 2402 |
| 1229 | AUCAUGAA A CCUUAAGA | 1016 | TCTTAAGG GGCTAGCTACAACGA TTCATGAT | 2403 |
| 1237 | ACCUUAAG A CUUCAGAA | 1017 | TTCTGAAG GGCTAGCTACAACGA CTTAAGGT | 2404 |
| 1245 | ACUUCAGA A UGAUUUUG | 1018 | CAAAATCA GGCTAGCTACAACGA TCTGAAGT | 2405 |
| 1248 | UCAGAAUG A UUUUGCAG | 1019 | CTGCAAAA GGCTAGCTACAACGA CATTCTGA | 2406 |
| 1267 | UGUCUUCC A UUCCAGCC | 1020 | GGCTGGAA GGCTAGCTACAACGA GGAAGACA | 2407 |
| 1278 | CCAGCCUA A CAUCCAAU | 1021 | ATTGGATG GGCTAGCTACAACGA TAGGCTGG | 2408 |
| 1280 | AGCCUAAC A UCCAAUGC | 1022 | GCATTGGA GGCTAGCTACAACGA GTTAGGCT | 2409 |
| 1285 | AACAUCCA A UGCAGGCA | 1023 | TGCCTGCA GGCTAGCTACAACGA TGGATGTT | 2410 |
| 1300 | CAAGGAAA A UAAAAGAU | 1024 | ATCTTTTA GGCTAGCTACAACGA TTTCCTTG | 2411 |
| 1307 | AAUAAAAG A UUUCCAGU | 1025 | ACTGGAAA GGCTAGCTACAACGA CTTTTATT | 2412 |
| 1317 | UUCCAGUG A CAGAAAAA | 1026 | TTTTTCTG GGCTAGCTACAACGA CACTGGAA | 2413 |
| 1325 | ACAGAAAA A UAUAUUAU | 1027 | ATAATATA GGCTAGCTACAACGA TTTTCTGT | 2414 |
| 1352 | UUUUAAAA A UAUAUGAA | 1028 | TTCATATA GGCTAGCTACAACGA TTTTAAAA | 2415 |
| 1360 | AUAUAUGA A UUCUCUCU | 1029 | AGAGAGAA GGCTAGCTACAACGA TCATATAT | 2416 |
| 1373 | CUCUCCAA A UAUUAACU | 1030 | AGTTAATA GGCTAGCTACAACGA TTGGAGAG | 2417 |
| 1379 | AAAUAUUA A CUAAUUAU | 1031 | ATAATTAG GGCTAGCTACAACGA TAATATTT | 2418 |
| 1383 | AUUAACUA A UUAUUAGA | 1032 | TCTAATAA GGCTAGCTACAACGA TAGTTAAT | 2419 |
| 1391 | AUUAUUAG A UUAUAUUU | 1033 | AAATATAA GGCTAGCTACAACGA CTAATAAT | 2420 |
| 1404 | AUUUUGAA A UGAACUUG | 1034 | CAAGTTCA GGCTAGCTACAACGA TTCAAAAT | 2421 |
| 1408 | UGAAAUGA A CUUGUUGG | 1035 | CCAACAAG GGCTAGCTACAACGA TCATTTCA | 2422 |
| 1420 | GUUGGCCC A UCUAUUAC | 1036 | GTAATAGA GGCTAGCTACAACGA GGGCCAAC | 2423 |
| 1429 | UCUAUUAC A UCUACAGC | 1037 | GCTGTAGA GGCTAGCTACAACGA GTAATAGA | 2424 |
| 1440 | UACAGCUG A CCCUUGAA | 1038 | TTCAAGGG GGCTAGCTACAACGA CAGCTGTA | 2425 |
| 1448 | ACCCUUGA A CAUGGGGG | 1039 | CCCCCATG GGCTAGCTACAACGA TCAAGGGT | 2426 |
| 1450 | CCUUGAAC A UGGGGGUU | 1040 | AACCCCCA GGCTAGCTACAACGA GTTCAAGG | 2427 |
| 1469 | GGGAGCUG A CAAUUCGU | 1041 | ACGAATTG GGCTAGCTACAACGA CAGCTCCC | 2428 |
| 1472 | AGCUGACA A UUCGUGGG | 1042 | CCCACGAA GGCTAGCTACAACGA TGTCAGCT | 2429 |
| 1489 | UCCGCAAA A UCUUAACU | 1043 | AGTTAAGA GGCTAGCTACAACGA TTTGCGGA | 2430 |
| 1495 | AAAUCUUA A CUACCUAA | 1044 | TTAGGTAG GGCTAGCTACAACGA TAAGATTT | 2431 |
| 1503 | ACUACCUA A UAGCCUAC | 1045 | GTAGGCTA GGCTAGCTACAACGA TAGGTAGT | 2432 |
| 1517 | UACUAUUG A CCAUAAAC | 1046 | GTTTATGG GGCTAGCTACAACGA CAATAGTA | 2433 |
| 1520 | UAUUGACC A UAAACCUU | 1047 | AAGGTTTA GGCTAGCTACAACGA GGTCAATA | 2434 |
| 1524 | GACCAUAA A CCUUACUG | 1048 | CAGTAAGG GGCTAGCTACAACGA TTATGGTC | 2435 |
| 1533 | CCUUACUG A UAACAUAA | 1049 | TTATGTTA GGCTAGCTACAACGA CAGTAAGG | 2436 |
| 1536 | UACUGAUA A CAUAAACA | 1050 | TGTTTATG GGCTAGCTACAACGA TATCAGTA | 2437 |
| 1538 | CUGAUAAC A UAAACAGU | 1051 | ACTGTTTA GGCTAGCTACAACGA GTTATCAG | 2438 |
| 1542 | UAACAUAA A CAGUAAAU | 1052 | ATTTACTG GGCTAGCTACAACGA TTATGTTA | 2439 |
| 1549 | AACAGUAA A UUAACACA | 1053 | TGTGTTAA GGCTAGCTACAACGA TTACTGTT | 2440 |
| 1553 | GUAAAUUA A CACAUAUU | 1054 | AATATGTG GGCTAGCTACAACGA TAATTTAC | 2441 |
| 1555 | AAAUUAAC A CAUAUUUU | 1055 | AAAATATG GGCTAGCTACAACGA GTTAATTT | 2442 |
| 1557 | AUUAACAC A UAUUUUGC | 1056 | GCAAAATA GGCTAGCTACAACGA GTGTTAAT | 2443 |
| 1584 | UAUUAUAC A CUAUAUUU | 1057 | GAATATAG GGCTAGCTACAACGA GTATAATA | 2444 |
| 1598 | UUCCUACA A UAAAGUAA | 1058 | TTACTTTA GGCTAGCTACAACGA TGTAGGAA | 2445 |
| 1617 | UAGAGAAA A UGUUAUUU | 1059 | AAATAACA GGCTAGCTACAACGA TTTCTCTA | 2446 |

Input Sequence = PLN. Cut Site = RN
Stem Length = 8. Core Sequence = GGCTAGCTACAACGA
PLN (Homo sapiens phospholamban (PLN) mRNA.; 1635 bp)

TABLE VIII
Human Phospholamban (PLN) amberzyme Ribozme and Target Sequence

| Pos | Substrate | Seq ID | Ribozyme | Rz Seq ID |
|---|---|---|---|---|
| 64 | UCUAUACU G UGAUGAUC | 732 | GAUCAUCA GGAGGAAACUCC CU UCAAGGACAUCGUCCGGG AGUAUAGA | 2447 |
| 66 | UAVACUGU G AUGAUCAC | 733 | GUGAUCAU GGAGGAAACUCC CU UCAAGGACAUCGUCCGGG ACAGUAUA | 2448 |
| 69 | ACUGUGAU G AUCACAGC | 734 | GCUGUGAU GGAGGAAACUCC CU UCAAGGACAUCGUCCGGG AUCACAGU | 2449 |
| 79 | UCACAGCU G CCAAGGCU | 735 | AGCCUUGG GGAGGAAACUCC CU UCAAGGACAUCGUCCGGG AGCUGUGA | 2450 |
| 121 | AUUUGGCU G CCAGCUUU | 736 | AAAGCUGG GGAGGAAACUCC CU UCAAGGACAUCGUCCGGG AGCCAAAU | 2451 |
| 143 | UUUCUCUC G ACCACUUA | 737 | UAAGUGGU GGAGGAAACUCC CU UCAAGGACAUCGUCCGGG GAGAGAAA | 2452 |
| 168 | GACUUCCU G UCCUCCUC | 738 | CACCAGGA GGAGGAAACUCC CU UCAAGGACAUCGUCCGGG AGGAAGUC | 2453 |
| 173 | CCUGUCCU G CUGGUAUC | 739 | GAUACCAG GGAGGAAACUCC CU UCAAGGACAUCGUCCGGG AGGACAGG | 2454 |
| 207 | CCUCACUC G CUCAGCUA | 740 | UAGCUGAG GGAGGAAACUCC CU UCAAGGACAUCGUCCGGG GAGUGAGG | 2455 |
| 236 | CAACCAUU G AAAUGCCU | 741 | AGGCAUUU GGAGGAAACUCC CU UCAAGGACAUCGUCCGGG AAUGGUUG | 2456 |
| 241 | AUUGAAAU G CCUCAACA | 742 | UGUUGAGG GGAGGAAACUCC CU UCAAGGACAUCGUCCGGG AUUUCAAU | 2457 |
| 288 | CAAUUUCU G UCUCAUCU | 743 | AGAUGAGA GCAGGAAACUCC CU UCAAGGACAUCGUCCGGG AGAAAUUG | 2458 |
| 303 | CUUAAUAU G UCUCUUGC | 744 | GCAAGAGA CGACGAAACUCC CU UCAAGCACAUCCUCCGGG AUAUUAAC | 2459 |
| 310 | UGUCUCUU G CUGAUCUC | 745 | CACAUCAC GGACGAAACUCC CU UCAAGGACAUCGUCCCGG AAGAGACA | 2460 |
| 313 | CUCUUCCU G AUCUGUAU | 746 | AUACAGAU CGAGGAAACUCC CU UCAAGGACAUCGUCCGGC AGCAAGAC | 2461 |
| 318 | GCUGAUCU G UAUCAUCC | 747 | CGAUCAUA CCAGCAAACUCC CU UCAAGGACAUCGUCCGGG AGAUCAGC | 2462 |
| 328 | AUCAUCCU G AUGCUUCU | 748 | AGAAGCAU GGAGCAAACUCC CU UCAAGGACAUCGUCCGGC ACGAUGAU | 2463 |
| 331 | AUCCUCAU G CUUCUCUG | 749 | CAGACAAC GGAGCAAACUCC CU UCAAGCACAUCCUCCGGG AUCACGAU | 2464 |
| 339 | GCUUCUCU G AAGUUCUG | 750 | CAGAACUU GGAGGAAACUCC CU UCAAGGACAUCGUCCGGG ACACAAGC | 2465 |
| 347 | CAACUUCU G CUACAACC | 751 | GGUUCUAG GGAGGAAACUCC CU UCAAGGACAUCCUCCGGG AGAACUUC | 2466 |
| 365 | CUAGAUCU G CACCUUCC | 752 | GCAAGCUC CGACGAAACUCC CU UCAAGGACAUCGUCCGGG AGAUCUAG | 2467 |
| 372 | UGCAGCUU G CCACAUCA | 753 | UGAUCUGG CGAGGAAACUCC CU UCAAGGACAUCGUCCGGC AAGCUCCA | 2468 |
| 392 | UAAAAUCU G UCAUCCCA | 754 | UCGGAUGA GGACGAAACUCC CU UCAAGCACAUCCUCCCCG AGAUUUUA | 2469 |
| 402 | CAUCCCAU G CAGACAGG | 755 | CCUGUCUG CGAGCAAACUCC CU UCAACGACAUCCUCCCCG AUGGGAUG | 2470 |
| 422 | ACAAUAUU G UAUAACAG | 756 | CUGUUAUA CCACGAAACUCC CU UCAAGGACAUCCUCCGCC AAUAUUGU | 2471 |
| 441 | CACUUCCU G ACUAGAAG | 757 | CUUCUACU CCACCAAACUCC CU UCAAGGACAUCGUCCGGG ACGAAGUC | 2472 |
| 459 | GUUUCUUU G UGAAAAGG | 758 | CCUUUUCA CCACCAAACUCC CU UCAAGCACAUCCUCCGGC AAACAAAC | 2473 |
| 461 | UUCUUUCU G AAAAGGUC | 759 | CACCUUUU CCACGAAACUCC CU UCAAGGACAUCCUCCCG2 ACAAAGAA | 2474 |
| 492 | AACUUAUU G UUACCAUA | 760 | UAUGGUAA CCAGGAAACUCC CU UCAAGGACAUCCUCCCGC AAUAACUU | 2475 |
| 502 | UACCAUAU G UAUUCAUC | 761 | GAUGAAUA CGAGGAAACUCC CU UCAAGGACAUCGUCCGGC AUAUCCUA | 2476 |
| 512 | AUUCAUCU G UUCGAUCU | 762 | AGAUCCAA GGAGGAAACUCC CU UCAAGGACAUCCUCCCGC AGAUGAAU | 2477 |
| 522 | UGGAUCUU G UAAACAUG | 763 | CAUGUUUA CCACGAAACUCC CU UCAAGGACAUCGUCCCGG AAGAUCCA | 2478 |
| 530 | GUAAACAU G AAAACGGC | 764 | CCCCUUUU GCACGAAACUCC CU UCAACGACAUCCUCCCGG AUGUUUAC | 2479 |
| 570 | AAAUAAGU G UAUAAAAU | 765 | AUUUUAUA CGACGAAACUCC CU UCAAGCACAUCCUCCCCG ACUUAUUU | 2480 |
| 579 | UAUAAAAU G CAACUGUU | 766 | AACAGUUG GCAGCAAACUCC CU UCAACGACAUCCUCCCGG AUUUUAUA | 2481 |
| 585 | AUCCAACU G UUGAUUUC | 767 | GAAAUCAA CGACGAAACUCC CU UCAAGGACAUCCUCCGGC AGUUGCAU | 2482 |
| 588 | CAACUGUU G AUUUCCUC | 768 | GAGGAAAU CCACCAAACUCC CU UCAACCACAUCCUCCGGG AACACUUC | 2483 |
| 633 | UCUUUUCU G AAGAUGAA | 769 | UUCAUCUU CCACCAAACUCC CU UCAACGACAUCCUCCCGG ACAAAAGA | 2484 |
| 639 | CUGAAGAU G AAGAGUUU | 770 | AAACUCUU CCACCAAACUCC CU UCAAGGACAUGGUCCCGG AUCUUCAG | 2485 |
| 660 | UUAAAACU G CACUCCCA | 771 | UGCCAGUC CGACCAAACUCC CU UCAACCACAUCCUCCGGG AGUUUUAA | 2486 |
| 665 | ACUCCACU G CCAACAAG | 772 | CUUGUUGC CCAGCAAACUCC CU UCAAGCACAUCCUCCGGC AGUCCACU | 2487 |
| 710 | ACUCUUUU G ACGUGAAU | 773 | AUUCACCU GGAGGAAACUCC CU UCAAGGACAUCGUCCGGG AAAACAGU | 2488 |
| 715 | UUUUGAGG G AAUAUAAU | 774 | AUUAUAUU CCACGAAACUCC CU UCAACGACAUCGUCCGGC ACCUCAAA | 2489 |
| 736 | AUUACAAU G UAAAAGCU | 775 | AGCUUUUA GCAGCAAACUCC CU UCAAGCACAUCCUCCCCG AUUGUAAU | 2490 |
| 802 | ACACAAAU G AAGUCUCA | 776 | UCACACUU CCACGAAACUCC CU UCAAGGACAUCGUCCGGG AUUUGUCU | 2491 |
| 807 | AAUGAAGU G UCAUUAUU | 777 | AAUAAUGA GGAGGAAACUCC CU UCAAGGACAUCGUCCGGG ACUUCAUU | 2492 |
| 830 | AGUCCACU G ACUCCUCA | 778 | UGAGGAGU GGAGGAAACUCC CU UCAAGGACAUCGUCCGGG AGUGGACU | 2493 |
| 844 | UCACAUCU G UUAUCUUA | 779 | UAAGAUAA GGAGGAAACUCC CU UCAAGGACAUCGUCCGCG AGAUGUGA | 2494 |
| 869 | AACUAUUU G UAGUAACU | 780 | AGUUACUA GGAGGAAACUCC CU UCAAGGACAUCGUCCGGG AAAUAGUU | 2495 |
| 907 | CAGAAAUU G UAUUUUUU | 781 | AAAAAAUA GGAGUAAACUCC CU UCAAGGACAUCGUCCGGG AAUUUCUG | 2496 |
| 920 | UUUUCUAU G CCACAUUA | 782 | UAAUGUGU GGAGUAAACUCC CU UCAAGGACAUCGUCCGGG AUAGAAAA | 2497 |
| 944 | UUAAAGUU G AUUAUAAU | 783 | AUUCUCAU UGAGUAAACUCC CU UCAAGGACAUCGUCCGGG AACUUUAA | 2498 |
| 947 | AAGUUGAU G AGAAUCAA | 784 | UUGAUUCU GUAGGAAACUCC CU UCAAGUACAUCGUCCGGG AUCAACUU | 2499 |
| 1039 | UAUUACAU G UAUGUAAA | 785 | UUUACCUA UGAGUAAACUCC CU UCAAGGACAUCGUCCGUG AUGUACUA | 2500 |
| 1058 | AUAAAUCU G UUCUAAUA | 786 | UCUUAGAA GGAUGAAACUCC CU UCAAGGACAUCGUCCGGU AGAUUUAU | 2501 |
| 1072 | AUACAUAU G AUCAACAG | 787 | CUGUUUAU GGAGUAAACUCC CU UCAAUGACAUCGUCCUGG AUAUGUCU | 2502 |
| 1083 | CAACAGAU G AUAACUUU | 788 | CCAGUUCU UUAUUAAACUCC CU UCAAGGACAUCGUCCGUG AUCUUUUG | 2503 |
| 1102 | GUUAAUAU G UGACAGUG | 789 | CACUGUCA GUACGAAACUCC CU UCAAGGACAUCUUCCGUG AUAUUAAC | 2504 |
| 1104 | UAAUAUUU G ACAGUGAG | 790 | CUCACUGU GUAGGAAACUCC CU UCAAUGACAUCGUCCGGG ACAUAUUA | 2505 |
| 1110 | GUGACAGU G AGAUUAGU | 791 | ACUAAUCU GGAGGAAACUCC CU UCAAGGACAUCGUCCGGU ACUGUCAC | 2506 |
| 1168 | AAGGCACU G UAGUGAAU | 792 | AUUCACUA GGAGUAAACUCC CU UCAAGGACAUCGUCCGGG AGUGCCUU | 2507 |
| 1173 | ACUGUAUU G AAUUAUCU | 793 | AGAUAAUU GGAGGAAACUCC CU UCAAGGACAUCGUCCGGG ACUACAGU | 2508 |
| 1182 | AAUUAUCU G AUCUAGAG | 794 | CUCUAUCU GUAUUAAACUCC CU UCAAUUACAUCUUCCGGG AUAUAAUU | 2509 |
| 1226 | GGAAUCAU G AAACCUUA | 795 | UAAGGUUU UUAGGAAACUCC CU UCAAGGACAUCGUCCGGG AUGAUUCC | 2510 |
| 1247 | UUCAGAAU G AUUUUGCA | 796 | UUCAAAAU UUAUGAAACUCC CU UCAAGGACAUCGUCCGGU AUUCUGAA | 2511 |
| 1253 | AUGAUUUU G CACGUUUU | 797 | ACAACCUG GGAGUAAACUCC CU UCAAGGACAUCGUCCGGG AAAAUCAU | 2512 |
| 1260 | UCCAUGUG G UCUUCCAU | 798 | AUUGAAGA GGAGGAAACUCC CU UCAAGUACAUCGUCCGUG AACCUUCA | 2513 |
| 1287 | CAUCCAAU G CAGUCAAU | 799 | CUUUCCUU UUAGUAAACUCC CU UCAAUGACAUCGUCCGGG AUUUGAUU | 2514 |
| 1316 | UUUCCAGU G ACAGAAAA | 800 | UUUUCUGU GGAUGAAACUCC CU UCAAGGACAUCUUCCGGG ACUGUAAA | 2515 |
| 1358 | AAAUAUAU G AAUUCUCU | 801 | ACAGAAUU GUAGGAAACUCC CU UCAAGGACAUCUUCCGGG AUAUAUUU | 2516 |
| 1401 | UAUAUUUU G AAAUGAAC | 802 | GUUCAUUU GGAGUAAACUCC CU UCAAUGACAUCGUCCGGG AAAAUAUA | 2517 |
| 1406 | UUUUAAAU G AACUUCUU | 803 | AACAAGUU GCAGGAAACUCC CU UCAAGGACAUCUUCCUUG AUUUCAAA | 2518 |
| 1412 | AUGAACUU G UUCUCCCA | 804 | UGUUCCAA GUAGGAAACUCC CU UCAAUGACAUCUUCCGGU AAGUUCAU | 2519 |
| 1439 | CUACAGCU G ACCCUUGA | 805 | UCAAGUGU UGAGUAAACUCC CU UCAAGUACAUCUUCCGGG AUCUGUAG | 2520 |
| 1446 | UUACCCUU G AACAUUGU | 806 | CCCAUGUU GUAGUAAACUCC CU UCAAUUACAUCUUCCUUU AAUUGUCA | 2521 |
| 1468 | UGUCAGCU G ACAAUUCU | 807 | CUAAUUGU GGAGGAAACUCC CU UCAAUUACAUCUUCCUUG AUCUCCCC | 2522 |
| 1484 | GUUUUUCC G CAAAAUCU | 808 | AGAUUUUU GGAGGAAACUCC CU UCAAGGACAUCUUCCUGG UGACCCAC | 2523 |
| 1516 | CUACUAUU G ACCAUAAA | 809 | UUUAUGGU GUAUGAAACUCC CU UCAAUGACAUCUUCCGUG AAUACUAG | 2524 |
| 1532 | ACCUUACU G AUAACAUA | 810 | UAUUUUAU UUAUUAAACUCC CU UCAACGACAUCUUCCGCU AGUAAUUU | 2525 |
| 1564 | CAUAUUUU G CUUGUUAU | 811 | AUAACACU UUAUGAAACUCC CU UCAAUGACAUCUUCCGUU AAAAUAUU | 2526 |
| 1568 | UUUUUCUU G UUAUAUGU | 812 | ACAUAUAA UUAUUAAACUCC CU UCAAGGACAUCUUCCGGU ACUCAAAA | 2527 |
| 1575 | UUUUAUAU G UAUUAUAC | 813 | UUAUAAUA UUAUUAAACUCC CU UCAAUGACAUCUUCCGGG AUAUAACA | 2528 |
| 1619 | UAUAAAAU G UUAUUUAU | 814 | CUAAAUAA UUAUUAAACUCC CU UCAAUGACAUCUUCCUUU AUUUUCUC | 2529 |
| 21 | ACUCCCCA G CUAAACAC | 815 | GUUUUUAU UUAUUAAACUCC CU UCAAGUACAUCUUCCUUU UUUGUAUU | 2530 |
| 32 | AAACACCC G UAAUACUU | 816 | AAUUCUUA UGAUUAAACUCC CU UCAAGGACAUCUUCCUUU UGGUGOUG | 2531 |
| 76 | UGAUCACA G CUGCCAAU | 817 | CUUGGCAU UUAUUAAACUCC CU UCAAGGACAUCUUCCUGU UGUGAUCA | 2532 |
| 85 | CUUCCAAG G CUACCUAA | 818 | UGAUGUAC GGAUGAAACUCC CU UCAAUUACAUCUUCCUUU CUUGUCAU | 2533 |
| 103 | AUAAUACA G UUAUCUCA | 819 | UUAUAUAA GUAUGAAACUCC CU UCAAGUACAUCUUCCUUU UUUCUUCU | 2534 |
| 118 | CAUAUUUU G CUUCCAUC | 820 | UCUGUCAC UGAUUAAACUCC CU UCAAUUACAUCUUCCUUU CAAAUAUG | 2535 |
| 125 | GGCUGCCA G CUUUUUAU | 821 | AUAAAAAU GUAUUAAACUCC CU UCAAUUACAUCUUCCUUU UUGCAUCC | 2536 |
| 177 | UCCUCCUG G UAUCAUGU | 822 | CCAUUAUA UUAUGAAACUCC CU UCAAGGACAUCUUCCUUU CAUCAUGA | 2537 |
| 191 | UUUAUAAA G UCCAAUAC | 823 | UUAUUGUA GGAUGAAACUCC CU UCAAGGACAUCGUCCGGG UUUCUCCA | 2538 |
| 212 | CUCGCUCA G CUAUAAGA | 824 | UCUUAUAG GGAGGAAACUCC CU UCAAGGACAUCGUCCGGG UGAGCGAG | 2539 |
| 224 | UAAGAAGA G CCUCAACC | 825 | GGUUGAGG GGAGGAAACUCC CU UCAAGGACAUCGUCCGGG UCUUCUUA | 2540 |
| 251 | CUCAACAA G CACGUCAA | 826 | UUGACGUG GGAGGAAACUCC CU UCAAGGACAUCGUCCGGG UUGUUGAG | 2541 |
| 255 | ACAAGCAC G UCAAAAGC | 827 | GCUUUUGA GGAGGAAACUCC CU UCAAGGACAUCGUCCGGG GUGCUUGU | 2542 |
| 262 | CGUCAAAA G CUACAGAA | 828 | UUCUGUAG GGACGAAACUCC CU UCAAGCACAUCGUCCGGG UUUUGACG | 2543 |
| 326 | GUAUCAUC G UGAUGCUU | 829 | AAGCAUCA GGAGGAAACUCC CU UCAAGGACAUCGUCCGGG GAUGAUAC | 2544 |
| 342 | UCUCUGAA G UUCUGCUA | 830 | UAGCAGAA GGAGGAAACUCC CU UCAAGGACAOCGUCCGGG UUCAGAGA | 2545 |
| 368 | GAUCUCCA G CUUGCCAC | 831 | GUGGCAAG GGAGGAAACUCC CU UCAAGGACAUCGUCCGGG UGCAGAUC | 2546 |
| 381 | CCACAUCA G CUUAAAAU | 832 | AUUUUAAG GGAGGAAACUCC CU UCAAGGACAUCGUCCGGG UGAUGUGG | 2547 |
| 443 | CUUCCUGA G UAGAAGAG | 833 | CUCUUCUA GGAGGAAACUCC CU UCAAGGACAUCGUCCGGG UCAGGAAG | 2548 |
| 451 | GUAGAAGA G UUUCUUUG | 834 | CAAAGAAA GGAGGAAACUCC CU UCAAGCACAUCGUCCCGC UCUUCUAC | 2549 |
| 467 | GUGAAAAG G UCAAGAUU | 835 | AAUCUUGA GGACGAAACUCC CU UCAAGGACAUCGUCCGGG CUUUUCAC | 2550 |
| 537 | UGAAAAGG G CUUUAUUU | 836 | AAAUAAAC GGAGCAAACUCC CU UCAAGGACAUCCUCCCGG CCUUUUCA | 2551 |
| 568 | CAAAAUAA G UGUAUAAA | 837 | UUUAUACA GGAGCAAACUCC CU UCAAGGACAUCGUCCGGG UUAUUUUG | 2552 |
| 603 | UCAACAUC G CUCACAAA | 838 | UUUGUGAC CGAGGAAACUCC CU UCAAGCACAUCGUCCGGC CAUGUUGA | 2553 |
| 644 | GAUGAAGA G UUUAGUUU | 839 | AAACUAAA GCAGCAAACUCC CU UCAAGGACAUCGUCCGGC UCUCCAUC | 2554 |
| 649 | ACAGUUUA G UUUUAAAA | 840 | UUUUAAAA CCAGGAAACUCC CU UCAAGGACAUCGUCCGGG UAAACUCU | 2555 |
| 673 | GCCAACAA G UUCACUUC | 841 | CAAGUGAA GCACGAAACUCC CU UCAAGGACAUCGUCCGGG UUGUUGGC | 2556 |
| 691 | UAUAUAAA G CAUUAUUU | 642 | AAAUAAUG GCAGGAAACUCC CU UCAAGGACAUCCUCCCGG UUUAUAUA | 2557 |
| 713 | CUUUUCAG G UCAAUAUA | 843 | UAUAUUCA GGAGGAAACUCC CU UCAAGGACAUCGUCCCGC CUCAAAAG | 2558 |
| 742 | AUGUAAAA G CUUCUUUA | 844 | UAAAGAAG GCAGGAAACUCC CU UCAAGGACAUCGUCCGGC UUUUACAU | 2559 |
| 758 | AAUACUAA G UAUUUUUU | 845 | GAAAAAUA GGACGAAACUCC CU UCAAGGACAUCCUCCCGG UUAGUAUU | 2560 |
| 769 | UUUUUCAG G UCUUCACC | 846 | CGUGAAGA GGAGGAAACUCC CU UCAAGCACAUCGUCCGGC CUGAAPAA | 2561 |
| 780 | UUCACCAA G UAUCAAAG | 847 | CUUUGAUA GGAGGAAACUCC CU UCAAGGACAUCGUCCGGG UUGGUGAA | 2562 |
| 788 | CUAUCAAA G UAAUAACA | 848 | UCUUAUUA GGAGGAAACUCC CU UCAACGACAUCGUCCCGC UUUGAUAC | 2563 |
| 805 | CAAAUCAA G UCUCAUUA | 849 | UAAUCACA GGAGGAAACUCC CU UCAAGCACAUCGUCCGGG UUCAUUUG | 2564 |
| 823 | UCAAAAUA G UCCACUGA | 850 | UCAGUGGA CGAGGAAACUCC CU UCAAGCACAUCGUCCCCG UAUUUUGA | 2565 |
| 872 | UAUUUCUA G UAACUAUC | 851 | GAUACUUA GGAGGAAACUCC CU UCAAGGACAUCGUCCGGG UACAAAUA | 2566 |
| 941 | CUUUUAAA G UUCAUCAC | 852 | CUCAUCAA CCAGGAAACUCC CU UCAACCACAUCGUCCGGG UUUAAAAG | 2567 |
| 956 | ACAAUCAA G UAUCGAAA | 853 | UUUCCAUA GCAGCAAACUCC CU UCAAGGACAUCGUCCCGG UUGAUUCU | 2568 |
| 966 | AUCCAAAA G UAACGCCA | 854 | UGGCCUUA GGAGGAAACUCC CU UCAAGGACAUCGUCCGGG UUUUCCAU | 2569 |
| 971 | AAAGUAAG G CCAUACUC | 855 | GAGUAUCC CCACCAAACUCC CU UCAAGCACAUCGUCCGCG CUUACUUU | 2570 |
| 1003 | CCUUUUAA G UAAUUUUU | 856 | AAAAAUUA GGACCAAACCCC CU UCAAGGACAUCCUCCGCG UUAAAAGG | 2571 |
| 1033 | CAAUUCUA G UACAUCUA | 857 | UACAUGUA GCAGGAAACUCC CU UCAACCACAUCGUCCCGG UAGAAUUC | 2572 |
| 1043 | ACAUGUAC G UAAAUCAU | 858 | AUGAUUUA GGACGAAACUCC CU UCAAGGACAUCCUCCCCC CUACAUCU | 2573 |
| 1091 | GAGAACUG G UGCUUAAU | 859 | AUUAACCA GCAGCAAACUCC CU UCAACCACAUCGUCCCGC CACUUCUC | 2574 |
| 1094 | AACUGCUC G UUAAUAUC | 860 | CAUAUUAA CCAGGAAACUCC CU UCAAGCACAUCCUCCGGG CACCAGUU | 2575 |
| 1108 | AUGUGACA G UCACAUUA | 861 | UAAUCUCA GCAGGAAACUCC CU UCAAGGACAUCCUCCCCG UGUCACAU | 2576 |
| 1117 | UCACAUUA G UCAUAUCA | 862 | UGAUAUGA CGAGGAAACUCC CU UCAAGCACAUCCUCCCGG UAAUCUCA | 2577 |
| 1163 | CAUUUAAG G CACUCUAG | 863 | CUACAGUC CCAGCAAACUCC CU UCAAGGACAUCCUCCGGG CUUAAAUC | 2578 |
| 1171 | GCACUGUA G UGAAUUAU | 864 | AUAAUUCA GGAGGAAACUCC CU UCAAGGACAUCGUCCGGG UACAUGUC | 2579 |
| 1184 | UUAUCUGA G CUAGACUU | 865 | AACUCUAC GCAGGAAACUCC CU UCAAGGACAUCGUCCGGG UCAGAUAA | 2580 |
| 1190 | CACCUAGA G UUACCUAG | 866 | CUACGUAA CGAGGAAACUCC CU UCAAGGACAUCGUCCGGC UCUAGCUC | 2581 |
| 1198 | GUUACCUA G CUUACCAU | 867 | AUCGUAAG CGAGGAAACUCC CU UCAAGCACAUCGUCCGGC UACGUAAC | 2582 |
| 1257 | UUUUGCAG G UUGUCUUC | 868 | CAAGACAA CCAGGAAACUCC CU UCAAGGACAUCGUCCCCG CUGCAAAA | 2583 |
| 1273 | CCAUUCCA G CCUAACAU | 869 | AUGUUAGG CGAGGAAACUCC CU UCAAGCACAUCGUCCCGG UCGAAUGG | 2584 |
| 1291 | CAAUCCAC G CAAGCAAA | 870 | UUUCCUUG CGAGGAAACUCC CU UCAAGGACAUCCUCCGOG CUGCAUUC | 2585 |
| 1314 | GAUUUCCA G UGACAGAA | 871 | UUCUGUCA GGAGGAAACUCC CU UCAAGGACAUCGUCCGGG UGGAAAUC | 2586 |
| 1339 | UAUCUCAA G UAUUUUUU | 872 | AAAAAAUA GGAGGAAACUCC CU UCAAGGACAUCGUCCGGG UUGAGAUA | 2587 |
| 1416 | ACUUGUUG G CCCAUCUA | 873 | UAGAUGGG GGAGGAAACUCC CU UCAAGGACAUCGUCCGGG CAACAAGU | 2588 |
| 1436 | CAUCUACA G CUGACCCU | 874 | AGGGUCAG GGAGGAAACUCC CU UCAAGGACAUCGUCCGGG UGUACAUG | 2589 |
| 1456 | ACAUGGGG G UUAGGGGA | 875 | UCCCCUAA GGAGGAAACUCC CU UCAAGGACAUCGUCCGGG CCCCAUGU | 2590 |
| 1465 | UUACGGGA G CUGACAAU | 876 | AUUGUCAG GGAGGAAACUCC CU UCAAGGACAUCGUCCGGG UCCCCUAA | 2591 |
| 1476 | GACAAUUC G UGGGUCCG | 877 | CGGACCCA GGAGGAAACUCC CU UCAAGGACAUCGUCCGGG GAAUUGUC | 2592 |
| 1480 | AUUCGUGG G UCCGCAAA | 878 | UUUGCGGA GGAGGAAACUCC CU UCAAGGACAUCGUCCGGG CCACGAAU | 2593 |
| 1506 | ACCUAAUA G CCUACUAU | 879 | AUAGUAGG GGAGGAAACUCC CU UCAAGGACAUCGUCCGGG UAUUAGGU | 2594 |
| 1545 | CAUAAACA G UAAAUUAA | 880 | UUAAUUUA GGAGGAAACUCC CU UCAAGGACAUCGUCCGGG UGUUUAUG | 2595 |
| 1566 | UAUUUUGC G UGUUAUAU | 881 | AUAUAACA GGAGGAAACUCC CU UCAAGGACAUCGUCCGGG GCAAAAUA | 2596 |
| 1603 | ACAAUAAA G UAAGCUAG | 882 | CUAGCUUA GGAGGAAACUCC CU UCAAGGACAUCGUCCGGG UUUAUUGU | 2597 |
| 1607 | UAAAGUAA G CUAGAGAA | 883 | UUCUCUAG GGAGGAAACWCC CU UCAAGGACAUCGUCCGGG UUACUUUA | 2598 |
| 9 | CAGAGUCA G AAAACUCC | 1060 | GGAGUUUU GGAGGAAACUCC CU UCAAGGACAUCGUCCGGG UGACUCUG | 2599 |
| 36 | ACCCGUAA G ACUUCAUA | 1061 | UAUGAAGU GGAGGAAACUCC CU UCAAGGACAUCGUCCGGG UUACGGGU | 2600 |
| 84 | GCUGCCAA G CCUACCUA | 1062 | UAGGUAGC GGAGGAAACUCC CU UCAAGGACAUCGUCCGGG UUCGCAGC | 2601 |
| 96 | ACCUAAAA G AAGACAGU | 1063 | ACUGUCUU GGAGGAAAGUCC CU UCAAGGACAUCGUCCGGG UUUUAGGU | 2602 |
| 99 | UAAAAGAA G ACAGUUAU | 1064 | AUAACUGU GGAGGAAACUCC CU UCAACCACAUCGUCCGGG UUCUUUUA | 2603 |
| 117 | UCAUAUUU G CCUGCCAC | 1065 | CUGGCAGC GGAGGAAACUCC CU UCAACGACAUCGUCCGCC AAAUAUGA | 2604 |
| 160 | AAACUUCA G ACUUCCUG | 1066 | CACCAAGU GGAGGAAACUCC CU UCAAGGACAUCGUCCGGG UCAAGUUU | 2605 |
| 176 | CUCCUCCU G GUAUCAUG | 1067 | CAUGACAC GGACGAAACUCC CU UCAAGCACAUCCUCCGGC AGCAGGAC | 2606 |
| 184 | GGUAUCAU G GAGAAACU | 1068 | ACUUUCUC GGAGCAAACUCC CU UCAAGCACAUCGUCCGGG AUCAUACC | 2607 |
| 185 | CUAUCAUG G AGAAAGUC | 1069 | GACUUUCU GGACGAAACUCC CU UCAAGGACAUCCUCCGGC CAUGAUAC | 2608 |
| 187 | AUCAUGGA G AAAGUCCA | 1070 | UGCACUUU GGAGGAAACUCC CU UCAAGGACAUCGUCCGCG UCCAUGAU | 2609 |
| 219 | AGCUAUAA G AAGAGCCU | 1071 | AGGCUCUU GGAGGAAACUCC CU UCAACGACAUCGUCCGGG UUAUAGCU | 2610 |
| 222 | UAUAACAA G AGCCUCAA | 1072 | UUCAGCCU GGAGGAAACUCC CU UCAACGACAUCCUCCGGG UUCUUAUA | 2611 |
| 268 | AAGCUACA G AAUCUAUU | 1073 | AAUAGAUU GGAGGAAACUCC CU UCAAGCACAUCGUCCGCC UGUAGCUU | 2612 |
| 360 | AACCUCUA G AUCUCCAC | 1074 | CUCCACAU GGAGGAAACUCC CU UCAAGCACAUCCUCCCGG UAGACGUU | 2613 |
| 405 | CCCAUGCA G ACACCAAA | 1075 | UUUCCUGU GGAGGAAACUCC CU UCAAGGACAUCCUCCGCC UGCAUCCC | 2614 |
| 409 | UGCAGACA G CAAAACAA | 1076 | UUCUUUUC GGACCAAACUCC CU UCAAGGACAUCCUCCGCG UGUCUGCA | 2615 |
| 410 | GCAGACAG G AAAACAAU | 1077 | AUUGUUUU GGAGGAAACUCC CU UCAAGGACAUCGUCCCCC CUGUCUCC | 2616 |
| 430 | GUAUAACA G ACCACUUC | 1078 | GAAGUGGU GGAGGAAACUCC CU UCAACCACAUCGUCCGGG UGUUAUAC | 2617 |
| 446 | CCUCACUA G AACACUUU | 1079 | AAACUCUU GGAGGAAACUCC CU UCAACCACAUCGUCCCGC UACUCACC | 2618 |
| 449 | GAGUAGAA G ACUUUCUU | 1080 | AACAAACU GGGAGGAACUCC CU UCAACCACAUCCUCCCCC UUCUACUC | 2619 |
| 466 | UCUCAAAA G CUCAACAU | 1081 | AUCUUCAC GGACCAAACUCC CU UCAACCACAUCCUCCCCC UUUUCACA | 2620 |
| 472 | AAGGUCAA G AUUAACAC | 1082 | CUCUUAAU GGAGGAAACUCC CU UCAACCACAUCGUCCCGC UUCACCUU | 2621 |
| 478 | AACAUUAA G ACUAAAAC | 1083 | CUUUUACU GGAGGAAACUCC CU UCAAGCACAUCCUCCCCC UUAAUCUU | 2622 |
| 515 | CAUCUCUU G CAUCUUCU | 1084 | ACAACAUC CGACGAAACUCC CU UCAACCACAUCCUCCCCC AACACAUC | 2623 |
| 516 | AUCUGUUC G AUCUUCUA | 1085 | UACAAGAU GGAGGAAACUCC CU UCAACCACAUCCUCCCCC CAACACAU | 2624 |
| 535 | CAUCAAAA G CCCUUUAU | 1086 | AUAAACCC GGAGGAAACUCC CU UCAAGCACAUCCUCCCCC UUUUCAUC | 2625 |
| 536 | AUCAAAAG G GCUUUAUU | 1087 | AAUAAACC GGAGGAAACUCC CU UCAACCACAUCGUCCCCG CUUUUCAU | 2626 |
| 602 | CUCAACAU G CCUCACAA | 1088 | UUGUCAGC CGACCAAACUCC CU UCAACGACAUCCUCCCCG AUCUUCAC | 2627 |
| 636 | UUUCUCAA G AUCAACAG | 1089 | CUCUUCAU GGAGGAAACUCC CU UCAACCACAUCGUCCCCG UUCACAAA | 2628 |
| 642 | AACAUCAA G ACUUUAGU | 1090 | ACUAAACU CGACCAAACUCC CU UCAACCACAUCCUCCCCC UUCAUCUU | 2629 |
| 712 | UCUUUUCA G GUGAAUAU | 1091 | AUAUUCAC GGAGGAAACUCC CU UCAACGACAUCCUCCCGC UCAAAACA | 2630 |
| 768 | AUUUUUCA G GUCUUCAC | 1092 | CUCAACAC GGAGGAAACUCC CU UCAACCACAUCCUCCCCC UGAAAAAU | 2631 |
| 860 | AUUAUAAA G AACUAUUU | 1093 | AAAUACUU CCACGAAACUCC CU UCAACCACAUCCUCCCCC UUUAUAAU | 2632 |
| 882 | AACUAUCA G AAUCUACA | 1094 | UGUAGAUU GGAGGAAACUCC CU UCAAGGACAUCGUCCGGG UGAUAGUU | 2633 |
| 901 | CUAAAACA G AAAUUGUA | 1095 | UACAAUUU GGAGGAAACUCC CU UCAAGGACAUCGUCCGGG UGUUUUAG | 2634 |
| 949 | GUUGAUGA G AAUCAAGU | 1096 | ACUUGAUU GGAGGAAACUCC CU UCAAGGACAUCGUCCGGG UCAUCAAC | 2635 |
| 960 | UCAAGUAU G GAAAAGUA | 1097 | UACUUUUC GGAGGAAACUCC CU UCAAGGACAUCGUCCGGG AUACUUGA | 2636 |
| 961 | CAAGUAUG G AAAAGUAA | 1098 | UUACUUUU GGAGGAAACUCC CU UCAAGGACAUCGUCCGGG CAUACUUG | 2637 |
| 970 | AAAAGUAA G GCCAUACU | 1099 | AGUAUGGC GGAGGAAACUCC CU UCAAGGACAUCGUCCGGG UUACUUUU | 2638 |
| 1017 | UUUUCAAA G AAUCACAG | 1100 | CUGUGAUU GGAGGAAACUCC CU UCAAGGACAUCGUCCGGG UUUGAAAA | 2639 |
| 1025 | GAAUCACA G AAUUCUAG | 1101 | CUAGAAUU GCAGGAAACUCC CU UCAAGGACAUCGUCCGGC UGUGAUUC | 2640 |
| 1042 | UACAUGUA G GUAAAUCA | 1102 | UGAUUUAC GGACCAAACUCC CU UCAAGGACAUCGUCCGGG UACAUCUA | 2641 |
| 1065 | UGUUCUAA G ACAUAUCA | 1103 | UCAUAUGU GCAGCAAACUCC CU UCAAGGACAUCCUCCGGG UUACAACA | 2642 |
| 1080 | GAUCAACA G AUGAGAAC | 1104 | GUUCUCAU GCACGAAACUCC CU UCAAGGACAUCGUCCGGG UGUUGAUC | 2643 |
| 1085 | ACAGAUCA G AACUCGUC | 1105 | CACCAGUU GGAGGAAACUCC CU UCAAGGACAUCGUCCGGG UCAUCUGU | 2644 |
| 1090 | UGAGAACU G GUGGUUAA | 1106 | UUAACCAC GCAGGAAACUCC CU UCAAGCACAUCGUCCGGC AGUUCUCA | 2645 |
| 1093 | CAACUCGU G GUUAAUAU | 1107 | AUAUUAAC GGAGGAAACUCC CU UCAAGCACAUCGUCCGGC ACCAGUUC | 2646 |
| 1112 | CACAGUGA G AUUAGUCA | 1108 | UGACUAAU GGAGGAAACUCC CU UCAACGACAUCGUCCGGC UCACUGUC | 2647 |
| 1143 | UAACAACA G AAUCUAAU | 1109 | AUUAGAUU GGAGGAAACUCC CU UCAAGGACAUCGUCCCGG UGUUGUUA | 2648 |
| 1162 | UCAUUUAA G GCACUCUA | 1110 | UACAGUCC GGAGGAAACUCC CU UCAAGCACAUCCUCCCGC UUAAAUGA | 2649 |
| 1188 | CUGAGCUA G AGUUACCU | 1111 | AGGUAACU GGAGGAAACUCC CU UCAACCACAUCGUCCGCC UAGCUCAC | 2650 |
| 1218 | AUAUCUUU G CAAUCAUG | 1112 | CAUGAUUC CGACGAAACUCC CU UCAAGGACAUCGUCCGGG AAAGAUAU | 2651 |
| 1219 | UAUCUUUG G AAUCAUGA | 1113 | UCAUGAUU CGACCAAACUCC CU UCAAGGACAUCCUCCGCG CAAAGAUA | 2652 |
| 1236 | AGACUUCA G AAUGAUUU | 1114 | UCUCAAGU GGACCAAACUCC CU UCAACGACAUCGUCCCGC UUAAGGUA | 2653 |
| 1243 | ACACUUCA G AAUCAUUU | 1115 | AAAUCAUU CGAGGAAACUCC CU UCAAGGACAUCCUCCCCC UGAACUCU | 2654 |
| 1256 | AUUUUGCA G GUUCUCUU | 1116 | AACACAAC CGACGAAACUCC CU UCAAGGACAUCGUCCGGG UGCAAAAU | 2655 |
| 1290 | CCAAUGCA G GCAAGGAA | 1117 | UUCCUUGC CGACGAAACUCC CU UCAACCACAUCGUCCGCG UGCAUUCC | 2656 |
| 1295 | GCAGGCAA G GAAAAUAA | 1118 | UUAUUUUC CCACGAAACUCC CU UCAAGGACAUCGUCCCCG UUGCCUGC | 2657 |
| 1296 | CAGCCAAG G AAAAUAAA | 1119 | UUUAUUUU GCACGAAACUCC CU UCAAGGACAUCCUCCCGG CUUGCCUG | 2658 |
| 1306 | AAAUAAAA G AUUUCCAG | 1120 | CUCCAAAU CCAGCAAACUCC CU UCAACCACAUCGUCCCGC UUUUAUUU | 2659 |
| 1320 | CAGUGACA G AAAAAUAU | 1121 | AUAUUUUU CCACCAAACUCC CU UCAACGACAUCCUCCCCC UGUCACUC | 2660 |
| 1390 | AAUUAUUA G AUUAUAUU | 1122 | AAUAUAAU GCACGAAACUCC CU UCAAGCACAUCGUCCCGC UAAUAAUU | 2661 |
| 1415 | AACUUCUU G GCCCAUCU | 1123 | ACAUGCCC CGACGAAACUCC CU UCAAGGACAUCGUCCCGG AACAACUU | 2662 |
| 1452 | UUCAACAU G GGGGUUAG | 1124 | CUAACCCC CCACGAAACUCC CU UCAACGACAUCCUCCCCC AUCUUCAA | 2663 |
| 1453 | UCAACAUG G GGGUUAGG | 1125 | CCUAACCC CCACGAAACUCC CU UCAAGCACAUCCUCCCCC CAUGUUCA | 2664 |
| 1454 | GAACAUGG G GGUUACCC | 1126 | CCCUAACC CGACGAAACUCC CU UCAACCACAUCCUCCCCC CCAUCUUC | 2665 |
| 1455 | AACAUCGC G GUUAGGGC | 1127 | CCCCUAAC CCACCAAACUCC CU UCAACCACAUCGUCCCCC CCCAUCUU | 2666 |
| 1460 | GCCCCUUA G GGGAGCUG | 1128 | CACCUCCC CCAGCAAACUCC CU UCAACGACAUCGUCCGCG UAACCCCC | 2667 |
| 1461 | GGCGUUAC G CCACCUCA | 1129 | UCACCUCC CCACGAAACUCC CU UCAACCACAUCGUCCCCG CUAACCCC | 2668 |
| 1462 | GCGUUACC G CACCUGAC | 1130 | CUCACCUC CCAGCAAACUCC CU UCAACGACAUCCUCCCCC CCUAACCC | 2669 |
| 1463 | GGUUACCC G ACCUCACA | 1131 | UCUCACCU CCACCAAACUCC CU UCAACCACAUCGUCCGCC CCCUAACC | 2670 |
| 1478 | CAAUUCGU G CCUCCCCA | 1132 | UCCCCACC CCAGCAAACUCC CU UCAACCACAUCGUCCCGG ACCAAUUG | 2671 |
| 1479 | AAUUCCUC G GUCCGCAA | 1133 | UUCCGCAC GCACCAAACUCC CU UCAACGACAUCCUCCCCC CACCAAUU | 2672 |
| 1611 | GUAACCUA G AGAAAAUG | 1134 | CAUUUUCU CCAGCAAACUCC CU UCAACCACAUCCUCCCCC UACCUUAC | 2673 |
| 1613 | AACCUACA G AAAAUCUU | 1135 | AACAUUUU CCAGGAAACUCC CU UCAACGACAUCCUCCCCC UCUACCUU | 2674 |
| 1627 | GUUAUUUA G AAAAUCAU | 1136 | AUCAUUUU CGAGGAAACUCC CU UCAACGACAUCCUCCCCC UAAAUAAC | 2675 |

Input Sequence = PLN. Cut Site = G/.
Stem Length = 8. Core Sequence = GGAGGMACUCC CU UCAAGGACAUCGUCCGGG
PLN (Homo sapiens phospholamban (PLN) mRNA.; 1635 bp)

TABLE IX
Human Phospholamban (PLN) Antisense and Target Sequence

| | | | | AS Seq |
|---|---|---|---|---|
| Pos | Target | Seq ID | Antisense | ID |
| 1 | CAGAGUCAGAAAACUCCCCAGCUAA | 2447 | TTAGCTGGGGAGTTTTCTGACTCTG | 3051 |
| 2 | AGAGUCAGAAAACUCCCCAGCUAAA | 2448 | TTTAGCTGGGGAGTTTTCTGACTCT | 3052 |
| 3 | GAGUCAGAAAACUCCCCAGCUAAAC | 2449 | GTTTAGCTGGGGAGTTTTCTGACTC | 3052 |
| 4 | AGUCAGAAAACUCCCCAGCUAAACA | 2450 | TGTTTAGCTGGGGAGTTTTCTGACT | 3054 |
| 5 | GUCAGAAAACUCCCCAGCUAAACAC | 2451 | GTGTTTAGCTGGGGAGTTTTCTGAC | 3055 |
| 6 | UCAGAAAACUCCCCAGCUAAACACC | 2452 | GGTGTTTAGCTGGGGAGTTTTCTGA | 3056 |
| 7 | CAGAAAACUCCCCAGCUAAACACCC | 2453 | GGGTGTTTAGCTGGGGAGTTTTCTG | 3057 |
| 8 | AGAAAACUCCCCAGCUAAACACCCG | 2454 | CGGGTGTTTAGCTGGGGAGTTTTCT | 3058 |
| 9 | GAAAACUCCCCAGCUAAACACCCGU | 2455 | ACGGGTGTTTAGCTGGGGAGTTTTC | 3059 |
| 10 | AAAACUCCCCAGCUAAACACCCGUA | 2456 | TACGGGTGTTTAGCTGGGGAGTTTT | 3060 |
| 11 | AAACUCCCCAGCUAAACACCCGUAA | 2457 | TTACGGGTGTTTAGCTGGGGAGTTT | 3061 |
| 12 | AACUCCCCAGCUAAACACCCGUAAG | 2458 | CTTACGGGTGTTTAGCTGGGGAGTT | 3062 |
| 13 | ACUCCCCAGCUAAACACCCGUAAGA | 2459 | TCTTACGGGTGTTTAGCTGGGGAGT | 3063 |
| 14 | CUCCCCAGCUAAACACCCGUAAGAC | 2460 | GTCTTACGGGTGTTTAGCTGGGGAG | 3064 |
| 15 | UCCCCAGCUAAACACCCGUAAGACU | 2461 | AGTCTTACGGGTGTTTAGCTGGGGA | 3065 |
| 16 | CCCCAGCUAAACACCCGUAAGACUU | 2462 | AAGTCTTACGGGTGTTTAGCTGGGG | 2066 |
| 17 | CCCAGCUAAACACCCGUAAGACUUC | 2463 | GAAGTCTTACGGGTGTTTAGCTGGG | 3067 |
| 18 | CCAGCUAAACACCCGUAAGACUUCA | 2464 | TGAAGTCTTACGGGTGTTTAGCTGG | 3068 |
| 19 | CAGCUAAACACCCGUAAGACUUCAU | 2465 | ATGAAGTCTTACGGGTGTTTAGCTG | 3069 |
| 20 | AGCUAAACACCCGUAAGACUUCAUA | 2466 | TATGAAGTCTTACGGGTGTTTAGCT | 3070 |
| 21 | GCUAAACACCCGUAAGACUUCAUAC | 2467 | GTATGAAGTCTTACGGGTGTTTAGC | 3071 |
| 22 | CUAAACACCCGUAAGACUUCAUACA | 2468 | TGTATGAAGTCTTACGGGTGTTTAG | 3072 |
| 23 | UAAACACCCGUAAGACUUCAUACAA | 2469 | TTGTATGAAGTCTTACGGGTGTTTA | 3073 |
| 24 | AAACACCCGUAAGACUUCAUACCAC | 2470 | GTTGTATGAAGTCTTACGGGTGTTT | 3064 |
| 25 | AACACCCGUAAGACUUCAUACAACA | 2471 | TGTTGTATGAAGTCTTACGGGTGTT | 3065 |
| 26 | ACACCCGUAAGACUUCAUACAACAC | 2472 | GTGTTGTATGAAGTCTTACGGGTGT | 3076 |
| 27 | CACCCGUAAGACUUCAUACAACACA | 2473 | TGTGTTGTATGAAGTCTTACGGGTG | 3077 |
| 28 | ACCCGUAAGACUUCAUACAACACAA | 2474 | TTGTGTTGTATGAAGTCTTACGGGT | 3078 |
| 29 | CCCGUAAGACUUCAUACAACACAAU | 2475 | ATTGTFTTGTATGAAGTCTTACGGG | 3079 |
| 63 | UGUGAUGAUCACAGCUGCCAAGGCU | 2476 | AGCCTTGGCAGCTGTGATCATCACA | 3080 |
| 64 | GUGAUGAUCACAGCUGCCAAGGCUA | 2477 | TAGCCTTGGCAGCTGTGATCATCAC | 3081 |
| 65 | UGAUGAUCACAGCUGCCAAGGCUAC | 2478 | GTAGCCTTGGCAGCTGTGATCATCA | 3082 |
| 66 | GAUGAUCACAGCUGCCAAGGCUACC | 2479 | GGTAGCCTTGGCAGCTGTGATCATC | 3083 |
| 67 | AUGAUCACAGCUGCCAAGGCUACCU | 2480 | AGGTAGCCTTGGCAGCTGTGATCAT | 3084 |
| 68 | UGAUCACAGCUGCCAAGGCUACCUA | 2481 | TAGGTAGCCTTGGCAGCTGTGATCA | 3085 |
| 69 | GAUCACAGCUGCCAAGGCUACCUAA | 2482 | TTAGGTAGCCTTGGCAGCTGTGATC | 3086 |
| 70 | AUCACAGCUGCCAAGGCUACCUAAA | 2483 | TTTAGGTAGCCTTGGCAGCTGTGAT | 3087 |
| 71 | UCACAGCUGCCAAGGCUACCUAAAA | 2484 | TTTTAGGTAGCCTTGGCAGCTGTGA | 3088 |
| 72 | CACAGCUGCCAAGGCUACCUAAAAG | 2485 | CTTTTAGGTAGCCTTGGCAGCTGTG | 3089 |
| 73 | ACAGCUGCCAAGGCUACCUAAAAGA | 2486 | TCTTTTAGGTAGCCTTGGCAGCTGT | 3090 |
| 74 | CAGCUGCCAAGGCUACCUAAAAGAA | 2487 | TTCTTTTAGGTAGCCTTGGCAGCTG | 3091 |
| 75 | AGCUGCCAAGGCUACCUAAAAGAAG | 2488 | CTTCTTTTAGGTAGCCTTGGCAGCT | 3092 |
| 76 | GCUGCCAAGGCUACCUAAAAGAAGA | 2489 | TCTTCTTTTAGGTAGCCTTGGCAGC | 3093 |
| 77 | CUGCCAAGGCUACCUAAAAGAAGAC | 2490 | GTCTTCTTTTAGGTAGCCTTGGCAG | 3094 |
| 78 | UGCCAAGGCUACCUAAAAGAAGACA | 2491 | TGTCTTCTTTTAGGTAGCCTTGGCA | 3095 |
| 79 | GCCAAGGCUACCUAAAAGAAGACAG | 2492 | CTGTCTTCTTTTAGGTAGCCTTGGC | 3096 |
| 80 | CCAAGGCUACCUAAAAGAAGACAGU | 2493 | ACTGTCTTCTTTTAGGTAGCCTTGG | 3097 |
| 81 | CAAGGCUACCUAAAAGAAGACAGUU | 2494 | AACTGTCTTCTTTTAGGTAGCCTTG | 3098 |
| 98 | AGACAGUUAUCUCAUAUUUGGCUGC | 2495 | GCAGCCAAATATGAGATAACTGTCT | 3099 |
| 99 | GACAGUUAUCUCAUAUUUGGCUGCC | 2496 | GGCAGCCAAATATGAGATAACTGTC | 3100 |
| 100 | ACAGUUAUCUCAUAUUUGGCUGCCA | 2497 | TGGCAGCCAAATATGAGATAACTGT | 3101 |
| 101 | CAGUUAUCUCAUAUUUGGCUGCCAG | 2498 | CTGGCAGCCAAATATGAGATAACTG | 3102 |
| 102 | AGUUAUCUCAUAUUUGGCUGCCAGC | 2499 | GCTGGCAGCCAAATATGAGATAACT | 3103 |
| 103 | GUUAUCUCAUAUUUGGCUGCCAGCU | 2500 | AGCTGGCAGCCAAATATGAGATAAC | 3104 |
| 104 | UUAUCUCAUAUUUGGCUGCCAGCUU | 2501 | AAGCTGGCAGCCAAATATGAGATAA | 3105 |
| 105 | UAUCUCAUAUUUGGCUGCCAGCUUU | 2502 | AAAGCTGGCAGCCAAATATGAGATA | 3106 |
| 106 | AUCUCAUAUUUGGCUGCCAGCUUUU | 2503 | AAAAGCTGGCAGCCAAATATGAGAT | 3107 |
| 107 | UCUCAUAUUUGGCUGCCAGCUUUUU | 2504 | AAAAAGCTGGCAGCCAAATATGAGA | 3108 |
| 108 | CUCAUAUUUGGCUGCCAGCUUUUUA | 2505 | TAAAAAGCTGGCAGCCAAATATGAG | 3109 |
| 109 | UCAUAUUUGGCUGCCAGCUUUUUAU | 2506 | ATAAAAAGCTGGCAGCCAAATATGA | 3110 |
| 110 | CAUAUUUGGCUGCCAGCUUUUUAUC | 2507 | GATAAAAAGCTGGCAGCCAAATATG | 3111 |
| 111 | AUAUUUGGCUGCCAGCUUUUUAUCU | 2508 | AGATAAAAAGCTGGCAGCCAAATAT | 3112 |
| 112 | UAUUUGGCUGCCAGCUUUUUAUCUU | 2509 | AAGATAAAAAGCTGGCAGCCAAATA | 3113 |
| 113 | AUUUGGCUGCCAGCUUUUUAUCUUU | 2510 | AAAGATAAAAAGCTGGCAGCCAAAT | 3114 |
| 114 | UUUGGCUGCCAGCUUUUUAUCUUUC | 2511 | GAAAGATAAAAAGCTGGCAGCCAAA | 3115 |
| 115 | UUGGCUGCCAGCUUUUUAUCUUUCU | 2512 | AGAAAGATAAAAAGCTGGCAGCCAA | 3116 |
| 116 | UGGCUGCCAGCUUUUUAUCUUUCUC | 2513 | GAGAAAGATAAAAAGCTGGCAGCCA | 3117 |
| 117 | GGCUGCCAGCUUUUUAUCUUUCUCU | 2514 | AGAGAAAGATAAAAAGCTGGCAGCC | 3118 |
| 118 | GCUGCCAGCUUUUUAUCUUUCUCUC | 2515 | GAGAGAAAGATAAAAAGCTGGCAGC | 3119 |
| 119 | CUGCCAGCUUUUUAUCUUUCUCUCG | 2516 | CGAGAGAAAGATAAAAAGCTGGCAG | 3120 |
| 120 | UGCCAGCUUUUUAUCUUUCUCUCGA | 2517 | TCGAGAGAAAGATAAAAAGCTGGCA | 3121 |
| 121 | GCCAGCUUUUUAUCUUUCUCUCGAC | 2518 | GTCGAGAGAAAGATAAAAAGCTGGC | 3122 |
| 122 | CCAGCUUUUUAUCUUUCUCUCGACC | 2519 | GGTCGAGAGAAAGATAAAAAGCTGG | 3123 |
| 123 | CAGCUUUUUAUCUUUCUCUCGACCA | 2520 | TGGTCGAGAGAAAGATAAAAAGCTG | 3124 |
| 124 | AGCUUUUUAUCUUUCUCUCGACCAC | 2521 | GTGGTCGAGAGAAAGATAAAAAGCT | 3125 |
| 125 | GCUUUUUAUCUUUCUCUCGACCACU | 2522 | AGTGGTCGAGAGAAAGATAAAAAGC | 3126 |
| 126 | CUUUUUAUCUUUCUCUCGACCACUU | 2523 | AAGTGGTCGAGAGAAAGATAAAAAG | 3127 |
| 132 | AUCUUUCUCUCGACCACUUAAAACU | 2524 | AGTTTTAAGTGGTCGAGAGAAAGAT | 3128 |
| 133 | UCUUUCUCUCGACCACUUAAAACUU | 2525 | AAGTTTTAAGTGGTCGAGAGAAAGA | 3129 |
| 134 | CUUUCUCUCGACCACUUAAAACUUU | 2526 | GAAGTTTTAAGTGGTCGAGAGAAAG | 3130 |
| 135 | UUUCUCUCGACCACUUAAAACUUCA | 2527 | TGAAGTTTTAAGTGGTCGAGAGAAA | 3131 |
| 136 | UUCUCUCGACCACUUAAAACUUCAG | 2528 | CTGAGGTTTTAAGTGGTCGAGAGAA | 3132 |
| 137 | UCUCUCGACCACUUAAAACUUCAGA | 2529 | TCTGAAGTTTTAAGTGGTCGAGAGA | 3133 |
| 138 | CUCUCGACCACUUAAAACUUCAGAC | 2530 | GTCTGAAGTTTTAAGTGGTCGAGAG | 3134 |
| 139 | UCUCGACCACUUAAAACUUCAGACU | 2531 | AGTCTGAAGTTTTAAGTGGTCGAGA | 3135 |
| 140 | CUCGACCACUUAAAACUUCAGACUU | 2532 | AAGTCTGAAGTTTTAAGTGGTCGAG | 3136 |
| 141 | UCGACCACUUAAAACUUCAGACUUC | 2533 | GAAGTCTGAAGTTTTAAGTGGTCGA | 3137 |
| 142 | CGACCACUUAAAACUUCAGACUUCC | 2534 | GGAAGTCTGAAGTTTTAAGTGGTCG | 3138 |
| 143 | GACCACUUAAAACUUCAGACUUCCU | 2535 | AGGAAGTCTGAAGTTTTAAGTGGTC | 3139 |
| 144 | ACCACUUAAAACUUCAGACUUCCUG | 2536 | CAGGAAGTCTGAAGTTTTAAGTGGT | 3140 |
| 145 | CCACUUAAAACUUCAGACUUCCUGU | 2537 | ACAGGAAGTCTGAAGTTTTAAGTGG | 3141 |
| 147 | ACUUAAAACUUCAGACUUCCUGUCC | 2538 | GGACAGGAAGTCTGAAGTTTTAAGT | 3142 |
| 148 | CUUAAAACUUCAGACUUCCUGUCCU | 2539 | AGGACAGGAAGTCTGAAGTTTTAAG | 3143 |
| 149 | UUAAAACUUCAGACUUCCUGUCCUG | 2540 | CAGGACAGGAAGTCTGAAGTTTTAA | 3144 |
| 150 | UAAAACUUCAGACUUCCUGUCCUGC | 2541 | GCAGGACAGGAAGTCTGAAGTTTTA | 3145 |
| 151 | AAAACUUCAGACUUCCUGUCCUGCU | 2542 | AGCAGGACAGGAAGTCTGAAGTTTT | 3146 |
| 152 | AAACUUCAGACUUCCUGUCCUGCUG | 2543 | CAGCAGGACAGGAAGTCTGAAGTTT | 3147 |
| 153 | AACUUCAGACUUCCUGUCCUGCUGG | 2544 | CCAGCAGGACAGGAAGTCTGAAGTT | 3148 |
| 154 | ACUUCAGACUUCCUGUCCUGCUGGU | 2545 | ACCAGCAGGACAGGAAGTCTGAAGT | 3149 |
| 155 | CUUCAGACUUCCUGUCCUGCUGGUA | 2546 | TACCAGCAGGACAGGAAGTCTGAAG | 3150 |
| 156 | UUCAGACUUCCUGUCCUGCUGGUAU | 2547 | ATACCAGCAGGACAGGAAGTCTGAA | 3151 |
| 157 | UCAGACUUCCUGUCCUGCUGGUAUC | 2548 | GATACCAGCAGGACAGGAAGTCTGA | 3152 |
| 158 | CAGACUUCCUGUCCUGCUGGUAUCA | 2549 | TGATACCAGCAGGACAGGAAGTCTG | 3153 |
| 159 | AGACUUCCUGUCCUGCUGGUAUCAU | 2550 | ATGATACCAGCAGGACAGGAAGTCT | 3154 |
| 160 | GACUUCCUGUCCUGCUGGUAUCAUG | 2551 | CATGATACCAGCAGGACAGGAAGTC | 3155 |
| 161 | ACUUCCUGUCCUGCUGGUAUCAUGG | 2552 | CCATGATACCAGCAGGACAGGAAAG | 3156 |
| 162 | CUUCCUGUCCUGCUGGUAUCAUGGA | 2553 | TCCATGATACCAGCAGGACAGGAAG | 3157 |
| 163 | UUCCUGUCCUGCUGGUAUCAUGGAG | 2554 | CTCCATGATACCAGCAGGACAGGAA | 3158 |
| 164 | UCCUGUCCUGCUGGUAUCAUGGAGA | 2555 | TCTCCATGATACCAGCAGGACAGGA | 3159 |
| 165 | CCUGUCCUGCUGGUAUCAUGGAGAA | 2556 | TTCTCCATGATACCAGCAGGACAGG | 3160 |
| 166 | CUGUCCUGCUGGUAUCAUGGAGAAA | 2557 | TTTCTCCATGATACCAGCAGGACAG | 3161 |
| 167 | UGUCCUGCUGGUAUCAUGGAGAAAG | 2558 | CTTTCTCCATGATACCAGCAGGACA | 3162 |
| 168 | GUCCUGCUGGUAUCAUGGAGAAAGU | 2559 | ACTTTCTCCATGATACCAGCAGGAC | 3163 |
| 169 | UCCUGCUGGUAUCAUGGAGAAAGUC | 2560 | GACTTTCTCCATGATACCAGCAGGA | 3164 |
| 170 | CCUGCUGGUAUCAUGGAGAAAGUCC | 2561 | GGACTTTCTCCATGATACCAGCAGG | 3165 |
| 180 | UCAUGGAGAAAGUCCAAUACCUCAC | 2562 | GTGAGGTATTGGACTTTCTCCATGA | 3166 |
| 181 | CAUGGAGAAAGUCCAAUACCUCACU | 2563 | AGTGAGGTATTGGACTTTCTCCATG | 3167 |
| 182 | AUGGAGAAAGUCCAAUACCUCACUC | 2564 | GAGTGAGGTATTGGACTTTCTCCAT | 3168 |
| 183 | UGGAGAAAGUCCAAUACCUCACUCG | 2565 | CGAGTGAGGTATTGGACTTTCTCCA | 3169 |
| 184 | GGAGAAAGUCCAAUACCUCACUCGC | 2566 | GCGAGTGAGGTATTGGACTTTCTCC | 3170 |
| 185 | GAGAAAGUCCAAUACCUCACUCGCU | 2567 | AGCGAGTGAGGTATTGGACTTTCTC | 3171 |
| 186 | AGAAAGUCCAAUACCUCACUCGCUC | 2568 | GAGCGAGTGAGGTATTGGACTTTCT | 3172 |
| 187 | GAAAGUCCAAUACCUCACUCGCUCA | 2569 | TGAGCGAGTGAGGTATTGGACTTTC | 3173 |
| 188 | AAAGUCCAAUACCUCACUCGCUCAG | 2570 | CTGAGCGAGTGAGGTATTGGACTTT | 3174 |
| 189 | AAGUCCAAUACCUCACUCGCUCAGC | 2571 | GCTGAGCGAGTGAGGTATTGGACTT | 3175 |
| 190 | AGUCCAAUACCUCACUCGCUCAGCU | 2572 | AGCTGAGCGAGTGAGGTATTGGACT | 3176 |
| 191 | GUCCAAUACCUCACUCGCUCAGCUA | 2573 | TAGCTGAGCGAGTGAGGTATTGGAC | 3177 |
| 192 | UCCAAUACCUCACUCGCUCAGCUAU | 2574 | ATAGCTGAGCGAGTGAGGTATTGGA | 3178 |
| 193 | CCAAUACCUCACUCGCUCAGUCAUA | 2575 | TATAGCTGAGCGAGTGAGGTATTGG | 3178 |
| 194 | CAAUACCUCACUCGCUCAGCUAUAA | 2576 | TTATAGCTGAGCGAGTGAGGTATTG | 3180 |
| 195 | AAUACCUCACUCGCUCAGCUAUAAG | 2577 | CTTATAGCTGAGCGAGTGAGGTATT | 3181 |
| 196 | AUACCUCACUCGCUCAGCUAUAAGA | 2578 | TCTTATAGCTGAGCGAGTGAGGTAT | 3182 |
| 197 | UACCUCACUCGCUCAGCUAUAAGAA | 2579 | TTCTTATAGCTGAGCGAGTGAGGTA | 3183 |
| 198 | ACCUCACUCGCUCAGCUAUAAGAAG | 2580 | CTTCTTATAGCTGAGCGAGTGAGGT | 3184 |
| 199 | CCUCACUCGCUCAGCUAUAAGAAGA | 2581 | TCTTCTTATAGCTGAGCGAGTGAGG | 3185 |
| 200 | CUCACUCGCUCAGCUAUAAGAAGAG | 2582 | CTCTTCTTATAGCTGAGCGAGTGAG | 3186 |
| 201 | UCACUCGCUCAGCUAUAAGAAGAGC | 2583 | GCTCTTCTTATAGCTGAGCGAGTGA | 3187 |
| 202 | CACUCGCUCAGCUAUAAGAAGAGCC | 2584 | GGCTCTTCTTATAGCTGAGCGAGTG | 3188 |
| 203 | ACUCGCUCAGCUAUAAGAAGAGCCU | 2585 | AGGCTCTTCTTATAGCTGAGCGAGT | 3189 |
| 204 | CUCGCUCAGCUAUAAGAAGAGCCUC | 2586 | GAGGCTCTTCTTATAGCTGAGCGAG | 3190 |
| 205 | UCGCUCAGCUAUAAGAAGAGCCUCA | 2587 | TGAGGCTCTTCTTATAGCTGAGCGA | 3191 |
| 206 | CGCUCAGCUAUAAGAAGAGCCUCAA | 2588 | TTGAGGCTCTTCTTATAGCTGAGCG | 3192 |
| 207 | GCUCAGCUAUAAGAAGAGCCUCAAC | 2589 | GTTGAGGCTCTTCTTATAGCTGAGC | 3193 |
| 208 | CUCAGCUAUAAGAAGAGCCUCAACC | 2590 | GGTTGAGGCTCTTCTTATAGCTGAG | 3194 |
| 209 | UCAGCUAUAAGAAGAGCCUCAACCA | 2591 | TGGTTGAGGCTCTTCTTATAGCTGA | 3195 |
| 210 | CAGCUAUAAGAAGAGCCUCAACCAU | 2592 | ATGGTTGAGGCTCTTCTTATAGCTG | 3196 |
| 211 | AGCUAUAAGAAGAGCCUCAACCAUU | 2593 | AATGGTTGAGGCTCTTCTTATAGCT | 3197 |
| 212 | GCUAUAAGAAGAGCCUCAACCAUUG | 2594 | CAATGGTTGAGGCTCTTCTTATAGC | 3198 |
| 213 | CUAUAAGAAGAGCCUCAACCAUUGG | 2595 | TCAATGGTTGAGGCTCTTCTTATAG | 3199 |
| 214 | UAUAAGAAGAGCCUCAACCAUUGAA | 2596 | TTCAATGGTTGAGGCTCTTCTTATA | 3200 |
| 215 | AUAAGAAGAGCCUCAACCAUUGAAA | 2597 | TTTCAATGGTTGAGGCTCTTCTTAT | 3201 |
| 216 | UAAGAAGAGCCUCAACCAUUGAAAU | 2598 | ATTTCAATGGTTGAGGCTCTTCTTA | 3202 |
| 217 | AAGAAGAGCCUCAACCAUUGAAAUG | 2599 | CATTTCAATGGTTGAGGCTCTTCTT | 3203 |
| 218 | AGAAGAGCCUCAACCAUUGAAAUGC | 2600 | GCATTTCAATGGTTGAGGCTCTTCT | 3204 |
| 219 | GAAGAGCCUCAACCAUUGAAAUGCC | 2601 | GGCATTTCAATGGTTGAGGCTCTTC | 3205 |
| 220 | AAGAGCCUCAACCAUUGAAAUGCCU | 2602 | AGGCATTTCAATGGTTGAGGCTCTT | 3206 |
| 221 | AGAGCCUCAACCAUUGAAAUGCCUC | 2603 | GAGGCATTTCAATGGTTGAGGCTCT | 3207 |
| 222 | GAGCCUCAACCAUUGAAAUGCCUCA | 2604 | TGAGGCATTTCAATGGTTGAGGCTC | 3208 |
| 223 | AGCCUCAACCAUUGAAAUGCCUCAA | 2605 | TTGAGGCATTTCAATGGTTGAGGCT | 3209 |
| 224 | GCCUCAACCAUUGAAAUGCCUCACC | 2606 | GTTGAGGCATTTCAATGGTTGAGGC | 3210 |
| 225 | CCUCAACCAUUGAAAUGCCUCAACA | 2607 | TGTTGAGGCATTTCAATGGTTGAGG | 3211 |
| 226 | CUCAACCAUUGAAAUGCCUCAACAA | 2608 | TTGTTGAGGCATTTCAATGGTTGAG | 3212 |
| 227 | UCAACCAUUGAAAUGCCUCAACAAG | 2609 | CTTGTTGAGGCATTTCAATGGTTGA | 3213 |
| 228 | CAACCAUUGAAAUGCCUCAACAAGC | 2610 | GCTTGTTGAGGCATTTCAATGGTTG | 3214 |
| 229 | AACCAUUGAAAUGCCUCAACAAGCA | 2611 | TGCTTGTTGAGGCATTTCAATGGTT | 3215 |
| 230 | ACCAUUGAAAUGCCUCAACAAGCAC | 2612 | GTGCTTGTTGAGGCATTTCAATGGT | 3216 |
| 231 | CCAUUGAAAUGCCUCAACAAGCACG | 2613 | CGTGCTTGTTGAGGCATTTCAATGG | 3217 |
| 232 | CAUUGAAAUGCCUCAACAAGCACGU | 2614 | ACGTGCTTGTTGAGGCATTTCAATG | 3218 |
| 233 | AUUGAAAUGCCUCAACAAGCACGUC | 2615 | GACGTGCTTGTTGAGGCATTTCAAT | 3219 |
| 234 | UUGAAAUGCCUCAACAAGCACGUCA | 2616 | TGACGTGCTTGTTGAGGCATTTCAA | 3220 |
| 235 | UGAAAUGCCUCAACAAGCACGUCAA | 2617 | TTGACGTGCTTGTTGAGGCATTTCA | 3221 |
| 236 | GAAAUGCCUCAACAAGCACGUCAAA | 2618 | TTTGACGTGCTTGTTGAGGCATTTC | 3222 |
| 237 | AAAUGCCUCAACAAGCACGUCAAAA | 2619 | TTTTGACGTGCTTGTTGAGGCATTT | 3223 |
| 238 | AAUGCCUCAACAAGCACGUCAAAAG | 2620 | CTTTTGACGTGCTTGTTGAGGCATT | 3224 |
| 239 | AUGCCUCAACAAGCACGUCAAAAGC | 2621 | GCTTTTGACGTGCTTGTTGAGGCAT | 3225 |
| 240 | UGCCUCAACAAGCACGUCAAAAGCU | 2622 | AGCTTTTGACGTGCTTGTTGAGGCA | 3226 |
| 241 | GCCUCAACAAGCACGUCAAAAGCUA | 2623 | TAGCTTTTGACGTGCTTGTTGAGGC | 3227 |
| 242 | CCUCAACAAGCACGUCAAAAGCUAC | 2624 | GTAGCTTTTGACGTGCTTGTTGAGG | 3228 |
| 243 | CUCAACAAGCACGUCAAAAGCUACA | 2625 | TGTAGCTTTTGACGTGCTTGTTGAG | 3229 |
| 244 | UCAACAAGCACGUCAAAAGCUACAG | 2626 | CTGTAGCTTTTGACGTGCTTGTTGA | 3230 |
| 245 | CAACAAGCACGUCAAAAGCUACAGA | 2627 | TCTGTAGCTTTTGACGTGCTTGTTG | 3231 |
| 246 | AACAAGCACGUCAAAAGCUACAGAA | 2628 | TTCTGTAGCTTTTGACGTGCTTGTT | 3232 |
| 247 | ACAAGCACGUCAAAAGCUACAGAAU | 2629 | ATTCTGTAGCTTTTGACGTGCTTGT | 3233 |
| 248 | CAAGCACGUCAAAAGCUACAGAAUC | 2630 | GATTCTGTAGCTTTTGACGTGCTTG | 3234 |
| 249 | AAGCACGUCAAAAGCUACAGAAUCU | 2631 | AGATTCTGTAGCTTTTGACGTGCTT | 3235 |
| 250 | AGCACGUCAAAAGCUACAGAAUCUA | 2632 | TAGATTCTGTAGCTTTTGACGTGCT | 3236 |
| 251 | GCACGUCAAAAGCUACAGAAUCUAU | 2633 | ATAGATTCTGTAGCTTTTGACGTGC | 3237 |
| 252 | CACGUCAAAAGCUACAGAAUCUAUU | 2634 | AATAGATTCTGTAGCTTTTGACGTG | 3238 |
| 253 | ACGUCAAAAGCUACAGAAUCUAUUU | 2635 | AAATAGATTCTGTAGCTTTTGACGT | 3239 |
| 254 | CGUCAAAAGCUACAGAAUCUAUUUA | 2636 | TAAATAGATTCTGTAGCTTTTGACG | 3240 |
| 255 | GUCAAAAGCUACAGAAUCUAUUUAU | 2637 | ATAAATAGATTCTGTAGCTTTTGAC | 3241 |
| 256 | UCAAAAGCUACAGAAUCUAUUUAUC | 2638 | GATAAATAGATTCTGTAGCTTTTGA | 3242 |
| 257 | CAAAAGCUACAGAAUCUAUUUAUCA | 2639 | TGATAAATAGATTCTGTAGCTTTTG | 3243 |
| 258 | AAAAGCUACAGAAUCUAUUUAUCAA | 2640 | TTGATAAATAGATTCTGTAGCTTTT | 3244 |
| 259 | AAAGCUACAGAAUCUAUUUAUCAAU | 2641 | ATTGATAAATAGATTCTGTAGCTTT | 3245 |
| 260 | AAGCUACAGAAUCUAUUUAUCAAUU | 2642 | AATTGATAAATAGATTCTGTAGCTT | 3246 |
| 261 | AGCUACAGAAUCUAUUUAUCAAUUU | 2643 | AAATTGATAAATAGATTCTGTAGCT | 3247 |
| 262 | GCUACAGAAUCUAUUUAUCAAUUUC | 2644 | GAAATTGATAAATAGATTCTGTAGC | 3248 |
| 263 | CUACAGAAUCUAUUUAUCAAUUUCU | 2645 | AGAAATTGATAAATAGATTCTGTAG | 3249 |
| 264 | UACAGAAUCUAUUUAUCAAUUUCUG | 2646 | CAGAAATTGATAAATAGATTCTGTA | 3250 |
| 265 | ACAGAAUCUAUUUAUCAAUUUCUGU | 2647 | ACAGAAATTGATAAATAGATTCTGT | 3251 |
| 266 | CAGAAUCUAUUUAUCAAUUUCUGUC | 2648 | GACAGAAATTGATAAATAGATTCTG | 3252 |
| 267 | AGAAUCUAUUUAUCAAUUUCUGUCU | 2649 | AGACAGAAATTGATAAATAGATTCT | 3253 |
| 268 | GAAUCUAUUUAUCAAUUUCUGUCUC | 2650 | GAGACAGAAATTGATAAATAGATTC | 3254 |
| 269 | AAUCUAUUUAUCAAUUUCUGUCUCA | 2651 | TGAGACAGAAATTGATAAATAGATT | 3255 |
| 270 | AUCUAUUUAUCAAUUUCUGUCUCAU | 2652 | ATGAGACAGAAATTGATAAATAGAT | 3256 |
| 271 | UCUAUUUAUCAAUUUCUGUCUCAUC | 2653 | GATGAGACAGAAATTGATAAATAGA | 3257 |
| 272 | CUAUUUAUCAAUUUCUGUCUCAUCU | 2654 | AGATGAGACAGAAATTGATAAATAG | 3258 |
| 273 | UAUUUAUCAAUUUCUGUCUCAUCUU | 2655 | AAGATGAGACAGAAATTGATAAATA | 3259 |
| 274 | AUUUAUCAAUUUCUGUCUCAUCUUA | 2656 | TAAGATGAGACAGAAATTGATAAAT | 3260 |
| 275 | UUUAUCAAUUUCUGUCUCAUCUUAA | 2657 | TTAAGATGAGACAGAAATTGATAAA | 3261 |
| 276 | UUAUCAAUUUCUGUCUCAUCUUAAU | 2658 | ATTAAGATGAGACAGAAATTGATAA | 3262 |
| 277 | UAUCAAUUUCUGUCUCAUCUUAAUA | 2659 | TATTAAGATGAGACAGAAATTGATA | 3263 |
| 278 | AUCAAUUUCUGUCUCAUCUUAAUAU | 2660 | ATATTAAGATGAGACAGAAATTGAT | 3264 |
| 279 | UCAAUUUCUGUCUCAUCUUAAUAUG | 2661 | CATATTAAGATGAGACAGAAATTGA | 3265 |
| 280 | CAAUUUCUGUCUCAUCUUAAUAUGU | 2662 | ACATATTAAGATGAGACAGAAATTG | 3266 |
| 281 | AAUUUCUGUCUCAUCUUAAUAUGUC | 2663 | GACATATTAAGATGAGACAGAAATT | 3267 |
| 282 | AUUUCUGUCUCAUCUUAAUAUGUCU | 2664 | AGACATATTAAGATGAGACAGAAAT | 3268 |
| 283 | UUUCUGUCUCAUCUUAAUAUGUCUC | 2665 | GAGACATATTAAGATGAGACAGAAA | 3269 |
| 284 | UUCUGUCUCAUCUUAAUAUGUCUCU | 2666 | AGAGACATATTAAGATGAGACAGAA | 3270 |
| 285 | UCUGUCUCAUCUUAAUAUGUCUCUU | 2667 | AAGAGACATATTAAGATGAGACAGA | 3271 |
| 286 | CUGUCUCAUCUUAAUAUGUCUCUUG | 2668 | CAAGAGACATATTAAGATGAGACAG | 3272 |
| 287 | UGUCUCAUCUUAAUAUGUCUCUUGC | 2669 | GCAAGAGACATATTAAGATGAGACA | 3273 |
| 288 | GUCUCAUCUUAAUAUGUCUCUUGCU | 2670 | AGCAAGAGACATATTAAGATGAGAC | 3274 |
| 289 | UCUCAUCUUAAUAUGUCUCUUGCUG | 2671 | CAGCAAGAGACATATTAAGATGAGA | 3275 |
| 290 | CUCAUCUUAAUAUGUCUCUUGCUGA | 2672 | TCAGCAAGAGACATATTAAGATGAG | 3276 |
| 291 | UCAUCUUAAUAUGUCUCUUGCUGAU | 2673 | ATCAGCAAGAGACATATTAAGATGA | 3277 |
| 292 | CAUCUUAAUAUGUCUCUUGCUGAUC | 2674 | GATCAGCAAGAGACATATTAAGATG | 3278 |
| 293 | AUCUUAAUAUGUCUCUUGCUGAUCU | 2675 | AGATCAGCAAGAGACATATTAAGAT | 3279 |
| 294 | UCUUAAUAUGUCUCUUGCUGAUCUG | 2676 | CAGATCAGCAAGAGACATATTAAGA | 3280 |
| 295 | CUUAAUAUGUCUCUUGCUGAUCUGU | 2677 | ACAGATCAGCAAGAGACATATTAAG | 3281 |
| 343 | UUCUGCUACAACCUCUAGAUCUGCA | 2725 | TGCAGATCTAGAGGTTGTAGCAGAA | 3329 |
| 344 | UCUGCUACAACCUCUAGAUCUGCAG | 2726 | CTGCAGATCTAGAGGTTGTAGCAGA | 3330 |
| 345 | CUGCUACAACCUCUAGAUCUGCAGC | 2727 | GCTGCAGATCTAGAGGTTGTAGCAG | 3331 |
| 346 | UGCUACAACCUCUAGAUCUGCAGCU | 2728 | AGCTGCAGATCTAGAGGTTGTAGCA | 3332 |
| 347 | GCUACAACCUCUAGAUCUGCAGCUU | 2729 | AAGCTGCAGATCTAGAGGTTGTAGC | 3333 |
| 348 | CUACAACCUCUAGAUCUGCAGCUUG | 2730 | CAAGCTGCAGATCTAGAGGTTGTAG | 3334 |
| 349 | UACAACCUCUAGAUCUGCAGCUUGC | 2731 | GCAAGCTGCAGATCTAGAGGTTGTA | 3335 |
| 350 | ACAACCUCUAGAUCUGCAGCUUGCC | 2732 | GGCAAGCTGCAGATCTAGAGGTTGT | 3336 |
| 351 | CAACCUCUAGAUCUGCAGCUUGCCA | 2733 | TGGCAAGCTGCAGATCTAGAGGTTG | 3337 |
| 352 | AACCUCUAGAUCUGCAGCUUGCCAC | 2734 | GTGGCAAGCTGCAGATCTAGAGGTT | 3338 |
| 353 | ACCUCUAGAUCUGCAGCUUGCCACA | 2735 | TGTGGCAAGCTGCAGATCTAGAGGT | 3339 |
| 354 | CCUCUAGAUCUGCAGCUUGCCACAU | 2736 | ATGTGGCAAGCTGCAGATCTAGAGG | 3340 |
| 355 | CUCUAGAUCUGCAGCUUGCCACAUC | 2737 | GATGTGGCAAGCTGCAGATCTAGAG | 3341 |
| 356 | UCUAGAUCUGCAGCUUGCCACAUCA | 2738 | TGATGTGGCAAGCTGCAGATCTAGA | 3342 |
| 357 | CUAGAUCUGCAGCUUGCCACAUCAG | 2739 | CTGATGTGGCAAGCTGCAGATCTAG | 3343 |
| 358 | UAGAUCUGCAGCUUGCCACAUCAGC | 2740 | GCTGATGTGGCAAGCTGCAGATCTA | 3344 |
| 368 | GCUUGCCACAUCAGCUUAAAAUCUG | 2741 | CAGATTTTAAGCTGATGTGGCAAGC | 3345 |
| 369 | CUUGCCACAUCAGCUUAAAAUCUGU | 2742 | ACAGATTTTAAGCTGATGTGGCAAG | 3346 |
| 370 | UUGCCACAUCAGCUUAAAAUCUGUC | 2743 | GACAGATTTTAAGCTGATGTGGCAA | 3347 |
| 371 | UGCCACAUCAGCUUAAAAUCUGUCA | 2744 | TGACAGATTTTAAGCTGATGTGGCA | 3348 |
| 372 | GCCACAUCAGCUUAAAAUCUGUCAU | 2745 | ATGACAGATTTTAAGCTGATGTGGC | 3349 |
| 373 | CCACAUCAGCUUAAAAUCUGUCAUC | 2746 | GATGACAGATTTTAAGCTGATGTGG | 3350 |
| 374 | CACAUCAGCUUAAAAUCUGUCAUCC | 2747 | GGATGACAGATTTTAAGCTGATGTG | 3351 |
| 375 | ACAUCAGCUUAAAAUCUGUCAUCCC | 2748 | GGGATGACAGATTTTAAGCTGATGT | 3352 |
| 376 | CAUCAGCUUAAAAUCUGUCAUCCCA | 2749 | TGGGATGACAGATTTTAAGCTGATG | 3353 |
| 377 | AUCAGCUUAAAAUCUGUCAUCCCAU | 2750 | ATGGGATGACAGATTTTAAGCTGAT | 3354 |
| 378 | UCAGCUUAAAAUCUGUCAUCCCAUG | 2751 | CATGGGATGACAGATTTTAAGCTGA | 3355 |
| 379 | CAGCUUAAAAUCUGUCAUCCCAUGC | 2752 | GCATGGGATGACAGATTTTAAGCTG | 3356 |
| 380 | AGCUUAAAAUCUGUCAUCCCAUGCA | 2753 | TGCATGGGATGACAGATTTTAAGCT | 3357 |
| 381 | GCUUAAAAUCUGUCAUCCCAUGCAG | 2754 | CTGCATGGGATGACAGATTTTAAGC | 3358 |
| 382 | CUUAAAAUCUGUCAUCCCAUGCAGA | 2755 | TCTGCATGGGATGACAGATTTTAAG | 3359 |
| 383 | UUAAAAUCUGUCAUCCCAUGCAGAC | 2756 | GTCTGCATGGGATGACAGATTTTAA | 3360 |
| 384 | UAAAAUCUGUCAUCCCAUGCAGACA | 2757 | TGTCTGCATGGGATGACAGATTTTA | 3361 |
| 391 | UGUCAUCCCAUGCAGACAGGAAAAC | 2758 | GTTTTCCTGTCTGCATGGGATGACA | 3362 |
| 392 | GUCAUCCCAUGCAGACAGGAAAACA | 2759 | TGTTTTCCTGTCTGCATGGGATGAC | 3363 |
| 393 | UCAUCCCAUGCAGACAGGAAAACAA | 2760 | TTGTTTTCCTGTCTGCATGGGATGA | 3364 |
| 394 | CAUCCCAUGCAGACAGGAAAACAAU | 2761 | ATTGTTTTCCTGTCTGCATGGGATG | 3365 |
| 395 | AUCCCAUGCAGACAGGAAAACAAUA | 2762 | TATTGTTTTCCTGTCTGCATGGGAT | 3366 |
| 396 | UCCCAUGCAGACAGGAAAACAAUAU | 2763 | ATATTGTTTTCCTGTCTGCATGGGA | 3367 |
| 397 | CCCAUGCAGACAGGAAAACAAUAUU | 2764 | AATATTGTTTTCCTGTCTGCATGGG | 3368 |
| 398 | CCAUGCAGACAGGAAAACAAUAUUG | 2765 | CAATATTGTTTTCCTGTCTGCATGG | 3369 |
| 399 | CAUGCAGACAGGAAAACAAUAUUGU | 2766 | ACAATATTGTTTTCCTGTCTGCATG | 3370 |
| 400 | AUGCAGACAGGAAAACAAUAUUGUA | 2767 | TACAATATTGTTTTCCTGTCTGCAT | 3371 |
| 401 | UGCAGACAGGAAAACAAUAUUGUAU | 2768 | ATACAATATTGTTTTCCTGTCTGCA | 3373 |
| 426 | AACAGACCACUUCCUGAGUAGAAGA | 2769 | TCTTCTACTCAGGAAGTGGTCTGTT | 3374 |
| 427 | ACAGACCACUUCCUGAGUAGAAGAG | 2770 | CTCTTCTACTCAGGAAGTGGTCTGT | 3374 |
| 428 | CAGACCACUUCCUGAGUAGAAGAGU | 2771 | ACTCTTCTACTCAGGAAGTGGTCTG | 3375 |
| 430 | GACCACUUCCUGAGUAGAAGAGUUU | 2772 | AAACTCTTCTACTCAGGAAGTGGTC | 3376 |
| 431 | ACCACUUCCCUGAGUAGAAGAGUUC | 2773 | GAAACTCTTCTACTCAGGAAGTGGT | 3377 |
| 432 | CCACUUCCUGAGUAGAAGAGUUUCU | 2774 | AGAAACTCTTCTACTCAGGAAGTGG | 3378 |
| 445 | AGAAGAGUUUCUUUGUGAAAAGGUC | 2775 | GACCTTTTCACAAAGAAACTCTTCT | 3379 |
| 446 | GAAGAGUUUCUUUGUGAAAAGGUCA | 2776 | TGACCTTTTCACAAAGAAACTCTTC | 3380 |
| 447 | AAGAGUUUCUUUGUGAAAAGGUCAA | 2777 | TTGACCTTTTCACAAAGAAACTCTT | 3381 |
| 448 | AGAGUUUCUUUGUGAAAAGGUCAAG | 2778 | CTTGACCTTTTCACAAAGAAACTCT | 3382 |
| 449 | GAGUUUCUUUGUGAAAAGGUCAAGA | 2779 | TCTTGACCTTTTCACAAAGAAACTC | 3383 |
| 450 | AGUUUCUUUGUGAAAAGGUCAAGAU | 2780 | ATCTTGACCTTTTCACAAAGAAACT | 3384 |
| 451 | GUUCUUUGUGAAAAGGUCAAGAAUU | 2781 | AATCTTGACCTTTTCACAAAGAAAC | 3385 |
| 452 | UUUCUUUGUGAAAAGGUCAAGAUUA | 2782 | TAATCTTGACCTTTTCACAAAGAAA | 3386 |
| 453 | UUCUUUGUGAAAAGGUCAAGAUUAA | 2783 | TTAATCTTGACCTTTTCACAAAGAA | 3387 |
| 504 | AUUCAUCUGUUGGAUCUUGUAAACA | 2784 | TGTTTACAAGATCCAACAGATGAAT | 3388 |
| 505 | UUCAUCUGUUGGAUCUUGUAAACAU | 2785 | ATGTTTACAAGATCCAACAGATGAA | 3389 |
| 506 | UCAUCUGUUGGAUCUUGUAAACAUG | 2786 | CATGTTTACAAGATCCAACAGATGA | 3390 |
| 507 | CAUCUGUUGGAUCUUGUAAACAUGA | 2787 | TCATGTTTACAAGATCCAACAGATG | 3391 |
| 508 | AUCUGUUGGAUCUUGUAAACAUGAA | 2788 | TTCATGTTTACAAGATCCAACAGAT | 3392 |
| 509 | UCUGUUGGAUCUUGUAAACAUGAAA | 2789 | TTTCATGTTTACAAGATCCAACAGA | 3393 |
| 510 | CUGUUGGAUCUUGUAAACAUGAAAA | 2790 | TTTTCATGTTTACAAGATCCAACAG | 3394 |
| 511 | UGUUGGAUCUUGUAAACAUGAAAAG | 2791 | CTTTTCATGTTTACAAGATCCAACA | 3395 |
| 512 | GUUGGAUCUUGUAAACAUGAAAAGG | 2792 | CCTTTTCATGTTTACAAGATCCAAC | 3396 |
| 513 | UUGGAUCUUGUAAACAUGAAAAGGG | 2793 | CCCTTTTCATGTTTACAAGATCCAA | 3397 |
| 514 | UGGAUCUUGUAAACAUGAAAAGGGC | 2794 | GCCCTTTTCATGTTTACAAGATCCA | 3398 |
| 515 | GGAUCUUGUAAACAUGAAAAGGGCU | 2795 | AGCCCTTTTCATGTTTACAAGATCC | 3399 |
| 516 | GAUCUUGUAAACAUGAAAAGGGCUU | 2796 | AAGCCCTTTTCATGTTTACAAGATC | 3400 |
| 517 | AUCUUGUAAACAUGAAAAGGGCUUU | 2797 | AAAGCCCTTTTCATGTTTACAAGAT | 3401 |
| 518 | UCUUGUAAACAUGAAAAGGGCUUUA | 2798 | TAAAGCCCTTTTCATGTTTACAAGA | 3402 |
| 519 | CUUGUAAACAUGAAAAGGGCUUUAU | 2799 | ATAAAGCCCTTTTCATGTTTACAAG | 3403 |
| 520 | UUGUAAACAUGAAAAGGGCUUUAUU | 2800 | AATAAAGCCCTTTTCATGTTTACAA | 3404 |
| 521 | UGUAAACAUGAAAAGGGCUUUAUUU | 2801 | AAATAAAGCCCTTTTCATGTTTACA | 3405 |
| 522 | GUAAACAUGAAAAGGGCUUUAUUUU | 2802 | AAAATAAAGCCCTTTTCATGTTTAC | 3406 |
| 531 | AAAAGGGCUUUAUUUUCAAAAAUUA | 2803 | TAATTTTTGAAAATAAAGCCCTTTT | 3407 |
| 532 | AAAGGGCUUUAUUUUCAAAAAUUAA | 2804 | TTAATTTTTGAAAATAAAGCCCTTT | 3408 |
| 533 | AAGGGCUUUAUUUUCAAAAAUUAAC | 2805 | GTTAATTTTTGAAAATAAAGCCCTT | 3409 |
| 534 | AGGGCUUUAUUUUCAAAAAUUAACU | 2806 | AGTTAATTTTTGAAAATAAAGCCCT | 3410 |
| 535 | GGGCUUUAUUUUCAAAAAUUAACUU | 2807 | AAGTTAATTTTTGAAAATAAAGCCC | 3411 |
| 570 | GUAUAAAAUGCAACUGUUGAUUUCC | 2808 | GGAAATCAACAGTTGCATTTTATAC | 3412 |
| 571 | UAUAAAAUGCAACUGUUGAUUUCCU | 2809 | AGGAAATCAACAGTTGCATTTTATA | 3413 |
| 572 | AUAAAAUGCAACUGUUGAUUUCCUC | 2810 | GAGGAAATCAACAGTTGCATTTTAT | 3414 |
| 573 | UAAAAUGCAACUGUUGAUUUCCUCA | 2811 | TGAGGAAATCAACAGTTGCATTTTA | 3415 |
| 574 | AAAAUGCAACUGUUGAUUUCCUCAA | 2812 | TTGAGGAAATCAACAGTTGCATTTT | 3416 |
| 586 | UUGAUUUCCUCAACAUGGCUCACAA | 2813 | TTGTGAGCCATGTTGAGGAAATCAA | 3417 |
| 587 | UGAUUUCCUCAACAUGGCUCACAAA | 2814 | TTTGTGAGCCATGTTGAGGAAATCA | 3418 |
| 588 | GAUUUCCUCAACAUGGCUCACAAAU | 2815 | ATTTGTGAGCCATGTTGAGGAAATC | 3419 |
| 589 | AUUUCCUCAACAUGGCUCACAAAUU | 2816 | AATTTGTGAGCCATGTTGAGGAAAT | 3420 |
| 590 | UUUCCUCAACAUGGCUCACAAAUUU | 2817 | AAATTTGTGAGCCATGTTGAGGAAA | 3421 |
| 591 | UUCCUCAACAUGGCUCACAAAUUUC | 2818 | GAAATTTGTGAGCCATGTTGAGGAA | 3422 |
| 592 | UCCUCAACAUGGCUCACAAAUUUCU | 2819 | AGAAATTTGTGAGCCATGTTGAGGA | 3423 |
| 593 | CCUCAACAUGGCUCACAAAUUUCUA | 2820 | TAGAAATTTGTGAGCCATGTTGAGG | 3424 |
| 594 | CUCAACAUGGCUCACAAAUUUCUAU | 2821 | ATAGAAATTTGTGAGCCATGTTGAG | 3425 |
| 595 | UCAACAUGGCUCACAAAUUUCUAUC | 2822 | GATAGAAATTTGTGAGCCATGTTGA | 3426 |
| 596 | CAACAUGGCUCACAAAUUUCUAUCC | 2823 | GGATAGAAATTTGTGAGCCATGTTG | 3427 |
| 597 | AACAUGGCUCACAAAUUUCUAUCCC | 2824 | GGGATAGAAATTTGTGAGCCATGTT | 3428 |
| 598 | ACAUGGCUCACAAAUUUCUAUCCCA | 2825 | TGGGATAGAAATTTGTGAGCCATGT | 3429 |
| 599 | CAUGGCUCACAAAUUUCUAUCCCAA | 2826 | TTGGGATAGAAATTTGTGAGCCATG | 3430 |
| 600 | AUGGCUCACAAAUUUCUAUCCCAAA | 2827 | TTTGGGATAGAAATTTGTGAGCCAT | 3431 |
| 601 | UGGCUCACAAAUUUCUAUCCCAAAU | 2828 | ATTTGGGATAGAAATTTGTGAGCCA | 3432 |
| 602 | GGCUCACAAAUUUCUAUCCCAAAUC | 2829 | GATTTGGGATAGAAATTTGTGAGCC | 3433 |
| 603 | GCUCACAAAUUUCUAUCCCAAAUCU | 2830 | AGATTTGGGATAGAAATTTGTGAGC | 3434 |
| 604 | CUCACAAAUUUCUAUCCCAAAUCUU | 2831 | AAGATTTGGGATAGAAATTTGTGAG | 3435 |
| 605 | UCACAAAUUUCUAUCCCAAAUCUUU | 2832 | AAAGATTTGGGATAGAAATTTGTGA | 3426 |
| 606 | CACAAAUUUCUAUCCCAAAUCUUUU | 2833 | AAAAGATTTGGGATAGAAATTTGTG | 3437 |
| 607 | ACAAAUUUCUAUCCCAAAUCUUUUC | 2834 | GAAAAGATTTGGGATAGAAATTTGT | 3438 |
| 608 | CAAAUUUCUAUCCCAAAUCUUUUCU | 2835 | AGAAAAGATTTGGGATAGAAATTTG | 3439 |
| 609 | AAAUUUCUAUCCCAAAUCUUUUCUG | 2836 | CAGAAAAGATTTGGGATAGAAATTT | 3440 |
| 610 | AAUUUCUAUCCCAAAUCUUUUCUGA | 2837 | TCAGAAAAGATTTGGGATAGAAATT | 3441 |
| 611 | AUUUCUAUCCCAAAUCUUUUCUGAA | 2838 | TTCAGAAAAGATTTGGGATAGAAAT | 3442 |
| 612 | UUUCUAUCCCAAAUCUUUUCUGAAG | 2839 | CTTCAGAAAAGATTTGGGATAGAAA | 3443 |
| 613 | UUCUAUCCCAAAUCUUUUCUGAAGA | 2840 | TCTTCAGAAAAGATTTGGGATAGAA | 3444 |
| 644 | GUUUAGUUUUAAAACUGCACUGCCA | 2841 | TGGCAGTGCAGTTTTAAAACTAAAC | 3445 |
| 645 | UUUAGUUUUAAAACUGCACUGCCAA | 2842 | TTGGCAGTGCAGTTTTAAAACTAAA | 3446 |
| 646 | UUAGUUUUAAAACUGCACUGCCAAC | 2843 | GTTGGCAGTGCAGTTTTAAAACTAA | 3447 |
| 647 | UAGUUUUAAAACUGCACUGCCAACA | 2844 | TGTTGGCAGTGCAGTTTTAAAACTA | 3448 |
| 648 | AGUUUUAAAACUGCACUGCCAACAA | 2845 | TTGTTGGCAGTGCAGTTTTAAAACT | 3449 |
| 649 | GUUUUAAAACUGCACUGCCAACAAG | 2846 | CTTGTTGGCAGTGCAGTTTTAAAAC | 3450 |
| 650 | UUUUAAAACUGCACUGCCAACAAGU | 2847 | ACTTGTTGGCAGTGCAGTTTTAAAA | 3451 |
| 651 | UUUAAAACUGCACUGCCAACAAGUU | 2848 | AACTTGTTGGCAGTGCAGTTTTAAA | 3452 |
| 652 | UUAAAACUGCACUGCCAACAAGUUC | 2849 | GAACTTGTTGGCAGTGCAGTTTTAA | 3453 |
| 653 | UAAAACAGCACUGCCAACAAGUUCA | 2850 | TGAACTTGTTGGCAGTGCAGTTTTA | 3454 |
| 654 | AAAACUGCACUGCCAACAAGUUCAC | 2851 | GTGAACTTGTTGGCAGTGCAGTTTT | 3455 |
| 655 | AAACUGCACUGCCAACAAGUUCACU | 2852 | AGTGAACTTGTTGGCAGTGCAGTTT | 3456 |
| 656 | AAACUGCACUGCCAACAAGUUCACU | 2853 | AGTGAACTTGTTGGCAGTGCAGTTT | 3456 |
| 657 | ACUGCACUGCCAACAAGUUCACUUC | 2854 | GAAGTGAACTTGTTGGCAGTGCAGT | 3458 |
| 658 | CUGCACUGCCAACAAGUUCACUUCA | 2855 | TGAAGTGAACTTGTTGGCAGTGCAG | 3459 |
| 659 | UGCACUGCCAACAAGUUCACUUCAU | 2856 | ATGAAGTGAACTTGTTGGCAGTGCA | 3460 |
| 660 | GCACUGCCAACAAGUUCACUUCAUA | 2857 | TATGAAGTGAACTTGTTGGCAGTGC | 3461 |
| 661 | CACUGCCAACAAGUUCACUUCAUAU | 2858 | ATATGAAGTGAACTTGTTGGCAGTG | 3462 |
| 662 | ACUGCCAACAAGUUCACUUCAUAUA | 2859 | TATATGAAGTGAACTTGTTGGCAGT | 3463 |
| 663 | CUGCCAACAAGUUCACUUCAUAUAU | 2860 | ATATATGAAGTGAACTTGTTGGCAG | 3464 |
| 755 | UAAGUAUUUUUCAGGUCUUCACCAA | 2861 | TTGGTGAAGACCTGAAAAATACTTA | 3465 |
| 756 | AAGUAUUUUUCAGGUCUUCACCAAG | 2862 | CTTGGTGAAGACCTGAAAAATACTT | 3466 |
| 757 | AGUAUUUUUCAGGUCUUCACCAAGU | 2863 | ACTTGGTGAAGACCTGAAAAATACT | 3467 |
| 760 | AUUUUUCAGGUCUUCACCAAGUAUC | 2864 | GATACTTGGTGAAGACCTGAAAAAT | 3468 |
| 761 | UUUUUCAGGUCUUCACCAAGUAUCA | 2865 | TGATACTTGGTGAAGACCTGAAAAA | 3469 |
| 762 | UUUUCAGGUCUUCACCAAGUAUCAA | 2866 | TTGATACTTGGTGAAGACCTGAAAA | 3470 |
| 763 | UUUCAGGUCUUCACCAAGUAUCAAA | 2867 | TTTGATACTTGGTGAAGACCTGAAA | 3471 |
| 764 | UUCAGGUCUUCACCAAGUAUCAAAG | 2868 | CTTTGATACTTGGTGAAGACCTGAA | 3472 |
| 765 | UCAGGUCUUCACCAAGUAUCAAAGU | 2869 | ACTTTGATACTTGGTGAAGACCTGA | 3473 |
| 766 | CAGGUCUUCACCAAGUAUCAAAGUA | 2870 | TACTTTGATACTTGGTGAAGACCTG | 3474 |
| 813 | AUUCAAAAUAGUCCACUGACUCCUC | 2871 | GAGGAGTCAGTGGACTATTTTGAAT | 3475 |
| 814 | UUCAAAAUAGUCCACUGACUCCUCA | 2872 | TGAGGAGTCAGTGGACTATTTTGAA | 3476 |
| 815 | UCAAAAUAGUCCACUGACUCCUCAC | 2873 | GTGAGGAGTCAGTGGACTATTTTGA | 3477 |
| 816 | CAAAAUAGUCCACUGACUCCUCACA | 2874 | TGTGAGGAGTCAGTGGACTATTTTG | 3478 |
| 817 | AAAAUAGUCCACUGACUCCUCACAU | 2875 | ATGTGAGGAGTCAGTGGACTATTTT | 3479 |
| 818 | AAAUAGUCCACUGACUCCUCACAUC | 2876 | GATGTGAGGAGTCAGTGGACTATTT | 3480 |
| 819 | AAUAGUCCACUGACUCCUCACAUCU | 2877 | AGATGTGAGGAGTCAGTGGACTATT | 3481 |
| 820 | AUAGUCCACUGACUCCUCACAUCUG | 2878 | CAGATGTGAGGAGTCAGTGGACTAT | 3482 |
| 821 | UAGUCCACUGACUCCUCACAUCUGU | 2879 | ACAGATGTGAGGAGTCAGTGGACTA | 3483 |
| 822 | AGUCCACUGACUCCUCACAUCUGUU | 2880 | AACAGATGTGAGGAGTCAGTGGACT | 3484 |
| 823 | GUCCACUGACUCCUCACAUCUGUUA | 2881 | TAACAGATGTGAGGAGTCAGTGGAC | 3485 |
| 824 | UCCACUGACUCCUCACAUCUGUUAU | 2882 | ATAACAGATGTGAGGAGTCAGTGGA | 3486 |
| 825 | CCACUGACUCCUCACAUCUGUUAUC | 2883 | GATAACAGATGTGAGGAGTCAGTGG | 3487 |
| 911 | UUUUUCUAUGCCACAUUAACAUCUU | 2884 | AAGATGTGAAATGTGGCATAGAAAA | 3488 |
| 912 | UUUUCUAUGCCACAUUAACAUCUUU | 2885 | AAAGATGTTAATGTGGCATAGAAAA | 3489 |
| 913 | UUUCUAUGCCACAUUAACAUCUUUU | 2886 | AAAAGATGTTAATGTGGCATAGAAA | 3490 |
| 919 | UGCCACAUUAACAUCUUUUAAAGUU | 2887 | AACTTTAAAAGATGTTAATGTGGCA | 3491 |
| 920 | GCCACAUUAACAUCUUUUAAAGUUG | 2888 | CAACTTTAAAAGATGTTAATGTGGC | 3492 |
| 948 | AGAAUCAAGUAUGGAAAAGUAAGGC | 2889 | GCCTTACTTTTCCATACTTGATTCT | 3493 |
| 949 | GAAUCAAGUAUGGAAAAGUAAGGCC | 2890 | GGCCTTACTTTTCCATACTTGATTC | 3494 |
| 950 | AAUCAAGUAUGGAAAAGUAAGGCCA | 2891 | TGGCCTTACTTTTCCATACTTGATT | 3495 |
| 959 | UGGAAAAGUAAGGCCAUACUCUUAC | 2892 | GTAAGAGTATGGCCTTACTTTTCCA | 3496 |
| 960 | GGAAAAGUAAGGCCAUACUCUUACA | 2893 | TGTAAGAGTATGGCCTTACTTTTCC | 3497 |
| 1067 | CAUAUGAUCAACAGAUGAGAACUGG | 2894 | CCAGTTCTCATCTGTTGATCATATG | 3498 |
| 1069 | UAUGAUCAACAGAUGAGAACUGGUG | 2895 | CACCAGTTCTCATCTGTTGATCATA | 3499 |
| 1070 | AUGAUCAACAGAUGAGAACUGGUGG | 2896 | CCACCAGTTCTCATCTGTTGATCAT | 3500 |
| 1071 | UGAUCAACAGAUGAGAACUGGUGGU | 2897 | ACCACCAGTTCTCATCTGTTGATCA | 3501 |
| 1072 | GAUCAACAGAUGAGAACUGGUGGUU | 2898 | AACCACCAGTTCTCATCTGTTGATC | 3502 |
| 1073 | AUCAACAGAUGAGAACUGGUGGUUA | 2899 | TAACCACCAGTTCTCATCTGTTGAT | 3503 |
| 1074 | UCAACAGAUGAGAACUGGUGGUUAA | 2900 | TTAACCACCAGTTCTCATCTGTTGA | 3504 |
| 1075 | CAACAGAUGAGAACUGGUGGUUAAU | 2901 | ATTAACCACCAGTTCTCATCTGTTG | 3505 |
| 1078 | CAGAUGAGAACUGGUGGUUAAUAUG | 2902 | CATATTAACCACCAGTTCTCATCTG | 3506 |
| 1080 | GAUGAGAACUGGUGGUUAAUAUGUG | 2903 | CACATATTAACCACCAGTTCTCATC | 3507 |
| 1081 | AUGAGAACUGGUGGUUAAUAUGUGA | 2904 | TCACATATTAACCACCAGTTCTCAT | 3508 |
| 1082 | UGAGAACUGGUGGUUAAUAUGUGAC | 2905 | GTCACATATTAACCACCAGTTCTCA | 3509 |
| 1083 | GAGAACUGGUGGUUAAUAUGUGACA | 2906 | TGTCACATATTAACCACCAGTTCTC | 3510 |
| 1086 | AACUGGUGGUUAAUAUGUGACAGUG | 2907 | CACTGTCACATATTAACCACCAGTT | 3511 |
| 1087 | ACUGGUGGUUAAUAUGUGACAGUGA | 2908 | TCACTGTCACATATTAACCACCAGT | 3512 |
| 1088 | CUGGUGGUUAAUAUGUGACAGUGAG | 2909 | CTCACTGTCACATATTAACCACCAG | 3513 |
| 1089 | UGGUGGUUAAUAUGUGACAGUGAGA | 2910 | TCTCACTGTCACATATTAACCACCA | 3514 |
| 1141 | CAGAAUCUAAUCUUCAUUUAAGGCA | 2911 | TGCCTTAAATGAAGATTAGATTCTG | 3515 |
| 1150 | AUCUUCAUUUAAGGCACUGUAGUGA | 2912 | TCACTACAGTGCCTTAAATGAAGAT | 3516 |
| 1151 | UCUUCAUUUAAGGCACUGUAGUGAA | 2913 | TTCACTACAGTGCCTTAAATGAAGA | 3517 |
| 1153 | UUCAUUUAAGGCACUGUAGUGAAUU | 2914 | AATTCACTACAGTGCCTTAAATGAA | 3518 |
| 1161 | AGGCACUGUAGUGAAUUAUCUGAGC | 2915 | GCTCAGATAATTCACTACAGTGCCT | 3519 |
| 1162 | GGCACUGUAGUGAAUUAUCUGAGCU | 2916 | AGCTCAGATAATTCACTACAGTGCC | 3520 |
| 1211 | UAUCUUUGGAAUCAUGAAACCUUAA | 2917 | TTAAGGTTTCATGATTCCAAAGATA | 3521 |
| 1212 | AUCUUUGGAAUCAUGAAACCUUAAG | 2918 | CTTAAGGTTTCATGATTCCAAAGAT | 3522 |
| 1213 | UCUUUGGAAUCAUGAAACCUUAAGA | 2919 | TCTTAAGGTTTCATGATTCCAAAGA | 3523 |
| 1214 | CUUUGGAAUCAUGAAACCUUAAGAC | 2920 | GTCTTAAGGTTTCATGATTCCAAAG | 3524 |
| 1215 | UUUGGAAUCAUGAAACCUUAAGACU | 2921 | AGTCTTAAGGTTTCATGATTCCAAA | 3525 |
| 1216 | UUGGAAUCAUGAAACCUUAAGACUU | 2922 | AAGTCTTAAGGTTTCATGATTCCAA | 3526 |
| 1217 | UGGAAUCAUGAAACCUUAAGACUUC | 2923 | GAAGTCTTAAGGTTTCATGATTCCA | 3527 |
| 1218 | GGAAUCAUGAAACCUUAAGACUUCA | 2924 | TGAAGTCTTAAGGTTTCATGATTCC | 3528 |
| 1223 | CAUGAAACCUUAAGACUUCAGAAUG | 2925 | CATTCTGAAGTCTTAAGGTTTCATG | 3529 |
| 1230 | CCUUAAGACUUCAGAAUGAUUUUGC | 2926 | GCAAAATCATTCTGAAGTCTTAAGG | 3530 |
| 1231 | CUUAAGACUUCAGAAUGAUUUUGCA | 2927 | TGCAAAATCATTCTGAAGTCTTAAG | 3531 |
| 1232 | UUAAGACUUCAGAAUGAUUUUGCAG | 2928 | CTGCAAAATCATTCTGAAGTCTTAA | 3532 |
| 1233 | UAAGACUUCAGAAUGAUUUUGCAGG | 2929 | CCTGCAAAATCATTCTGAAGTCTTA | 3533 |
| 1234 | AAGACUUCAGAAUGAUUUUGCAGGU | 2930 | ACCTGCAAAATCATTCTGAAGTCTT | 3534 |
| 1235 | AGACUUCAGAAUGAUUUUGCAGGUU | 2931 | AACCTGCAAAATCATTCTGAAGTCT | 3535 |
| 1236 | GACUUCAGAAUGAUUUUGCAGGUUG | 2932 | CAACCTGCAAAATCATTCTGAAGTC | 3536 |
| 1237 | ACUUCAGAAUGAUUUUGCAGGUUGU | 2933 | ACAACCTGCAAAATCATTCTGAAGT | 3537 |
| 1238 | CUUCAGAAUGAUUUUGCAGGUUGUC | 2934 | GACAACCTGCAAAATCATTCTGAAG | 3538 |
| 1239 | UUCAGAAUGAUUUUGCAGGUUGUCU | 2935 | AGACAACCTGCAAAATCATTCTGAA | 3539 |
| 1240 | UCAGAAUGAUUUUGCAGGUUGUCUU | 2936 | AAGACAACCTGCAAAATCATTCTGA | 3540 |
| 1241 | CAGAAUGAUUUUGCAGGUUGUCUUC | 2937 | GAAGACAACCTGCAAAATCATTCTG | 3541 |
| 1242 | AGAAUGAUUUUGCAGGUUGUCUUCC | 2938 | GGAAGACAACCTGCAAAATCATTCT | 3542 |
| 1243 | GAAUGAUUUUGCAGGUUGUCUUCCA | 2939 | TGGAAGACAACCTGCAAAATCATTC | 3543 |
| 1244 | AAUGAUUUUGCAGGUUGUCUUCCAU | 2940 | ATGGAAGACAACCTGCAAAATCATT | 3544 |
| 1245 | AUGAUUUUGCAGGUUGUCUUCCAUU | 2941 | AATGGAAGACAACCTGCAAAATCAT | 3545 |
| 1246 | UGAUUUUGCAGGUUGUCUUCCAUUC | 2942 | GAATGGAAGACAACCTGCAAAATCA | 3546 |
| 1247 | GAUUUUGCAGGUUGUCUUCCAUUCC | 2943 | GGAATGGAAGACAACCTGCAAAATC | 3547 |
| 1248 | AUUUUGCAGGUUGUCUUCCAUUCCA | 2944 | TGGAATGGAAGACAACCTGCAAAAT | 3548 |
| 1249 | UUUUGCAGGUUGUCUUCCAUUCCAG | 2945 | CTGGAATGGAAGACAACCTGCAAAA | 3549 |
| 1250 | UUUGCAGGUUGUCUUCCAUUCCAGC | 2946 | GCTGGAATGGAAGACAACCTGCAAA | 3550 |
| 1251 | UUGCAGGUUGUCUUCCAUUCCAGCC | 2947 | GGCTGGAATGGAAGACAACCTGCAA | 3551 |
| 1252 | UGCAGGUUGUCUUCCAUUCCAGCCU | 2948 | AGGCTGGAATGGAAGACAACCTGCA | 3552 |
| 1253 | GCAGGUUGUCUUCCAUUCCAGCCUA | 2949 | TAGGCTGGAATGGAAGACAACCTGC | 3553 |
| 1254 | CAGGUUGUCUUCCAUUCCAGCCUAA | 2950 | TTAGGCTGGAATGGAAGACAACCTG | 3554 |
| 1255 | AGGUUGUCUUCCAUUCCAGCCUAAC | 2951 | GTTAGGCTGGAATGGAAGACAACCT | 3555 |
| 1256 | GGUUGUCUUCCAUUCCAGCCUAACA | 2952 | TGTTAGGCTGGAATGGAAGACAACC | 3556 |
| 1257 | GUUGUCUUCCAUUCCAGCCUAACAU | 2953 | ATGTTAGGCTGGAATGGAAGACAAC | 3557 |
| 1258 | UUGUCUUCCAUUCCAGCCUAACAUC | 2954 | GATGTTAGGCTGGAATGGAAGACAA | 3558 |
| 1259 | UGUCUUCCAUUCCAGCCUAACAUCC | 2955 | GGATGTTAGGCTGGAATGGAAGACA | 3559 |
| 1260 | GUCUUCCAUUCCAGCCUAACAUCCA | 2956 | TGGATGTTAGGCTGGAATGGAAGAC | 3560 |
| 1261 | UCUUCCAUUCCAGCCUAACAUCCAA | 2957 | TTGGATGTTAGGCTGGAATGGAAGA | 3561 |
| 1262 | CUUCCAUUCCAGCCUAACAUCCAAU | 2958 | ATTGGATGTTAGGCTGGAATGGAAG | 3562 |
| 1263 | UUCCAUUCCAGCCUAACAUCCAAUG | 2959 | CATTGGATGTTAGGCTGGAATGGAA | 3563 |
| 1264 | UCCAUUCCAGCCUAACAUCCAAUGC | 2960 | GCATTGGATGTTAGGCTGGAATGGA | 3564 |
| 1265 | CCAUUCCAGCCUAACAUCCAAUGCA | 2961 | TGCATTGGATGTTAGGCTGGAATGG | 3565 |
| 1266 | CAUUCCAGCCUAACAUCCAAUGCAG | 2962 | CTGCATTGGATGTTAGGCTGGAATG | 3566 |
| 1267 | AUUCCAGCCUAACAUCCAAUGCAGG | 2963 | CCTGCATTGGATGTTAGGCTGGAAT | 3567 |
| 1274 | CCUAACAUCCAAUGCAGGCAAGGAA | 2964 | TTCCTTGCCTGCATTGGATGTTAGG | 3568 |
| 1275 | CUAACAUCCAAUGCAGGCAAGGAAA | 2965 | TTTCCTTGCCTGCATTGGATGTTAG | 3569 |
| 1276 | UAACAUCCAAUGCAGGCAAGGAAAA | 2966 | TTTTCCTTGCCTGCATTGGATGTTA | 3570 |
| 1277 | AACAUCCAAUGCAGGCAAGGAAAAU | 2967 | ATTTTCCTTGCCTGCATTGGATGTT | 3571 |
| 1278 | ACAUCCAAUGCAGGCAAGGAAAAUA | 2968 | TATTTTCCTTGCCTGCATTGGATGT | 3572 |
| 1279 | CAUCCAAUGCAGGCAAGGAAAAUAA | 2969 | TTATTTTCCTTGCCTGCATTGGATG | 3573 |
| 1280 | AUCCAAUGCAGGCAAGGAAAAUAAA | 2970 | TTTATTTTCCTTGCCTGCATTGGAT | 3574 |
| 1281 | UCCAAUGCAGGCAAGGAAAAUAAAA | 2971 | TTTTATTTTCCTTGCCTGCATTGGA | 3575 |
| 1282 | CCAAUGCAGGCAAGGAAAAUAAAAG | 2972 | CTTTTATTTTCCTTGCCTGCATTGG | 3576 |
| 1283 | CAAUGCAGGCAAGGAAAAUAAAAGA | 2973 | TCTTTTATTTTCCTTGCCTGCATTG | 3577 |
| 1284 | AAUGCAGGCAAGGAAAAUAAAAGAU | 2974 | ATCTTTTATTTTCCTTGCCTGCATT | 3578 |
| 1285 | AUGCAGGCAAGGAAAAUAAAAGAUU | 2975 | AATCTTTTATTTTCCTTGCCTGCAT | 3579 |
| 1286 | UGCAGGCAAGGAAAAUAAAAGAUUU | 2976 | AAATCTTTTATTTTCCTTGCCTGCA | 3580 |
| 1287 | GCAGGCAAGGAAAAUAAAAGAUUUC | 2977 | GAAATCTTTTATTTTCCTTGCCTGC | 3581 |
| 1301 | UAAAAGAUUUCCAGUGACAGAAAAA | 2978 | TTTTTCTGTCACTGGAAATCTTTTA | 3582 |
| 1302 | AAAAGAUUUCCAGUGACAGAAAAAU | 2979 | ATTTTTCTGTCACTGGAAATCTTTT | 3583 |
| 1393 | UAUAUUUUGAAAUGAACUUGUUGGC | 2980 | GCCAACAAGTTCATTTCAAAATATA | 3584 |
| 1394 | AUAUUUUGAAAUGAACUUGUUGGCC | 2981 | GGCCAACAAGTTCATTTCAAAATAT | 3585 |
| 1395 | UAUUUUGAAAUGAACUUGUUGGCCC | 2982 | GGGCCAACAAGTTCATTTCAAAATA | 3586 |
| 1396 | AUUUUGAAAUGAACUUGUUGGCCCA | 2983 | TGGGCCAACAAGTTCATTTCAAAAT | 3587 |
| 1397 | UUUUGAAAUGAACUUGUUGGCCCAU | 2984 | ATGGGCCAACAAGTTCATTTCAAAA | 3588 |
| 1398 | UUUGAAAUGAACUUGUUGGCCCAUC | 2985 | GATGGGCCAACAAGTTCATTTCAAA | 3589 |
| 1399 | UUGAAAUGAACUUGUUGGCCCAUCU | 2986 | AGATGGGCCAACAAGTTCATTTCAA | 3590 |
| 1400 | UGAAAUGAACUUGUUGGCCCAUCUA | 2987 | TAGATGGGCCAACAAGTTCATTTCA | 3591 |
| 1401 | GAAAUGAACUUGUUGGCCCAUCUAU | 2988 | ATAGATGGGCCAACAAGTTCATTTC | 3592 |
| 1402 | AAAUGAACUUGUUGGCCCAUCUAUU | 2989 | AATAGATGGGCCAACAAGTTCATTT | 3593 |
| 1403 | AAUGAACUUGUUGGCCCAUCUAUUA | 2990 | TATTAGATGGGCCAACAAGTTCATT | 3594 |
| 1404 | AUGAACUUGUUGGCCCAUCUAUUAC | 2991 | GTATTAGATGGGCCAACAAGTTCAT | 3595 |
| 1405 | UGAACUUGUUGGCCCAUCUAUUACA | 2992 | TGTAATAGATGGGCCAACAAGTTCA | 3596 |
| 1406 | GAACUUGUUGGCCCAUCUAUUACAU | 2993 | ATGTAATAGATGGGCCAACAAGTTC | 3597 |
| 1407 | AACUUGUUGGCCCAUCUAUUACAUC | 2994 | GATGTAATAGATGGGCCAACAAGTT | 3598 |
| 1408 | ACUUGUUGGCCCAUCUAUUACAUCU | 2995 | AGATGTAATAGATGGGCCAACAAGT | 3599 |
| 1409 | CUUGUUGGCCCAUCUAUUACAUCUA | 2996 | TAGATGTAATAGATGGGCCAACAAG | 3600 |
| 1410 | UUGUUGGCCCAUCUAUUACAUCUAC | 2997 | GTAGATGTAATAGATGGGCCAACAA | 3601 |
| 1411 | UGUUGGCCCAUCUAUUACAUCUACA | 2998 | TGTAGATGTAATAGATGGGCCAACA | 3602 |
| 1412 | GUUGGCCCAUCUAUUACAUCUACAG | 2999 | CTGTAGATGTAATAGATGGGCCAAC | 3603 |
| 1413 | UUGGCCCAUCUAUUACAUCUACAGC | 3000 | GCTGTAGATGTAATAGATGGGCCAA | 3604 |
| 1414 | UGGCCCAUCUAUUACAUCUACAGCU | 3001 | AGCTGTAGATGTAATAGATGGGCCA | 3605 |
| 1415 | GGCCCAUCUAUUACAUCUACAGCUG | 3002 | CAGCTGTAGATGTAATAGATGGGCC | 3606 |
| 1416 | GCCCAUCUAUUACAUCUACAGCUGA | 3003 | TCAGCTGTAGATGTAATAGATGGGC | 3607 |
| 1422 | CUAUUACAUCUACAGCUGACCCUUG | 3004 | CAAGGGTCAGCTGTAGATGTAATAG | 3608 |
| 1423 | UAUUACAUCUACAGCUGACCCUUGA | 3005 | TCAAGGGTCAGCTGTAGATGTAATA | 3609 |
| 1424 | AUUACAUCUACAGCUGACCCUUGAA | 3006 | TTCAAGGGTCAGCTGTAGATGTAAT | 3610 |
| 1425 | UUACAUCUACAGCUGACCCUUGAAC | 3007 | GTTCAAGGGTCAGCTGTAGATGTAA | 3611 |
| 1426 | UACAUCUACAGCUGACCCUUGAACA | 3008 | TGTTCAAGGGTCAGCTGTAGATGTA | 3612 |
| 1427 | ACAUCUACAGCUGACCCUUGAACAU | 3009 | ATGTTCAAGGGTCAGCTGTAGATGT | 3613 |
| 1428 | AUCUACAGCUGACCCUUGAACAUGG | 3010 | CATGTTCAAGGGTCAGCTGTAGATG | 3614 |
| 1429 | AUCUACAGCUGACCCUUGAACAUGG | 3011 | CCATGTTCAAGGGTCAGCTGTAGAT | 3615 |
| 1442 | CCUUGAACAUGGGGGUUAGGGGAGC | 3012 | GCTCCCCTAACCCCCATGTTCAAGG | 3616 |
| 1443 | CUUGAACAUGGGGGUUAGGGGAGCU | 3013 | AGCTCCCCTAACCCCCATGTTCAAG | 3617 |
| 1444 | UUGAACAUGGGGGUUAGGGGAGCUG | 3014 | CAGCTCCCCTAACCCCCATGTTCAA | 3618 |
| 1445 | UGAACAUGGGGGUUAGGGGAGCUGA | 3015 | TCAGCTCCCCTAACCCCCATGTTCA | 3619 |
| 1446 | GAACAUGGGGGUUAGGGGAGCUGAC | 3016 | GTCAGCTCCCCTAACCCCCATGTTC | 3620 |
| 1447 | AACAUGGGGGUUAGGGGAGCUGACA | 3017 | TGTCAGCTCCCCTAACCCCCATGTT | 3621 |
| 1448 | ACAUGGGGGUUAGGGGAGCUGACAA | 3018 | TTGTCAGCTCCCCTAACCCCCATGT | 3622 |
| 1449 | CAUGGGGGUUAGGGGAGCUGACAAU | 3019 | ATTGTCAGCTCCCCTAACCCCCATG | 3623 |
| 1450 | AUGGGGGUUAGGGGAGCUGACAAUU | 3020 | AATTGTCAGCTCCCTAACCCCCCAT | 3624 |
| 1451 | UGGGGGUUAGGGGAGCUGACAAUUC | 3021 | GAATTGTCAGCTCCCTAACCCCCCA | 3625 |
| 1452 | GGGGGUUAGGGGAGCUGACAAUUCG | 3022 | CGAATTGTCAGCTCCCCTAACCCCC | 3626 |
| 1453 | GGGGUUAGGGGAGCUGACAAUUCGU | 3023 | ACGAATTGTCAGCTCCCCTAACCCC | 3627 |
| 1454 | GGGUUAGGGGAGCUGACAAUUCGUG | 3024 | CACGAATTGTCAGCTCCCCTAACCC | 3628 |
| 1455 | GGUUAGGGGAGCUGACAAUUCGUGG | 3025 | CCACGAATTGTCAGCTCCCCTAACC | 3629 |
| 1456 | GUUAGGGGAGCUGACAAUUCGUGGG | 3026 | CCCACGAATTGTCAGCTCCCCTAAC | 3630 |
| 1457 | UUAGGGAGCUGACAAUUCGUGGGGU | 3027 | ACCCACGAATTGTCAGTCCCCTAAA | 3631 |
| 1458 | UAGGGGAGCUGACAAUUCGUGGGUC | 3028 | GACCCACGAATTGTCAGCTCCCCTA | 3632 |
| 1459 | AGGGGAGCUGACAAUUCGUGGGUCC | 3029 | GGACCCACGAATTGTCAGCTCCCCT | 3633 |
| 1460 | GGGGAGCUGACAAUUCGUGGGUCCG | 3030 | CGGACCCACGAATTGTCAGCTCCCC | 3634 |
| 1462 | GGAGCUGACAAUUCGUGGGUCCGCA | 3031 | TGCGGACCCACGAATTGTCAGCTCC | 3635 |
| 1463 | GAGCUGACAAUUCGUGGGUCCGCAA | 3032 | TTGCGGACCCACGAATTGTCAGCTC | 3636 |
| 1464 | AGCUGACAAUUCGUGGGUCCGCAAA | 3033 | TTTGCGGACCCACGAATTGTCAGCT | 3637 |
| 1465 | GCUGACAAUUCGUGGGUCCGCAAAA | 3034 | TTTTGCGGACCCACGAATTGTCAGC | 3638 |
| 1466 | CUGACAAUUCGUGGGUCCCGCAAAU | 3035 | ATTTTGCGGACCCACGAATTGTCAG | 3639 |
| 1467 | UGACAAUUCGUGGGUCCGCAAAAUC | 3036 | GATTTTGCGGACCCACGAATTGTCA | 3640 |
| 1468 | GACAAUUCGUGGGUCCGCAAAAUCU | 3037 | AGATTTTGCGGACCCACGAATTGTC | 3641 |
| 1469 | ACAAUUCGUGGGUCCGCAAAAUCUU | 3038 | AAGATTTTGCGGACCCACGAATTGT | 3642 |
| 1470 | CAAUUCGUGGGUCCGCAAAAUCUUA | 3039 | TAAGATTTTGCGGACCCACGAATTG | 3643 |
| 1471 | AAUUCGUGGGUCCGCAAAAUCUUAA | 3040 | TTAAGATTTTGCGGACCCACGAATT | 3644 |
| 1472 | AUUCGUGGGUCCGCAAAAUCUUAAC | 3041 | GTTAAGATTTTGCGGACCCACGAAT | 3645 |
| 1473 | UUCGUGGGUCCGCAAAAUCUUAACU | 3042 | AGTTAAGATTTTGCGGACCCACGAA | 3646 |
| 1474 | UCGUGGGUCCGCAAAAUCUUAACUA | 3043 | TAGTTAAGATTTTGCGGACCCACGA | 3647 |
| 1475 | CGUGGGUCCGCAAAAUCUUAACUAC | 3044 | GTAGTTAAGATTTTGCGGACCACCG | 3648 |
| 1476 | GUGGGUCCGCAAAAUCUUAACUACC | 3045 | GGTAGTTAAGATTTTGCGGACCCAC | 3649 |
| 1477 | UGGGUCCGCAAAAUCUUAACUACCU | 3046 | AGGTAGTTAAGATTTTGCGGACCCA | 3650 |
| 1478 | GGGUCCGCAAAAUCUUAACUACCUA | 3047 | TAGGTAGTTAAGATTTTGCGGACCC | 3651 |
| 1479 | GGUCCGCAAAAUCUUAACUACCUAA | 3048 | TTAGGTAGTTAAGATTTGCGGAACC | 3652 |
| 1480 | GUCCGCAAAAUCUUAACUACCUAAU | 3049 | ATTAGGTAGTTAAGATTTTGCGGAC | 3653 |
| 1481 | UCCGCAAAAUCUUAACUACCUAAUA | 3050 | TATTAGGTAGTTAAGATTTTGCGGA | 3654 |

Input Sequence = PLN
Oligo Length = 25
PLN (Homo sapiens phospholamban (PLN) mRNA; 1635 bp)

What we claim is:

1. A nucleic acid molecule which down regulates expression of a phospholamban gene.

2. The nucleic acid of claim 1, wherein said nucleic acid molecule is used to treat heart disease.

3. The nucleic acid molecule of claim 1, wherein said nucleic acid molecule is an enzymatic nucleic acid molecule.

4. The nucleic acid molecule of claim 3, wherein said enzymatic nucleic acid molecule has an endonuclease activity to cleave RNA encoded by said phospholamban gene

5. The nucleic acid of claim 4, wherein a binding arm of said enzymatic nucleic acid molecule comprise sequences complementary to any of sequences defined as sequence ID Nos. 1-1136.

6. The nucleic acid molecule of claim 4, wherein said enzymatic nucleic acid molecule comprises any of sequences defined as sequence ID Nos. 1137-2675.

7. The nucleic acid molecule of claim 1, wherein said nucleic acid molecule is an antisense nucleic acid molecule.

8. A nucleic acid molecule of claim 7, wherein said antisense nucleic acid molecule comprises sequence complementary to any of sequence defined as sequence ID Nos. 1-1136, and 2447-3050.

9. A nucleic acid molecule of claim 7, wherein said antisense molecule comprises any of sequences defined as sequence ID Nos. 3051-3654.

10. The nucleic acid molecule of claim 4, wherein said enzymatic nucleic acid molecule is in a hammerhead (HH) motif.

11. The nucleic acid molecule of claim 4, wherein said enzymatic nucleic acid molecule is in a hairpin, hepatitis Delta virus, group I intron, VS nucleic acid, amberzyme, zinzyme or RNAse P nucleic acid motif.

12. The nucleic acid molecule of claim 11, wherein said zinzyme motif comprises sequences complementary to any of substrate sequences shown in Table VI.

13. The nucleic acid molecule of claim 11, wherein said amberzyme motif comprises sequences complementary to any of substrate sequences shown in Table VIII.

14. The nucleic acid molecule of claim 4, wherein said enzymatic nucleic acid molecule is in a NCH motif.

15. The nucleic acid molecule of claim 4, wherein said enzymatic nucleic acid molecule is in a G-cleaver motif.

16. The nucleic acid molecule of claim 4, wherein said enzymatic nucleic acid molecule is a DNAzyme.

17. The nucleic acid molecule of claim 4, wherein said enzymatic nucleic acid molecule comprises between 12 and 100 bases complementary to the RNA.

18. The nucleic acid of claim 4, wherein said enzymatic nucleic acid molecule comprises between 14 and 24 bases complementary to the RNA of said region.

19. The nucleic acid molecule of claim 1, wherein said nucleic acid is chemically synthesized.

20. The nucleic acid molecule of claim 1, wherein said nucleic acid comprises at least one 2′-sugar modification.

21. The nucleic acid molecule of claim 1, wherein said nucleic acid comprises at least one nucleic acid base modification.

22. The nucleic acid molecule of claim 1, wherein said nucleic acid comprises at least one phosphate backbone modification.

23. A mammalian cell including the nucleic acid molecule of claim 1, wherein said mammalian cell is not a living human.

24. The mammalian cell of claim 23, wherein said mammalian cell is a human cell.

25. A method of inhibiting phospholamban activity in a cell, comprising the step of contacting said cell with the nucleic acid molecule of claim 1, under conditions suitable for said inhibition.

26. A method of treatment of a patient having a condition associated with the level of phospholamban, comprising contacting cells of said patient with the nucleic acid molecule of claim 1, under conditions suitable for said treatment.

27. The method of claim 26 further comprising the use of one or more drug therapies under conditions suitable for said treatment.

28. A method of cleaving RNA encoded by a phospholamban gene, comprising, contacting the enzymatic nucleic acid molecule of claim 4, with said RNA under conditions suitable for the cleavage of said RNA.

29. The method of claim 28, wherein said cleavage is carried out in the presence of a divalent cation.

30. The method of claim 29, wherein said divalent cation is Mg²⁺.

31. The nucleic acid molecule of claim 1, wherein said nucleic acid comprises a cap structure, wherein the cap structure is at the 5′-end or 3′-end or both the 5′-end and the 3′-end.

32. The nucleic acid molecule of claim 10, wherein said hammerhead motif comprises sequences complementary to any of sequences shown as Seq ID Nos 1-433.

33. The enzymatic nucleic acid molecule of claim 14, wherein said NCH motif comprises sequences complementary to any of sequences shown as Seq ID Nos 434-731.

34. The enzymatic nucleic acid molecule of claim 15, wherein said G-cleaver motif comprises sequences complementary to any of sequences shown as Seq ID Nos 732-814.

35. The enzymatic nucleic acid molecule of claim 16, wherein said DNAzyme comprises sequences complementary to any of the substrate sequences shown in Table VII.

36. The method of any of claims 25 or 27, wherein said nucleic acid molecule is an enzymatic nucleic acid molecule.

37. The method of any of claims 25 or 27, wherein said nucleic acid molecule is an antisense nucleic acid molecule.

38. The method of claim 36, wherein said enzymatic nucleic acid molecule is selected from the group consisting of hammerhead motif, hairpin motif, NCH motif, G-cleaver motif, zinzyme motif, amberzyme motif and DNAzyme.

39. The method of claim 28, wherein said enzymatic nucleic acid molecule is selected from the group consisting of hammerhead motif, hairpin motif, NCH motif, G-cleaver motif, zinzyme motif, amberzyme motif and DNAzyme.

40. An expression vector comprising nucleic acid sequence encoding at least one nucleic acid molecule of claim 1, in a manner which allows expression of that nucleic acid molecule.

41. A mammalian cell including an expression vector of claim 40, wherein said mammalian cell is not a living human.

42. The mammalian cell of claim 41, wherein said mammalian cell is a human cell.

43. The expression vector of claim 40, wherein said nucleic acid molecule is an enzymatic nucleic acid molecule.

44. The expression vector of claim 40, wherein said nucleic acid molecule is an antisense nucleic acid molecule.

45. The expression vector of claim 40, wherein said expression vector comprises sequence encoding at least two said nucleic acid molecules, which may be same or different.

46. The expression vector of claim 45, wherein one said nucleic acid molecule is an antisense nucleic acid molecule and the second said nucleic acid molecule is an enzymatic nucleic acid molecule.

47. A method for treatment of heart disease comprising the step of administering to a patient the nucleic acid molecule of claim 1 under conditions suitable for said treatment.

48. The method of claim 47, wherein said heart disease is heart failure.

49. The method of claim 47, wherein said heart disease is congestive heart failure.

50. The method of claim 47, wherein said nucleic acid molecule is an antisense nucleic acid molecule.

51. The method of claim 47, wherein said nucleic acid molecule is an enzymatic nucleic acid molecule.

52. A method for treatment of pressure overload hypertrophy, or dilated cardiomyopathy, or both, comprising the step of administering to a patient the nucleic acid molecule of claim 1 under conditions suitable for said treatment.

53. The method of claim 52, wherein said nucleic acid molecule is an antisense nucleic acid molecule.

54. The method of claim 52, wherein said nucleic acid molecule is an enzymatic nucleic acid molecule.

55. The method of claim 47, wherein said method further comprises administering to said patient the nucleic acid molecule in conjunction with one or more of other therapies.

56. The method of claim 52, wherein said method further comprises administering to said patient the nucleic acid molecule in conjunction with one or more of other therapies.

57. The nucleic acid molecule of any of claim 4, wherein said enzymatic nucleic acid molecule comprises at least five ribose residues; at least ten 2′-O-methyl modifications, and a 3′-end modification.

58. The nucleic acid molecule of claim 57, wherein said enzymatic nucleic acid molecule further comprises phosphorothioate linkages on at least three of the 5′ terminal nucleotides.

59. The nucleic acid molecule of claim 57, wherein said 3′-end modification is 3′-3′ inverted abasic moiety.

60. The nucleic acid molecule of claim 16, wherein said DNAzyme comprises at least ten 2′-O-methyl modifications and a 3′-end modification.

61. The nucleic acid molecule of claim 60, wherein said DNAzyme further comprises phosphorothioate linkages on at least three of the 5′ terminal nucleotides.

62. The nucleic acid molecule of claim 60, wherein said 3′-end modification is 3′-3′ inverted abasic moiety.
